(19) United States
(12) Patent Application Publication
(10) Pub. No.: US 2026/0263538 A1
(43) Pub. Date: Sep. 10, 2026

(54) USES OF BIFIDOBACTERIUM LONGUM TRANSITIONAL MICROORGANISM

(71) Applicant: SOCIÉTÉ DES PRODUITS NESTLÉ S.A., Vevey (CH)

(72) Inventors: Claire Laurence Lucie Marie BOULANGE, Oron-la-Ville (CH); Cheong Kwet Choy KWONG CHUNG, Lausanne (CH); Carine BLANCHARD, Le Mont-sur-Lausanne (CH); Sébastien HOLVOET, Montpreveyres (CH); Jean-Baptiste CAVIN, Forel (CH); Simona RAMBOUSEK, Saint-Sulpice (CH)

(21) Appl. No.: 18/881,563

(22) PCT Filed: Jul. 6, 2023
(86) PCT No.: PCT/EP2023/068675

§ 371 Date: Jan. 6, 2025

(30) Foreign Application Priority Data
Jul. 8, 2022 (EP) 22183945.9
Nov. 1, 2022 (EP) 22204954.6

(51) Int. Cl. A61K 35/745; A61K 31/702; A61K 39/00; A61K 39/35; A61P 37/08

(52) U.S. Cl. CPC A61K 35/745; A61K 31/702; A61K 39/35; A61P 37/08; A61K 2039/52; A61K 2039/58

(57) ABSTRACT

Treating and/or preventing an allergy and/or allergic sensitization The present invention relates to a Bifidobacterium longum transitional microorganism for use in treating and/or preventing an allergy and/or allergic sensitization in an infant or young child.

### FIELD OF THE INVENTION

The present invention is related to probiotics and prebiotics, in particular a Bifidobacterium longum transitional microorganism or a prebiotic that promotes the growth and/or survival of a Bifidobacterium longum transitional microorganism for use in treating and/or preventing an allergy and/or allergic sensitization in an infant or young child.

### BACKGROUND OF THE INVENTION

Allergies are among the most common health problems affecting patients of all ages. The prevalence of allergies has increased rapidly over the past decades. It has been estimated that over a third of the worldwide population is affected, to the extent that allergy has been considered as the new epidemic of the industrialized countries. The reasons for the steady increase in allergic diseases are not yet fully understood. Genetic background of the host is a prominent factor, and recently discovered genes have been shown to be associated with respiratory allergies/asthma and skin symptoms. Environmental factors such as lifestyle, pollution, decreasing family size, and reduction of microbial stimulation of the immune system in early life stage as a consequence of an improved hygienic situation seem to play an important role in the high prevalence and higher severity of allergic manifestations.

Allergic sensitization in childhood, especially in early childhood and especially to food allergens, is critical and the development of an “allergic phenotype” or “atopy” has been shown to facilitate subsequent sensitization to other allergens. Hence allergies in childhood can be the first step of an allergic cascade leading to multiple allergies later in life, a process commonly referred to as the “Atopic March”. For example, children with persistent food hypersensitivity early in life have a dramatically increased risk to develop allergic rhinitis (hay fever) or asthma later in childhood. Children with milder forms of food hypersensitivity also have increased risk for development of respiratory allergies but to a lesser degree than children with persistent food hypersensitivity. Therefore, attenuating the severity of food hypersensitivity may be crucial for slowing down the “Atopic March”. In this context the management of allergic episodes and the prevention of allergies are, in childhood and infancy, of the highest importance.

The immune system of infants is actively developing throughout the few first years of life. Acting on, preventing, avoiding, managing, reducing or modulating the allergic reactions at an early age can influence the allergic profile not only in the short term but also longer term for later in life.

Food allergens are among the first allergens that infants encounter in their early life: typically, cow's milk proteins may be encountered by infants not receiving exclusive breast feeding. Milk-proteins are indeed among the most frequently observed causes for food allergy in infancy, followed by eggs and wheat proteins. In general, food allergies can manifest in cutaneous (rash, eczema, others) and gastrointestinal symptoms (abdominal cramps; pain, especially in the abdomen; vomiting) in infants and young children. Food allergies are the most common trigger of severe allergic reactions, which may lead to life-threatening anaphylaxis. Further sensitization and episodes of allergies can also appear when the infant/young child is exposed to a novel food such as cereals, vegetables, fruits, nuts or fish, and also to air-borne allergens such as pollen, house dust mites and animal dander. Adults are affected to a large extent by contact and respiratory allergies. Data from the WHO [Clark, M. J. and. Million, R. P (2009) Allergic rhinitis: market evolution, Nature Reviews, Drug Discovery, 8, p. 271-272] indicates that up to 30-40% of the world's population suffer from some form of respiratory allergy.

Animals, particularly small animals such as pets- and especially companion animals such as dogs and cats, may also suffer from food allergies and food intolerances, as well as environmental allergens. These typically manifest in similar symptoms to humans, e.g. gastrointestinal disturbances such as diarrhoea, vomiting and abdominal discomfort, and also dermatitis or pruritis. In small animals, particularly dogs, the most frequent cause of chronic diarrhoea is food-responsive enteropathy (diet-responsive enteropathy or food-responsive diarrhoea).

There remains a need to develop new strategies for treating and/or preventing an allergy and/or allergic sensitization.

### SUMMARY OF THE INVENTION

The present inventors have determined that a Bifidobacterium longum subspecies microorganism (B. longum transitional) of a clade that is present in the gut microbiome of the transitional feeding period of mammals, particularly humans, may have beneficial effects on reducing the risk of developing an allergy and/or allergic sensitization. For example, the inventors have shown that the B. longum transitional microorganisms may be capable of modulating gut barrier permeability and/or promoting an anti-inflammatory and/or tolerogenic environment in the gut microbiota during the weaning period.

Thus, in a first aspect the present invention provides a Bifidobacterium longum transitional microorganism for use in treating and/or preventing an allergy and/or allergic sensitization in an infant or young child.

The invention further provides a prebiotic for use in treating and/or preventing an allergy and/or allergic sensitization in an infant or young child by promoting the growth and/or survival of a Bifidobacterium longum transitional microorganism in the gut of the infant or young child, wherein the prebiotic is: i. a glycan substrate, suitably selected from the group recited in any of Tables 1 to 3; and/or ii. a human milk oligosaccharide (HMO), suitably selected from the group consisting of 2′-O-fucosyllactose (2′-FL), 3′-O-fucosyllactose (3′-FL), lactodifucotetraose/difucosyllactose (di-FL), 3′-O-sialyllactose (3′-SL), 6′-O-sialyllactose (6′-SL), lacto-N-tetraose (LNT) and lacto-N-neotetraose (LNnT) and any combination thereof.

The invention also provides a combination of a Bifidobacterium longum transitional microorganism and a prebiotic for use in treating and/or preventing an allergy and/or allergic sensitization in an infant or young child; wherein the prebiotic is: i. a glycan substrate, suitably selected from the group recited in any of Tables 1 to 3; and/or ii. a human milk oligosaccharide (HMO), suitably selected from the group consisting of 2′-O-fucosyllactose (2′-FL), 3-O-fucosyllactose (3-FL), lactodifucotetraose/difucosyllactose (di-FL), 3′-O-sialyllactose (3′-SL), 6′-O-sialyllactose (6′-SL), lacto-N-tetraose (LNT) and lacto-N-neotetraose (LNnT) and any combination thereof.

The invention further provides a prebiotic for use in treating and/or preventing an allergy and/or allergic sensitization in an infant or young child by promoting the growth of a Bifidobacterium longum transitional microorganism in the gut of the infant or young child.

The invention also provides a combination of a Bifidobacterium longum transitional microorganism and a prebiotic for use in treating and/or preventing an allergy and/or allergic sensitization in an infant or young child.

In a further aspect, the invention relates to the use of a Bifidobacterium longum transitional microorganism, prebiotic or combination as defined herein for promoting immune tolerance in an infant or young child, preferably by promoting the growth and/or survival of a Bifidobacterium longum transitional microorganism in the gut of the infant or young child.

In another aspect, the invention relates to the use of a Bifidobacterium longum transitional microorganism, prebiotic or combination as defined herein for modulating gut barrier permeability in an infant or young child, preferably by promoting the growth of a Bifidobacterium longum transitional microorganism in the gut of the infant or young child.

### BRIEF DESCRIPTION OF THE DRAWINGS

FIG. 1—Average Nucleotide Identity (ANI) UPGMA based phylogenetic tree of strains belonging to the B. longum species. The scale represents the percentage of identity at each branch point.

FIG. 2—Transepithelial electrical resistance (TEER) of Caco-2 monolayers after apical treatment with 2×10⁶ CFU of probiotic strains. TEER was measured 2 h, 4 h, 6 h and 24 h after treatment, each value was normalized to its corresponding Oh value and is shown as percentage of initial value. Data are plotted as mean±SEM. For each concentration, differences between the complete medium (CM) control and treatments per timepoint were assessed using a two-way ANOVA with Dunnett's multiple comparisons test and statistical differences are represented by (*). (*)=p<0.05; (**)=p<0.01; (***)=p<0.001 and (****)=p<0.0001. NCC5000-5004: B. longum transitional strains, NCC2818: B. animalis subsp. lactis and NCC3001: B. longum subsp. longum

FIG. 3—Peripheral blood mononuclear cells (PBMC) were stimulated for 36 hours in the presence of different bacterial strains including all transitional B. longum isolates at 10⁷ CFU/ml and probiotic strains. Cell culture supernatants were collected to assess cytokine expression for IL-10 and IL-12p40 by ELISA. Standard curve for each cytokine was used to calculate absolute amount (picogram/ml) from optical density readouts. NCC5000-5004: B. longum transitional strains, NCC2818: B. animalis subsp. lactis, NCC3089: B. longum subsp. infantis, NCC4007: L. rhamnosus and NCC2705: B. longum subsp. longum

FIG. 4—Representation of glycoside hydrolases (GH) and polysaccharide lyases (PL) in the genomes of the B. longum clade. Heatmap shows presence (light) and absence (dark) of GH and PL genes, and the size of the circles represent the number of these genes per genome of a particular strain.

FIG. 5—Growth of B. longum transitional strain NCC 5001 was promoted in a complex gut microbiota community by pectin (sugar beet) and arabinogalactan (larch wood). P****<0.0001, ***<0.001, **<0.01, *<0.05, one-way ANOVA with uncorrected Fisher's LSD.

FIG. 6—Growth of B. longum transitional strain NCC 5002 was promoted in a complex gut microbiota community by arabinogalactan (larch wood) and starch (potato). P****<0.0001, ***<0.001, **<0.01, *<0.05, one-way ANOVA with uncorrected Fisher's LSD.

FIG. 7—Representative CAZyme sequences

FIG. 8—Schematic representation of the organization of the genes implicated in the degradation and the metabolization of fucosylated human milk oligosaccharides in the B. longum transitional strains, compared to B. longum subsp. infantis ATCC 15697 and B. kashiwanohense DSM 21854. Values represent percentage (%) of identity between the different genes.

FIG. 9—Growth of B. longum transitional strains and B. longum subsp. infantis LMG 11588 on glucose, 2′-FL or 3-FL as sole carbon source (0.5% final). Significant differences between 2′-FL and 3-FL growth for each strain were calculated using one-way ANOVA, followed by a Sidak's multiple comparison test (ns=non-significant, *p-value <0.05, **p-value <0.01).

FIG. 10—Growth ratios of 3-FL over 2′-FL of B. longum transitional strains and B. longum subsp. infantis LMG 11588.

FIG. 11—Riboflavin biosynthesis genes present in B. longum transitional strains. Panel A shows the organization of the riboflavin biosynthetis operon as found in B. longum NCC 5000. Panel B depicts a heatmap of those genes in closely related strains of B. longum. Genes are colored according to their % identity found by Blast against the NCC 5000 genes.

FIG. 12—In vitro, transitional B. longum transitional strains decrease IL-5 expressed by T helper type 2 skewed cells after 48 hours stimulation to a similar or even greater extent than probiotic B. lactis. NCC5000-5004: B. longum transitional strains, NCC2818: B. animalis subsp. lactis, NCC3089: B. longum subsp. Infantis.

FIG. 13—Peripheral blood mononuclear cells (PBMC) were stimulated for 36 hours in the presence of different bacterial strains including all transitional B longum isolates at 10⁷ CFU/ml and probiotic strains. Cell culture supernatants were collected to assess cytokine expression for IL-10 by ELISA. Standard curve for each cytokine was used to calculate absolute amount (picogram/ml) from optical density readouts. NCC5000-5004: B longum transitional strains, NCC2818: B. animalis subsp. lactis, NCC3089: B. longum subsp. infantis, NCC4007: L rhamnosus and NCC2705: B. longum subsp.longum.

FIG. 14—In-vitro batch fermentations of 3-fucosylactose (3FL) containing infant microbiome with or without the supplementation of Bifidobacterium longum spp infantis or Bifidobacterium longum transitional. Total SCFAs corresponds to the sum of the peak integrals of acetate, butyrate, and propionate. The bar plot indicates the dynamic of consumption and production of total SCFAs between 0 and 24 h (blue bar) and 24 and 48 h (red bar).

### DETAILED DESCRIPTION OF THE INVENTION

All percentages are by weight unless otherwise stated.

The terms “about” or “approximatively” as used herein when referring to a measurable value such as a parameter, an amount, a temporal duration, and the like, are meant to encompass variations of and from the specific value, such as the variation of 1/−10% or less, 1/−5% or less, 1/−1% or less, and +/0.1% or less of and from the specific value, insofar such variations are appropriate to perform in the disclosed invention. It is to be understood that the value to which the modifier “about” or “approximately” refers is itself also specifically, and preferably, disclosed.

The terms “subject”, “individual” and “patient” are used interchangeably to refer to a vertebrate, preferably a mammal, more preferably a human. Mammals include but are not limited to murines, simians, humans, farm animals, sport animals and pets.

The term “infant” means a human subject under the age of 12 months or an age equivalent non-human animal.

The terms “young child” or “toddler” as used herein may mean a human subject aged between 12 months and 5 years of age. Suitably, a “young child” may refer to an age equivalent non-human animal.

The expressions “complementary feeding period”, “complementary period”, “transitional period”, “transitional feeding period” and “weaning period” can be interchangeably used and refer to the period during which the milk, either breast milk or formula, is substituted by other foods in the diet of an infant or a young child. The infant or the young child is typically moved or transitioned gradually from exclusive milk-feeding, either breast feeding or formula feeding, to mixed diet comprising milk and/or solid foods. The transitional period depends on the infant or young child but typically falls between about 4 months and about 18 months of age, such as between about 6 and about 18 months of age, but can in some instances extend up to about 24 months or more. For humans, the weaning period typically starts between 4 and 6 months of age and is considered completed once the infant and/or the young child is no longer fed with breast milk or infant formula, typically at about 24 months of age. In some embodiments, the weaning period is between 4 and 24 months.

The expressions “composition” or “nutritional composition” refer to any kind of composition or formulation that provides a nutritional benefit to an individual and that may be safely consumed by a human or an animal. A nutritional composition may be in solid (e.g. powder), semi-solid or liquid form and may comprise one or more macronutrients, micronutrients, food additives, water, etc. For instance, the nutritional composition may comprise the following macronutrients: a source of proteins, a source of lipids, a source of carbohydrates and any combination thereof. Furthermore, the nutritional composition may comprise the following micronutrients: vitamins, minerals, fiber, phytochemicals, antioxidants, prebiotics, probiotics, bioactives, metabolites (e.g. butyrate, Docosahexaenoic acid (DHA), Eicosapentaenoic acid (EPA), Gamma-Linolenic acid (GLA)) and any combination thereof. The composition may also contain food additives such as stabilizers (when provided in liquid or solid form) or emulsifiers (when provided in liquid form). The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water, e.g. a reconstituted infant formula or follow-on/follow-up formula or infant cereal product or any other formulation designed for infant or young child nutrition). Generally, a nutritional composition can be formulated to be taken enterally, orally, parenterally, or intravenously, and it usually includes one of more nutrients selected from: a lipid or fat source, a protein source, and a carbohydrate source. Preferably, a nutritional composition is for oral use.

In a particular embodiment, the nutritional composition is a “synthetic nutritional composition”. The expression “synthetic nutritional composition” means a mixture obtained by chemical and/or biological means.

The expression “infant formula” as used herein refers to a foodstuff intended for particular nutritional use by infants during the first months of life and satisfying by itself the nutritional requirements of this category of person (Article 2 (c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The expression “growing-up milk” (or GUM) refers to a milk-based drink generally with added vitamins and minerals, that is intended for young children or children.

The terms “fortifier” refers to liquid or solid nutritional compositions suitable for fortifying or mixing with human milk, infant formula, growing-up milk or human breast milk fortified with other nutrients. Accordingly, the fortifier can be administered after dissolution in human breast milk, in infant formula, in growing-up milk or in human breast milk fortified with other nutrients or otherwise it can be administered as a stand-alone composition. When administered as a stand-alone composition, the milk fortifier can be also identified as being a “supplement”.

The term “metabolize” is used herein to mean that a substrate can by broken down, adsorbed and/or utilized by a microorganism. For example, the substrate may promote and/or contribute to the growth and/or survival of the microorganism.

Suitably, the term “capable of metabolizing the glycan substrate” may mean that the B. longum transitional strain encodes at least one CAZyme which is capable of utilizing the glycan substrate. For example, the CAZyme may be capable of catalyzing the hydrolysis of a glycosidic bond within the glycan substrate. Suitably, the B. longum transitional strain may encode at least one, at least two, at least three, at least four or at least five CAZymes that are capable of utilizing the glycan substrate. Suitably, the term “capable of metabolizing the glycan substrate” may mean that the glycan substrate (or a fiber or ingredient comprising the glycan substrate) is capable of promoting growth and/or survival of the B. longum transitional strain (e.g. when added to an anaerobic culture of the B. longum transitional strain). Growth and/or survival of the B. longum transitional strain may be determined by measuring the abundance of 16S rDNA—for example using PCR methods. An illustrative assay for measuring growth of a B. longum transitional strain in the presence of glycan substrates (e.g. in the form of fiber) is provided in the present examples.

Suitably, the glycan substrate is capable of being metabolized by the B longum transitional microorganism. Suitably, the glycan substrate may be capable of promoting growth and/or survival of the B. longum transitional strain. Glycan substrates capable of promoting growth and/or survival of the B. longum transitional strain may be determined by e.g. anaerobic culture of the B. longum transitional strain with the glycan substrate to be tested. Growth and/or survival of the B. longum transitional strain may be determined by measuring bacteria cell number, cell density (e.g. measured by optical density) and/or the abundance of 16S rDNA—for example using PCR methods. An illustrative assay for measuring growth of a B. longum transitional strain in the presence of glycan substrates is provided in the Examples. A glycan substrate capable of promoting growth and/or survival of the B. longum transitional strain may increase the number of B. longum transitional bacteria in an anaerobic culture by at least 20%, at least 30%, at least 40%, at least 50%, at least 75% or at least 100% compared to the number of B. longum transitional bacteria in a control anaerobic culture which does not comprise the HMO. Suitably, a glycan substrate capable of promoting growth and/or survival of the B. longum transitional strain may increase the number of B. longum transitional bacteria in an anaerobic culture by a statistically significant amount (e.g. p-value <0.05 as determined by one-way ANOVA) compared to the number of B. longum transitional bacteria in a control anaerobic culture which does not comprise the glycan substrate.

A “glycan substrate” refers to a glycan that can be metabolized by a microorganism. A glycan substrate may be, for example, a glycoconjugate, oligo- or polysaccharide. Glycoconjugate glycans may comprise N-linked glycans or O-linked glycans within glycoproteins and proteoglycans, or glycolipids. For example, an O-linked glycan may comprise a protein or peptide where the oxygen atom of a serine or threonine residue is linked to a monosaccharide, oligo- or polysaccharide as in the case with glycosaminoglycans (GAGs). Further examples of “glycan substrates” are cellulose, which is a glycan composed of β-1,4-linked D-glucose, and chitin, which is a glycan composed of β-1,4-linked N-acetyl-D-glucosamine. Glycans may be homo- or heteropolymers of monosaccharide residues and can be linear or branched. “Glycan substrate” as used herein encompasses, for example, oligosaccharides and polysaccharides.

The “oligosaccharide” may refer to a carbohydrate that has greater than 2 but relatively few monosaccharide units (typically 3, 4, 5, 6, and up to 10). Exemplary oligosaccharides include, but are not limited to, fructo-oligosaccharides, galacto-oligosaccharides (raffinose, stachyose, verbascose), maltooligosaccharides, gentio-oligosaccharides, cellooligosaccharides, milk oligosaccharides (e.g., those present in secretions from mammary glands), isomalto-oligosaccharides, lactosucrose, mannooligosaccharides, melibiose-derived oligosaccharides, pectic oligosaccharides, xylo-oligosaccharides.

The term “polysaccharide” may refer to a carbohydrate that has more than ten monosaccharide units. Exemplary polysaccharides include, but are not limited to, starch, arabinogalactan, laminarin, chrysolaminarin, xylan, arabinoxylan, mannan, fucoidan and galactomannan. It is to be understood that there is not a precise cut-off or distinction between the terms oligosaccharide and polysaccharide, nor is such a distinction necessary to practice the invention.

The term “glycosaminoglycan” (GAG) or mucopolysaccharide refers to long linear polysaccharides consisting of repeating disaccharide units (i.e. two-sugar units). The repeating two-sugar unit consists of a uronic sugar and an amino sugar, with the exception of keratan, where in the place of the uronic sugar it has galactose. GAGs are classified into four groups based on core disaccharide structures.

“Mucins”, as used herein, may refer to a family of high molecular weight, heavily glycosylated proteins (glycoconjugates). Mucins' key characteristic is their ability to form gels; therefore they are a key component in most gel-like secretions, serving functions from lubrication to cell signaling to forming mechanical and chemical barriers.

The term “HMO” or “HMOs” refers to human milk oligosaccharide(s). These carbohydrates are highly resistant to enzymatic hydrolysis, indicating they may display essential functions not directly related to their caloric value. It has been especially illustrated they play a vital role in the early development of infants and young children, such as the maturation of the immune system. Many different kinds of HMOs are found in the human milk. Each individual oligosaccharide is based on a combination of glucose, galactose, sialic acid (N-acetylneuraminic acid), fucose and/or N-acetylglucosamine with many and varied linkages between them, thus accounting for the enormous number of different oligosaccharides in human milk-over 130 such structures have been identified so far. Almost all of them have a lactose moiety at their reducing end while sialic acid and/or fucose (when present) occupy the terminal position at the non-reducing ends. Depending on the presence of fucose and sialic acid in the oligosaccharide structure, the HMOs can be divided as non-fucosylated (neutral) or fucosylated (neutral) and sialylated (acidic) and non-sialylated molecules, respectively.

The expression “fucosylated oligosaccharide” refers to an oligosaccharide having a fucose residue. It has a neutral nature. Some examples are 2′-fucosyllactose (2-FL), 3-fucosyllactose (3-FL), difucosyllactose (DiFL), lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof. Fucosylated oligosaccharides represents the largest fraction of human milk with 2′-FL constituting up to 30% of the total HMOs. Fucosylated oligosaccharides are thought to reduce the risk of infections and inflammations and to boost growth and metabolic activity of specific commensal microbes reducing inflammatory response.

The expression “N-acetylated oligosaccharide(s)” encompasses both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose), DSLNT (disialyllacto-N-tetraose), and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

The expressions “at least one fucosylated oligosaccharide” and “at least one N-acetylated oligosaccharide” should be understood as “at least one type of fucosylated oligosaccharide” and “at least one type of N-acetylated oligosaccharide”.

The term “sialylated oligosaccharide” refers to an oligosaccharide having a charged sialic acid residue. It has an acidic nature. Some examples are 3′-sialyllactose (3-SL), 6′-sialyllactose (6-SL), sialyllacto-N-tetraose (Lst—e.g. Lst-a, Lst-b or Lst-c).

Suitably, the term “capable of metabolizing the HMO” may mean that the B. longum transitional strain encodes at least one CAZyme which is capable of utilizing the HMO. For example, the CAZyme may be capable of catalyzing the hydrolysis of a glycosidic bond within the HMO. Suitably, the B. longum transitional strain may encode at least one, at least two, at least three, at least four or at least five CAZymes that are capable of utilizing the HMO. Suitably, the term “capable of metabolizing the HMO” may mean that the HMO is capable of promoting growth and/or survival of the B. longum transitional strain (e.g. when added to an anaerobic culture of the B. longum transitional strain). Growth and/or survival of the B. longum transitional strain may be determined by measuring the abundance of 16S rDNA—for example using PCR methods.

The term fibers is used herein to refer to carbohydrates that are indigestible by a human or animal. Such fibers are also discussed in relation to carbohydrates herein. Suitably, the fiber can be fermented by one or more B. longum transitional microorganisms provided in the present use or composition and/or within one or more regions in the gastrointestinal tract within an organism, such as a human or non-human animal. As used herein, the expressions “fiber” or “fibers” or “dietary fiber” or “dietary fibers” within the context of the present invention indicate the indigestible portion, in small intestine, of food derived from plants which comprises two main components: soluble fiber, which dissolves in water and insoluble fiber. Mixtures of fibers are comprised within the scope of the terms above mentioned. Soluble fiber is readily fermented in the colon into gases and physiologically active byproducts and can be prebiotic and viscous. Insoluble fiber does not dissolve in water, is metabolically inert and provides bulking, or it can be prebiotic and metabolically ferment in the large intestine. Chemically, dietary fiber consists of carbohydrate polymers with three or more monomeric units which are not hydrolyzed by endogenous enzymes in the small intestine such as arabinoxylans, cellulose, and many other plant components such as resistant starch, resistant dextrins, inulin, lignin, chitins, pectins, arabinans, arabinogalactans, galactans, xylans, beta-glucans, and oligosaccharides. Non-limiting examples of dietary fibers are: prebiotic fibers such as Fructo-oligosaccharides (FOS), inulin, galacto-oligosaccharides (GOS), fruit fiber, vegetable fiber, cereal fiber, resistant starch such as high amylose corn starch.

As used herein, “added fiber” or “added dietary fiber” indicates an ingredient mainly or totally constituted by fiber which is added to the complementary nutritional composition and whose content in fiber contributes to the total fiber content of the composition. The total fiber content of the complementary nutritional composition is provided by the sum of amount of fiber naturally present in ingredients used in the recipe (for example from whole grain cereal flour) plus amount of added fiber.

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparation or components of microbial cells with a beneficial effect on the health or well-being of the host (Salminen S, Ouwehand A. Benno Y. et al. “Probiotics: how should they be defined” Trends Food Sci. Technol. 1999:10 107-10). The microbial cells according to the present invention are generally bacteria.

The term “cfu” should be understood as colony forming unit.

The “gut microbiota” is the composition of microorganisms (including bacteria, archaea and fungi) that live in the digestive tract.

The term “gut microbiome” may encompass both the “gut microbiota” and their “theater of activity”, which may include their structural elements (nucleic acid, proteins, lipids, polysaccharides), metabolites (signaling molecules, toxins, organic and inorganic molecules) and molecules produced by coexisting hosts and structured by the surrounding environmental conditions (Berg, G., et al., 2020. Microbiome, 8 (1), pp. 1-22).

Bifidobacterium longum Transitional Microorganism

Bifidobacterium longum subsp. microorganisms of a clade that is present in the gut microbiome of the transitional feeding period of mammals, particularly humans, have previously been identified. B. longum microorganisms belonging to this clade are referred to herein as Bifidobacterium longum transitional (B. longum transitional). B. longum transitional strains NCC 5000, NCC 5001, NCC 5002, NCC 5003 and NCC 5004 were deposited with the Collection nationale de cultures de micro-organisms (CNCM), Institute Pasteur by SOCIÉTÉ DES PRODUITS NESTLÉ S.A according to Budapest Treaty on 11th of May 2021 receiving the deposit numbers CNCM I-5683, CNCM I-5684, CNCM I-5685, CNCM I-5686 and CNCM 1-5687, respectively. In US provisional patent application WO2023/278441, it was shown that the B. longum transitional microorganisms are greater in relative abundance during the transitional feeding period (e.g. weaning period) than either B. longum subsp. infantis (B. infantis) or B. longum subsp. longum. Indeed, the relative abundance of B. longum subsp. infantis decreases at the beginning of the transitional feeding period until the end of the transitional feeding period while B. longum subsp. longum begins to increase in abundance. Vatanen et al. demonstrated that this distinct Bifidobacterium longum clade expanded with introduction of solid foods and harbored enzymes for utilizing both breast milk and solid food substrates (Vatanen et al.; 2022, Cell 185, 1-18; published online 1 Nov. 2022; https://doi.org/10.1016/j.cell.2022.10.011).

Suitably, the B. longum transitional microorganism may encode one or more CAZymes selected from the groups recited in Table 1. Suitably, the B. longum transitional microorganism may encode one or two CAZymes selected from the group recited in Table 1.

Suitably, the B. longum transitional microorganism encodes at least one CAZyme selected from the group recited in Table 1 and one or more of the CAZymes selected from the groups recited in Table 2 and 3. For example, the B. longum transitional microorganism may encode at least 2, at least 5, at least 10, at least 20 or at least 30 of the CAZymes selected from the groups recited in Table 2 and 3.

Suitably, B. longum transitional microorganism encodes (i) at least one CAZyme selected from the group recited in Table 1 and (ii) each of the CAZymes recited in Table 3 or each of the CAZymes recited in Table 3 apart from GH5_44.

Suitably, B. longum transitional microorganism encodes (i) at least one CAZyme selected from the group recited in Table 1 and (ii) each of the CAZymes recited in Table 3 or each of the CAZymes recited in Table 3 apart from GH25.

Suitably, the B. longum transitional microorganism does not encode one or more of the CAZymes recited in Table 4. Suitably, the B. longum transitional microorganism does not encode any of the CAZymes recited in Table 4.

The B. longum transitional microorganism of the present invention advantageously harbors genes coding for CAZymes allowing cleavage of sialic residues from glycans such as sialilated oligosachharides, glycoproteins and glycolipids. This allows effective utilization of the sialylated oligosaccharides that are present in the breast milk at weaning and hence can participate to an appropriate development of the gut microbiome of an infant and/or a young child. It may also help in preventing presence of enteropathogens.

In some embodiments, a B. longum transitional microorganism comprises a sialidase or neuraminidase family 33 (GH33, sialidase or neuraminidase) gene having at least 60% identity with BLON_2348 gene present in B. longum subsp. infantis ATCC 15697.

In some embodiments, a B. longum transitional microorganism comprises sialidase or neuraminidase family 33 (GH33, sialidase or neuraminidase) gene having about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% identity with BLON_2348 gene present in B. longum subsp. infantis ATCC 15697.

In some embodiments, a B. longum transitional microorganism comprises sialidase or neuraminidase family 33 (GH33, sialidase or neuraminidase) gene having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity with BLON_2348 gene present in B. longum subsp. infantis ATCC 15697.

In some embodiments, the B. longum transitional microorganism used according to the present invention comprises a glycosyl hydrolase family 95 (GH95, α-L-galactosidase; α-L-fucosidase; α-1,2-L-fucosidase) gene having at least 60% of identity with BLON_2335 gene present in B. longum subsp. infantis ATCC 15697 and/or a glycosyl hydrolase family 29 (GH29, α-L-fucosidase; α-1,3/1,4-L-fucosidase; α-1,2-L-fucosidase) having at least 60% identity with BLON_2336 gene present in B. longum subsp. infantis ATCC 15697.

In some embodiments, the B. longum transitional microorganism used according to the present invention comprises a sialidase or neuraminidase family 33 (GH33, sialidase or neuraminidase) gene having at least 60% identity with BLON_2348 gene present in B. longum subsp. infantis ATCC 15697.

In some embodiments, the B. longum transitional microorganism preferentially utilizes 3-fucosyllactose (3-FL) over 2′-fucosyllactose (2′-FL). Suitably, B. longum transitional microorganism may preferentially utilize 3-FL over 2′-FL in a ratio between 0.1:5, preferably in a ratio between 0.1:4, more preferably in a ratio between 0.2:2.

In some embodiments, the B. longum transitional microorganism used in the present invention utilizes 3-FL more efficiently than 2′-FL, as demonstrated by a better growth, for example as shown in the present Examples. In some embodiments, a B. longum transitional microorganism has an Average Nucleotide Identity (ANI) of at least 98% with at least one B. longum strain selected in the group consisting of CNCM I-5683, CNCM I-5684, CNCM I-5685, CNCM 1-5686, CNCM 1-5687 and CMCC-P0001 (ATCC BAA-2753), and any combination thereof. Suitably, the B. longum transitional microorganism encodes one or more CAZymes selected from the group recited in Table 1 and has an ANI of at least 98% with at least one B. longum strain selected in the group consisting of CNCM I-5683, CNCM I-5684, CNCM I-5685, CNCM I-5686, CNCM I-5687 and CMCC-P0001 (ATCC BAA-2753), and any combination thereof. Suitably, the B. longum transitional microorganism encodes one or more GH31 CAZymes and has an ANI of at least 98% with at least one B. longum strain selected in the group consisting of CNCM I-5683, CNCM I-5684, CNCM I-5685, CNCM I-5686, CNCM I-5687 and CMCC-P0001 (ATCC BAA-2753), and any combination thereof. In some embodiments, a B. longum transitional microorganism has an ANI of about 98% to 100% with at least one B. longum strain selected in the group consisting of CNCM I-5683, CNCM I-5684, CNCM I-5685, CNCM I-5686, CNCM I-5687 and CMCC-P0001 (ATCC BAA-2753), and any combination thereof. In some embodiments, a B. longum transitional microorganism has an ANI of at least 98%, 98.1%, 98.2%, 98.3%, 98.4%, 98.5%, 98.6%, 98.6%, 98.7%, 98.8%, 98.9%, 99%, 99.1%, 99.2%, 99.3%, 99.4%, 99.5%, 99.6%, 99.7%, 99.8%, 99.9%, or 100% with at least one B. longum strain selected in the group consisting of CNCM I-5683, CNCM I-5684, CNCM I-5685, CNCM I-5686, CNCM I-5687 and CMCC-P0001 (ATCC BAA-2753), and any combination thereof. In some embodiments, a B. longum transitional microorganism has an ANI of at least 98.6%, of at least 98.6%, of at least 98.7%, of at least 98.8%, of at least 98.9%, of at least 99%, of at least 99.1%, of at least 99.2%, of at least 99.3%, of at least 99.4%, of at least 99.5%, of at least 99.6%, of at least 99.7%, of at least 99.8%, of at least 99.9% or of at least 100% with at least one B. longum strain selected in the group consisting of CNCM I-5683, CNCM I-5684, CNCM I-5685, CNCM I-5686, CNCM 1-5687 and CMCC-P0001 (ATCC BAA-2753), and any combination thereof.

Methods for sequencing microbial genomes are well known in the art (see e.g. Segerman; Front. Cell. Infect. Microbiol.; 2020; 10; Article 527102 & Donkor; Genes; 2013; 4 (4); 556-572). By way of example, metagenomics methods may be used. Suitable metagenomics methods may be performed using shotgun sequencing data, for example. Suitable metogenomics methods are known in the art and include MetaPhlAn 3.0, for example (see Beghini et al.; eLife 2021; 10: e65088; https://huttenhower.sph.harvard.edu/metaphlan).

The “Average Nucleotide Identity (ANI)” is a term of art that refers to a distance-based approach to delineate species based on pair-wise comparisons of their genome sequences and is an in silico alternative to the traditional DNA-DNA hybridization (DDH) techniques that have been used for phylogenetic definition of a species (Goris et al., 2007, “DNA-DNA hybridization values and their relationship to whole-genome sequence similarities”, Int. J. Syst. Evol. Microbiol. 57:81-91). Based on DDH, strains with greater than 70% relatedness would be considered to belong to the same species (see e.g., Wayne et al., 1987, Report of the Ad-Hoc-Committee on Reconciliation of Approaches to Bacterial Systematics. Int J Syst Bacteriol 37:463-464). ANI is similar to the aforementioned 70% DDH cutoff value and can be used for species delineation. ANI has been evaluated in multiple labs and has become the gold standard for species delineation (see e.g., Kim et al., 2014, “Towards a taxonomic coherence between average nucleotide identity and 16S rRNA gene sequence similarity for species demarcation of prokaryotes”, Int. J. Syst. Evol. Micr. 64:346-351; Richter et al., 2009, “Shifting the genomic gold standard for the prokaryotic species definition”, P Natl Acad Sci USA 106:19126-19131; and Chan et al., 2012, “Defining bacterial species in the genomic era: insights from the genus Acinetobacter”, Bmc. Microbiol. 12)).

The ANI of the shared genes between two strains is known to be a robust means to compare genetic relatedness among strains, and that ANI values of about 95% correspond to the 70% DNA-DNA hybridization standard for defining a species. See, e.g., Konstantinidis and Tiedje, Proc Natl Acad Sci USA, 102 (7): 2567-72 (2005); and Goris et al., Int Syst Evol Microbiol. 57 (Pt 1): 81-91 (2007). The ANI between two bacterial genomes is calculated from pair-wise comparisons of all sequences shared between any two strains and can be determined, for example, using any of a number of publicly available ANI tools, including but not limited to OrthoANI with usearch (Yoon et al. Antonie van Leeuwenhoek 110:1281-1286 (2017)); ANI Calculator, JSpecies (Richter and Rossello-Mora, Proc Natl Acad Sci USA 106:19126-19131 (2009)); and JSpeciesWS (Richter et al., Bioinformatics 32:929-931 (2016)). Other methods for determining the ANI of two genomes are known in the art. See, e.g., Konstantinidis, K. T. and Tiedje, J. M., Proc. Natl. Acad. Sci. U.S.A., 102:2567-2572 (2005); and Varghese et al., Nucleic Acids Research, 43 (14): 6761-6771 (2015). In a particular embodiment, the ANI between two bacterial genomes can be determined, for example, by averaging the nucleotide identity of orthologous genes identified as bidirectional best hits (BBHs). Protein-coding genes of a first genome (Genome A) and second genome (Genome B) are compared at the nucleotide level using a similarity search tool, for example, NSimScan (Novichkov et al., Bioinformatics 32 (15): 2380-23811 (2016)). The results are then filtered to retain only the BBHs that display at least 70% sequence identity over at least 70% of the length of the shorter sequence in each BBH pair. The ANI of Genome A to Genome B is defined as the sum of the percent identity times the alignment length for all BBHs, divided by the sum of the lengths of the BBH genes. These and ANI determination techniques are known in the art.

Suitably, a B. longum transitional microorganism selected from the group consisting of CNCM I-5683, CNCM I-5684, CNCM I-5685, CNCM I-5686, CNCM I-5687 and CMCC-P0001 (ATCC BAA-2753), represents the reference genome to which a microbial genome is compared.

Genome sequences for B. longum transitional strains NCC 5000 (CNCM I-5683), NCC 5001 (CNCM I-5684), NCC 5002 (CNCM I-5685), NCC 5003 (CNCM 1-5686) and NCC 5004 (CNCM I-5687) are available via Joint Genome Project (JGI) Study number: Gs0156595 (https://genome.jgi.doe.gov/portal/). Analysis project numbers and taxon numbers for each genome are as follows:

| Strain | JGI analysis project | JGI taxon |
|---|---|---|
| B. longum NCC 5000 | Ga0527908 | 2951181949 |
| B. longum NCC 5001 | Ga0529016 | 2951184202 |
| B. longum NCC 5002 | Ga0529017 | 2951186501 |
| B. longum NCC 5003 | Ga0529018 | 2951188792 |
| B. longum NCC 5004 | Ga0529019 | 2951191018 |

In some embodiments, the B. longum transitional microorganism for use in the present invention is isolated from a human.

In some other embodiments, the B. longum transitional microorganism is not of the subspecies B. longum subsp. longum or B. longum subsp. infantis.

Suitably, the B. longum transitional microorganism is provided as a probiotic. Suitably, the B. longum transitional microorganism is provided in a composition.

Treating and/or Preventing an Allergy and/or Allergic Sensitization

‘Allergy’, as used herein, may refer to an allergic disorder or an allergic reaction (including symptoms thereof).

The B. longum transitional microorganism and/or prebiotic may be for use treating and/or preventing an allergy and/or allergic sensitization in an infant or young child.

“Treating”, as used herein, may refer to administering the B. longum transitional microorganism and/or prebiotic to a subject having an existing disease or condition in order to lessen, reduce or improve at least one symptom associated with the disease and/or to slow down, reduce or block the progression of the disease.

“Preventing”, as used herein, may refer to administering the B. longum transitional microorganism and/or prebiotic to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease. Suitably, the B. longum transitional microorganism and/or prebiotic may be administered to a subject in order to reduce the likelihood of the infant or young child developing an allergy and/or allergic sensitization.

Allergic sensitization in childhood, especially in early childhood and especially to food allergens, is critical and development of an “allergic phenotype” or “atopy” has been shown to facilitate subsequent sensitization to other allergens. Hence allergies in childhood can be the first step of an allergic cascade leading to multiple allergies later in life, a process commonly referred to as the “Atopic March”. For example, children with persistent food hypersensitivity early in life have a dramatically increased risk to develop allergic rhinitis (hay fever) or asthma later in childhood (Ostblom, E. et al. (2008); Phenotypes of food hypersensitivity and development of allergic diseases during the first 8 years of life, Clinical and Experimental Allergy, 38 (8): 1325-1332). Children with milder forms of food hypersensitivity also have increased risk for development of respiratory allergies but to a lesser degree than children with persistent food hypersensitivity. Therefore, attenuating the severity of food hypersensitivity may be crucial for slowing down the “Atopic March”. In this context the management of allergic episodes and prevention of allergies are, in childhood and infancy, of the highest importance.

In one embodiment preventing and/or reducing the risk of developing an allergy and/or allergic sensitization is by primary prevention. “Primary prevention” is the effect of preventing or reducing the risk of sensitization of patients to allergens, characterized by absence or reduced levels of allergen-specific IgE antibodies. Preventing or reducing sensitization may result in absence or reduction of allergic symptoms upon exposure to the same allergen. By modulating the way a patient gets sensitized in regard to one allergen or one group of allergens (primary prevention), the subsequent allergic response may also be modulated.

Food allergens are among the first allergens that infants encounter in their early life: typically, cow's milk proteins may be encountered by infants not receiving exclusive breast-feeding. Milk-proteins are indeed among the most frequently observed causes for food allergy in infancy, followed by eggs and wheat proteins. In general, food allergies can manifest in cutaneous (rash, eczema, others) and gastrointestinal symptoms (abdominal cramps; pain, especially in the abdomen; vomiting) in infants and young children. Food allergies are the most common trigger of severe allergic reactions, which may lead to life-threatening anaphylaxis.

Further sensitization and episodes of allergies can also appear when the infant/young child is exposed to a novel food such as cereals, vegetables, fruits, nuts or fish, and also to air-borne allergens such as pollen, house dust mites and animal dander. Adults are affected to a large extent by contact and respiratory allergies. Recent data from the WHO (Clark, M. J. and. Million, R. P (2009) Allergic rhinitis: market evolution, Nature Reviews, Drug Discovery, 8, p. 271-272) indicates that up to 30-40% of the world's population suffer from some form of respiratory allergy.

Animals, particularly small animals such as pets- and especially companion animals such as dogs and cats, may also suffer from food allergies and food intolerances, as well as environmental allergens. These typically manifest in similar symptoms to humans, e.g. gastrointestinal disturbances such as diarrhoea, vomiting and abdominal discomfort, and also dermatitis or pruritis. In small animals, particularly dogs, the most frequent cause of chronic diarrhoea is food-responsive enteropathy (diet-responsive enteropathy or food-responsive diarrhoea).

By treating and/or preventing or reducing the risk of sensitization of subjects to allergens the compounds and compositions of the present invention may be used for preventing or treating food allergies, respiratory allergies and dermatological allergies.

In some embodiments an allergic response is a specific IgE-associated immune response and/or a T cell-dependent hypersensitive reaction. Thus, in some embodiments treating and/or preventing and/or reducing the risk of developing an allergy and/or allergic sensitization comprises reducing or preventing specific IgE-associated immune responses and/or a T cell-dependent hypersensitive reaction. In some embodiments allergic inflammation is reduced and/or tolerance (e.g. oral tolerance) is enhanced.

Suitably, the allergic disorder is selected from one or more of the group consisting of: a food allergy, a respiratory allergy and a dermatological allergy.

In one embodiment the allergic disorder is selected from one or more of the group consisting of: rhinitis, asthma, dermatitis, atopic dermatitis, contact dermatitis, eczema, atopic eczema, urticaria, psoriasis, eosinophilic oesophagitis and an eosinophilic-associated gastrointestinal disease.

In one embodiment the allergen in the allergic disorder is selected from one or more of: a food allergen, dust mite, pollen, molds or mold spores, weed pollen, tree pollen, grass pollen, fleas, pet hair, feathers, pollution or pet dander.

In one embodiment the allergen in the allergic disorder is a food allergen. Suitably, wherein the food allergen is selected from: a nut, tree nut, peanut, fish, shellfish, molluscs, crustaceans, milk, egg, soy, gluten, cereals, wheat, oats, barley, rye, celery, corn, lupin, sulphites, sesame, mustard, rice, poultry and meat.

In one embodiment the allergen is an aeroallergen. Suitably, wherein the aeroallergen is selected from dust mite, pollen, moulds or mold spores, weed pollen, tree pollen, grass pollen, fleas, pet hair, feathers, pollution or pet dander.

Suitably, treating, preventing or reducing the risk of an allergy and/or allergic sensitization may refer to reducing or ameliorating one or more symptoms as described herein.

A “food allergy” as used herein refers to an abnormal immune response to one or more food allergens, typically an IgE reaction caused by the release of histamine but also encompassing non-IgE immune responses. Symptoms of food allergy may include itchiness, swelling of the tongue, vomiting, diarrhea, hives, trouble breathing, or low blood pressure. When the symptoms are severe, it is known as anaphylaxis.

As used herein, the term “food allergen” refers to proteins or derivatives thereof that cause abnormal immune responses. Purified food allergens may be named using the systematic nomenclature of the Allergen Nomenclature Sub-Committee of the World Health Organization and International Union of Immunological Societies. Allergen names are composed of an abbreviation of the scientific name of its source (genus: 3-4 letters; species: 1-2 letters) and an Arabic numeral, for example Der p 1 for the first allergen to be described from the house dust mite Dermatophagoides pteronyssinus. Food allergens are derived from proteins with a variety of biologic functions, including proteases, ligand-binding proteins, structural proteins, pathogenesis-related proteins, lipid transfer proteins, profilins, and calcium-binding proteins.

A list of food allergens is provided on the official website of the WHO/IUIS Allergen Nomenclature Database, http://www.allergen.org/index.php. (Radauer, C., et al., 2014. Allergy, 69 (4), pp. 413-419 and Pomés, A., et al., 2018. Molecular immunology).

A “respiratory allergy” or “aeroallergen” as used herein refers to an abnormal immune response to one or more airborne allergens. Airborne allergens may include pollen, molds or mold spores, weed pollen, tree pollen, grass pollen, and dander. Respiratory allergies may include for example allergic rhinitis and allergic asthma. Symptoms of allergic rhinitis (hay fever) include a runny or stuffy nose, sneezing, red, itchy, and watery eyes, and swelling around the eyes. Symptoms of allergic asthma include episodes of wheezing, coughing, chest tightness, and shortness of breath.

A “dermatological allergy” as used herein refers to an abnormal immune response caused by contact with one or more environmental allergens. Environmental allergens may include a food allergen, dust mite, pollen, moulds or mold spores, weed pollen, tree pollen, grass pollen, fleas, pet hair, feathers or pet dander. Dermatological allergies may include for example dermatitis, atopic dermatitis, contact dermatitis, eczema, atopic eczema, urticaria, and psoriasis. These are typically a group of diseases that results in inflammation of the skin and symptoms include itchiness, red skin and a rash.

Allergic disorders may also include other allergic inflammatory conditions, for example eosinophilic oesophagitis and an eosinophilic-associated gastrointestinal disease. Eosinophilic esophagitis is an allergic inflammatory condition of the esophagus that involves eosinophils, a type of white blood cell. Symptoms are swallowing difficulty, food impaction, vomiting, and heartburn.

Suitably, the present B. longum transitional microorganism and/or prebiotic may increase the levels of an anti-inflammatory cytokine(s) in the infant or young child. Suitably, the B. longum transitional microorganism and/or prebiotic may increase the levels of IL-10 in the infant or young child. IL-10 is a critical immune regulatory cytokine secreted by several immune cells including regulatory T cells and has been shown to dampen pro-inflammatory IL-12p40/T helper type 1 mediated responses. This cytokine is critical in the establishment and maintenance of intestinal homeostasis between host immune cells and gut microbiota. Consequently, aberrant IL-10 signaling has been reported in chronic inflammatory intestinal disorders such as inflammatory bowel disease (IBD). Changes in the intestinal microbiota at a time of weaning induces a strong immune reaction coupled with an induction of regulatory immune responses (weaning reaction). Disturbance of this weaning reaction may increase susceptibility to immune pathologies (such as allergic disorders) later in life.

Suitably, the B. longum transitional microorganism and/or prebiotic may increase the IL-10/IL-12 ratio of the infant or young child.

Suitably, the B. longum transitional microorganism and/or prebiotic may decrease the levels of proinflammatory cytokines, particularly cytokines associated with allergy and/or the atopic response. For example the B. longum transitional microorganism and/or prebiotic may decrease the levels of IL-5 in the infant or young child. IL-5 is an interleukin produced by type-2 T helper cells and mast cells, it stimulates B cell growth and increases immunoglobulin secretion-primarily IgA. It is also a key mediator in eosinophil activation. IL-5 is associated with the cause of several allergic diseases including allergic rhinitis and asthma. Reducing IL-5 levels has been shown reduce allergic symptoms in diseases such as severe asthma, for example (Casale et al.; 2021; Annals of Allergy, Asthma & Immunology; 127 (3); 354-362 and Sposato et al.; 2021; Int Arch Allergy Immunol; 182 (4): 311-318).

The cytokine effects mediated by the present probiotic and/or prebiotic may be systemic. As such, the cytokine effects (e.g. increase in the levels of IL-10, increase in the IL-10/IL-12 ratio and/or decrease in IL-5) may systemically reduce the risk of developing an allergy and/or allergic sensitization and/or promote immune tolerance as described herein. The cytokine effects may occur locally in the gut, the lungs and/or the skin of the infant or young child. Suitably, the cytokine effects may occur in the gut of the infant or young child. Accordingly, the cytokine effects may reduce the risk of developing an allergy and/or allergic sensitization and/or promote immune tolerance in a particular organ or system.

Suitably, the B. longum transitional microorganism and/or prebiotic may modulate the permeability of the gut epithelial barrier of the infant or young child. Suitably, the B. longum transitional microorganism and/or prebiotic may decrease the permeability of the gut epithelial barrier. Increased permeability of the gut epithelial barrier may be associated with an increase crossing of e.g. haptens and antigens across the intestinal epithelium.

Commensal gastrointestinal microbes constitute the earliest and most substantial stimulus for the development of the gut associated lymphoid tissue and associated immune system.

There is evidence from epidemiological studies that Western-type living conditions, e.g. reduced consumption of fermented food, substantial use of antibiotics and other drugs, and increased hygiene, are associated with the rise in allergic diseases. This links to the so-called “hygiene hypothesis” that suggests that a lack of exposure to microbial stimulus early in childhood is a major factor effecting the prevalence of allergic diseases. Indeed, epidemiological studies have demonstrated an association between the development of allergic diseases and disturbance of the gastrointestinal microbiota. Epidemiological data show that atopic children have different intestinal flora compared with non-atopic children.

Such changes in the intestinal flora may also have a negative impact on the integrity of the intestinal barrier. Impaired barrier function, termed “leaky gut” has long been considered a predisposing factor for gastrointestinal diseases (Heyman M., Eur. J. Gastroenterol. Hepatol. 17:1279-1285; Odenwald M., Nature Reviews Gastroenterology & Hepatology, (2017), (14), 9 21). As such, alterations in gut barrier integrity/function have multiple consequences facilitating the onset of numerous diseases depending on other hits and on genetic and epigenetic constellations. Food allergy patients often demonstrate with increased intestinal permeability, which correlates with the severity of their clinical symptoms (Ventura, M. T et al. 2006. Dig. Dis. Sci. 38:732-736). Preclinical animal models further provide corroborative evidence supporting a role for intestinal barrier dysfunction and leaky gut, predisposing to oral sensitization and subsequent development of food allergy. In addition, Western diet-induced alterations in intestinal permeability promote food allergen sensitization and clinical allergy symptoms in mice in response to dietary antigens (Hussain M. et al. J. Allergy Clin. Immunol. (2019). Probiotics represent one nutritional attempt to improve/reinforce intestinal barrier integrity and/or function (Ewaschuk J B et al., Am J Physiol Gastrointest Liver Physiol. 2008 November; 295 (5): G1025-34). Reinforcing intestinal barrier integrity by means of probiotic supplementation may thus prevent sensitization to oral allergens in at risk individuals. (Tulyeu J, Microorganisms. 2019 Oct. 16; 7 (10). Besides an established role of gut barrier function in allergen sensitization, uncontrolled immune responses towards dietary or environmental antigens foster the development of type-2 immune mediated allergic disorders. Probiotic cultures or mixes of probiotics have well known immunomodulatory properties that can prevent or alleviate allergic responses.

The epithelial barrier of human new-borns is not fully mature at birth. Transfer of macromolecules or antigens across the intestinal epithelium of an infant or young child induces differentiation of regulatory T-cells (Tregs) and is essential for the induction of tolerance and protection from allergic diseases. However, the route of transepithelial passage of antigens is important in determining the immune response/outcome. Transcellular passage with enterocyte processing may be preferable compared to uncontrolled paracellular passage (leakage), which instead might result in inflammation and/or sensitization.

As demonstrated in the present Examples, B longum transitional microorganisms encode genes implicated in the production of riboflavin and folic acid.

Riboflavin (vitamin B2) is an essential metabolite for the host physiological processes. The mammalian host is not capable of producing riboflavin and therefore strictly relies on external supply from the diet and from the gut microbiome production. Once converted into an active form (FAD and FMN), riboflavin is involved in multiple metabolic pathways including energy metabolism, fatty acid oxidation and purine catabolism

Preclinical and clinical studies on acute and chronic riboflavin deficiency resulted in mucous membrane lesions (EFSA Journal. 2010; 8 (10): 1814.). The gastrointestinal tract harbours one of the largest mucosal epithelia that form a direct barrier allowing the absorption of nutrients and immune sensing, while limiting the transport of potentially harmful antigens and microorganisms. Riboflavin was found to maintain structure and function of small intestine including size and cellularity of duodenal crypts (Yates et al, Br J Nutr. 2001; 86 (5): 593-9) and villus length and number (Williams et al, Gut. 1996; 39:220-225) in rat models. Yates et al, also showed the importance of luminal riboflavin in rat early post-natal development of the gastrointestinal tract as the absence of luminal riboflavin resulted in crypt hypertrophy and reduced crypt bifurcation even when systemic riboflavin status was maintained via injection (Yates et al, Dig Dis Sci 2003; 48 (6): 1159-64.). Riboflavin depletion affects the proliferation and proliferative potential of intestinal cells in adult humans, which may impair gastrointestinal function (Nakano et al, Dig Dis Sci 2011; 56 (4): 1007-19.). These results highlight the role of riboflavin to support the normal development or maintenance of mucosal barrier including the gastro-intestinal mucosa. B longum transitional microorganism may therefore maintain gut epithelial integrity during the transition from breast milk to solid food through riboflavin production, thus promoting a healthy imprinting of the immune system.

Folic acid (vitamin B9) is another essential vitamin for the synthesis of nucleic acid and protein synthesis and inadequate level of folic acid has been linked with altered immune response. In particular, the folate receptor 4 is highly expressed in regulatory T (Treg) cells, and folate participate in the maintenance of Treg cells survival (Kunisawa et al, 2013, Front Immunol 4:189). Mice fed with folate-deficient diet have decreased number of intestinal Treg cells leading to an increase in susceptibility of inflammation (Kunisawa et al, 2012; PLOS One 7 (2): e32094). Treg are essential in controlling the pro-allergic TH2 cells response as well as producing the anti-inflammatory IL-10. The B. longum transitional microorganism may influence the tolerogenic immune responses via the production of folate.

The present invention also provides the use of a prebiotic as described herein to increase the levels of riboflavin and/or folic acid in the gut of an infant or child; wherein the riboflavin and/or folic acid is increased as a result of the prebiotic promoting the growth and/or survival of a B. longum transitional microorganism in the gut of the infant or child.

The present invention further provides a method of increasing the level of riboflavin and/or folic acid in the gut of an infant or child; wherein the method comprises administering a prebiotic as described herein to the infant or child in order to promote the growth and/or survival of a B. longum transitional microorganism in the gut of the infant or child.

The B. longum transitional microorganism and/or prebiotic may promote immune tolerance in the infant and/or young child. For example, the B. longum transitional microorganism and/or prebiotic may promote immune tolerance in the gut of the infant and/or young child. Immune tolerance may be promoted by a mechanism as described herein—e.g. increased IL-10 production, decreased IL-5 production and/or reduced permeability of the gut epithelial barrier.

### Prebiotic

The invention further provides a prebiotic for use in treating and/or preventing an allergy and/or allergic sensitization in an infant or young child by promoting the growth of a Bifidobacterium longum transitional microorganism in the gut of the infant or young child, wherein the prebiotic is:
i. a glycan substrate, suitably selected from the group recited in any of Tables 1 to 3; and/or
ii. a human milk oligosaccharide (HMO), suitably selected from the group consisting of 2′-O-fucosyllactose (2′-FL), 3′-O-fucosyllactose (3′-FL), lactodifucotetraose/difucosyllactose (di-FL), 3′-O-sialyllactose (3′-SL), 6′-O-sialyllactose (6′-SL), lacto-N-tetraose (LNT) and lacto-N-neotetraose (LNnT) and any combination thereof.

In another aspect, the invention provides a prebiotic for use in treating and/or preventing an allergy and/or allergic sensitization in an infant or young child by promoting the growth of a Bifidobacterium longum transitional microorganism in the gut of the infant or young child.

### Glycan Substrate/Carbohydrate-Active Enzymes (CAZymes)

The B. longum transitional microorganisms encode a profile of Carbohydrate-Active Enzymes (CAZymes). Without wishing to be bound by theory, it is considered that targeting these CAZymes by, for example, providing suitable glycan substrates in the form of a prebiotic, may promote the growth and/or survival of the Bifidobacterium longum transitional microorganisms in the gut microbiota of an infant or young child.

Suitably, promoting the growth and/or survival of the B. longum transitional microorganism may refer to increasing the number and/or concentration of the B. longum transitional microorganism in the gut microbiota.

In particular, the CAZymes encoded by each of the Bifidobacterium longum transitional strains NCC 5000, NCC 5001, NCC 5002, NCC 5003 and NCC 5004, which were deposited with the Institute Pasteur according to Budapest Treaty on 11th of May 2021 receiving the deposit numbers CNCM I-5683, CNCM I-5684, CNCM 1.5685, CNCM I-5686 and CNCM I-5687, respectively; have been determined.

Carbohydrate-active enzymes (CAZymes) are responsible for the synthesis and breakdown of glycoconjugates, oligo- and polysaccharides. They typically correspond to 1-5% of the genes in the living organism. Glycoconjugates, oligo- and polysaccharides play essential roles in many biological functions, for example as structure and energy reserve components and in many intra- and intercellular events. The Carbohydrate Active Enzyme (CAZy) classification is a sequence-based family classification system that correlates with the structure and molecular mechanism of CAZymes (www.cazy.org).

CAZymes include glycoside hydrolyases (GH), glycosyltransferases (GT), polysaccharide lyases (PL), carbohydrate esterases (CE), and carbohydrate-binding module families (CBM)

Suitably, the CAZyme may be a glycoside hydrolyase (GH). GHs catalyze the hydrolysis of glycosidic bonds between two or more carbohydrates or between a carbohydrate and a non-carbohydrate moiety. In most cases, the hydrolysis of the glycosidic bond is catalyzed by two amino acid residues of the enzyme: a general acid (proton donor) and a nucleophile/base. Depending on the spatial position of these catalytic residues, hydrolysis occurs via overall retention or overall inversion of the anomeric configuration.

A GH classification system is provided by the CAZy classification. Herein, GHs are divided into families based on molecular function (e.g., GH1, GH2, GH3, GH4, etc.). These families are then further divided into subfamilies based on subgroups found within a family that share a more recent ancestor and, typically more uniform in molecular function (e.g., GH13_1,

GH13_2, GH13_3, GH13_4, etc.).

Table 1 provides details of CAZymes that are unique to the Bifidobacterium longum transitional strains (i.e., not encoded by Bifidobacterium longum suis/suillum, Bifidobacterium longum longum or Bifidobacterium longum infantis strains). Table 1 also provides a summary of the glycan substrate metabolized by each CAZyme and illustrative dietary fiber sources/ingredients.

TABLE 1

| | | | | | Example of |
|---|---|---|---|---|---|
| | | | Dietary | | commercially |
| | | Glycan | fiber | Potential fibre rich | available |
| CAZyme | Function | substrate | source | ingredient (targeted fibre) | purified fibre |
| GH13_14 | Pullulanase, α- | α-1,4(α-1,6)(α- | Fungi, | Filamentous fungus, corn, | Pullulan from |
| | amylase, α- | 1,3)-glucose | resistant | dextrin, polydextrose | Aureobasidium |
| | glucosidase | (pullulan, | starch from | | pullulans, |
| | | dextrin, | cereals, | | polydextrose, |
| | | amylose, | (whole | | high amylose |
| | | amylopectin) | grains), | | starch, |
| | | | legumes, | | resistant |
| | | | vegetables | | dextrin |
| | | | and roots | | |
| GH13_32 | α-amylase and | Linear α-1,4- | Resistant- | Corn, dextrin | High amylose |
| | maltotriose- | glucan | starch from | | starch, |
| | producing α- | (amylose and | cereals | | resistant |
| | amylase. | amylopectin) | (whole | | dextrin |
| | | | grains), | | |
| | | | legumes, | | |
| | | | vegetables | | |
| | | | and roots | | |
| GH30_2 | β-xylosidase. | β-1,4-xylose | Cereals | Wheat, rye, barley, oat, rice, | Wheat |
| | | (arabinoxylan, | (whole | corn, and sorghum (whole | arabinoxylan, |
| | | xylan) | grains) | grains) | corn |
| | | | | | arabinoxylan, |
| | | | | | psyllium husk |
| | | | | | arabinoxylan, |
| | | | | | XOS |
| GH8 | Chitosanase, | β-1,4- | Crustacean | Chitooligosaccharides and | Chitosan, |
| | cellulase, | glucosamine | shells, fungi, | chitosan, wheat, rye, barley, | barley bran |
| | licheninase, | (chitosan), β- | cereals | oat, rice, corn, and sorghum | fiber, oat fiber |
| | endo-β-1,3(4)- | 1,3(β-1,4)- | (whole | (whole grains) | |
| | glucanase, | glucan, β-1,4- | grains) | | |
| | endo-1,4-β- | xylan | | | |
| | xylanase, and | | | | |
| | reducing-end- | | | | |
| | xylose | | | | |
| | releasing exo- | | | | |
| | oligoxylanase. | | | | |
| GH35 | β- | β-1,4(β-1,3)- | Fruit and | Carrot, radish, tomato, | Carrot pectin, |
| | galactosidase, | galactose | vegetable | barley, soybean, peach, | larch |
| | exo-β- | (arabinogalactan | pectins, | pear, banana, sea weed | arabinogalactan, |
| | glucosaminidase, | and | cereals | | gum arabic, |
| | exo-β-1,4- | galactan), β- | (whole | | tomato fiber, |
| | galactanase, | 1,3-galactose | grains), or | | carrageenan |
| | and β-1,3- | (agarans), α- | sea weed | | |
| | galactosidase. | 1,3(β-1,4)- | dietary | | |
| | | galactose (β- | fiber | | |
| | | carrageenan) | | | |
| GH50 | β-agarase. | β-1,3- | Sea weed | Red algae | |
| | | galactose | dietary | (Palmaria palmata) | |
| | | (agarans) | fiber | | |

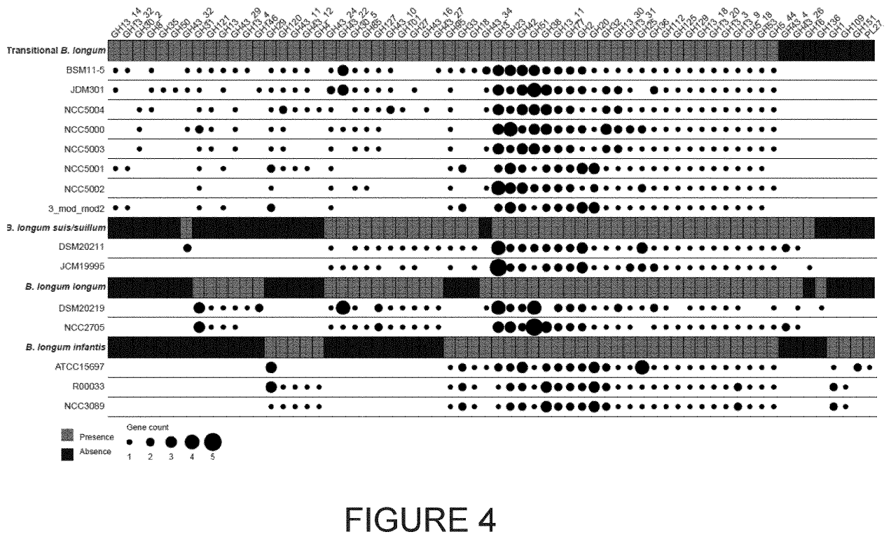

Table 2 provides details of CAZymes that were present in at least one Bifidobacterium longum transitional strain but not present in at least one of the groups selected from the Bifidobacterium longum subsp. suis/suillum, Bifidobacterium longum subsp. longum or Bifidobacterium longum subsp. infantis strains presented in FIG. 4. Table 2 also provides a summary of the glycan substrate metabolized by each CAZyme and illustrative dietary fiber sources/ingredients.

TABLE 2

| | | | | Potential | Example of |
|---|---|---|---|---|---|
| | | | | fibre rich | commercially |
| | | | | ingredient | available |
| | | Glycan | Dietary fiber | (targeted | purified |
| CAZyme | Function | substrate | source | fibre) | fibre |
| GH43_32 | β-D- | β-1,4(β-1,3)- | Fruit pectins and | Carrot, radish, | Carrot pectin, larch |
| | galactofuranosidase. | galactose | cereals (whole | tomato, barley, | arabinogalactan, |
| | | (arabinogalactan) | grains) | soybean, peach, | Gum arabic |
| | | | | pear, banana | |
| GH31 | α-glucosidase, α- | Linear α-1,4- | Resistant-starch | Corn, dextrin | High amylose |
| | galactosidase, α- | glucan | from cereals | | starch, resistant |
| | mannosidase, α- | (amylose and | (whole grains), | | dextrin |
| | 1,3-glucosidase, | amylopectin) | legumes, | | |
| | sucrase-isomaltase, | | vegetables and | | |
| | α-xylosidase, α- | | roots | | |
| | glucan lyase, | | | | |
| | isomaltosyltransferase, | | | | |
| | oligosaccharide | | | | |
| | α-1,4- | | | | |
| | glucosyltransferase, | | | | |
| | and | | | | |
| | sulfoquinovosidase. | | | | |
| GH121 | β-L- | β-L- | Cereals (whole | Carrot, radish, | Carrot pectin, larch |
| | arabinobiosidase. | arabinopyranose | grains) and roots | tomato, barley, | arabinogalactan, |
| | | (branches in | | soybean, peach, | Gum arabic |
| | | arabinogalactan | | pear, banana | |
| | | proteins). | | | |
| GH13 | α-amylase, pullulanase, | Linear α-1,4- | Resistant-starch | Corn, dextrin | High amylose |
| | cyclomaltodextrin | glucan | from cereals | | starch, resistant |
| | glucanotransferase, | (amylose and | (whole grains), | | dextrin, |
| | cyclomaltodextrinase, | amylopectin) | legumes, | | polydextrose |
| | trehalose-6- | | vegetables and | | |
| | phosphate hydrolase, | | roots | | |
| | oligo-α-glucosidase, | | | | |
| | maltogenic amylase, | | | | |
| | neopullulanase, α- | | | | |
| | glucosidase, | | | | |
| | maltotetraose- | | | | |
| | forming α-amylase, | | | | |
| | isoamylase, | | | | |
| | glucodextranase, | | | | |
| | maltohexaose- | | | | |
| | forming α-amylase, | | | | |
| | maltotriose-forming | | | | |
| | α-amylase, branching | | | | |
| | enzyme, trehalose synthase, | | | | |
| | 4-α-glucanotransferase, | | | | |
| | maltopentaose- | | | | |
| | forming α-amylase, | | | | |
| | amylosucrase, sucrose | | | | |
| | phosphorylase, | | | | |
| | malto-oligosyltrehalose | | | | |
| | trehalohydrolase, | | | | |
| | isomaltulose | | | | |
| | synthase, malto- | | | | |
| | oligosyltrehalose | | | | |
| | synthase, amylo-α- | | | | |
| | 1,6-glucosidase, α- | | | | |
| | 1,4-glucan: | | | | |
| | phosphate α- | | | | |
| | maltosyltransferase, | | | | |
| | 6′-P-sucrose | | | | |
| | phosphorylase, and | | | | |
| | amino acid transporter | | | | |
| GH43_29 | β-xylosidase, α-L- | α-1,5(α- | Pectins from fruits, | Wheat, peas, | Wheat |
| | arabinofuranosidase, | 1,2)(α-1,3)- | vegetables, | sugarbeet, | arabinoxylan, corn |
| | and α-1,2-L- | arabinofuranose | legumes and roots; | apple, | arabinoxylan, |
| | arabinofuranosidase | (arabinan, | hemicellulose | citruses, rye, | psyllium husk |
| | | arabinoxylan), | from cereals | barley, oat, | arabinoxylan, pea |
| | | β-1,4-xylose | (whole grains) | rice, corn, | fiber, sugarbeet |
| | | (arabinoxylan) | | and sorghum | pectin, apple |
| | | | | (whole grains) | pectin, citrus pectin |
| GH13_4 | Amylosucrase and | α-1,4-glucan | Resistant-starch | Corn, dextrin | High amylose |
| | sucrose hydrolase. | (amylose and | from cereals | | starch, resistant |
| | | amylopectin) | (whole grains), | | dextrin |
| | | | legumes, | | |
| | | | vegetables and | | |
| | | | roots | | |
| GH146 | β-L- | β-L- | Pectins from fruits, | Carrot, radish, | Carrot pectin, larch |
| | arabinofuranosidase | arabinofuranose | vegetables, | tomato, barley, | arabinogalactan, |
| | | (branches in | legumes and roots; | soybean, peach, | Gum arabic |
| | | arabinogalactan | hemicellulose | pear, banana | |
| | | proteins and | from cereals | | |
| | | from pectin | (whole grains) | | |
| | | side-chains) | | | |
| GH29 | α-L-fucosidase and | Terminal α-L- | Human milk | HMOs, brown | Fucoidan, |
| | α-1,3/1,4-L- | fucosyl | oligosaccharides | algae PS | fucosylated HMOs |
| | fucosidase | linkages | | (Fucoidan) | |
| GH120 | β-xylosidase | β-1,4-xylose | Cereals (whole | Wheat, rye, | Wheat |
| | | (arabinoxylan); | grains) or sea | barley, oat, | arabinoxylan, corn |
| | | β-1,3(β-1,4)- | weed dietary fiber | rice (non | arabinoxylan, |
| | | xylose (xylan) | | refined) | psyllium husk |
| | | | | | arabinoxylan |
| GH43_11 | β-1,3-xylosidase, β- | α-1,5(α- | Pectins from fruits, | Wheat, peas, | Wheat |
| | xylosidase, and α-L- | 1,2)(α-1,3)- | vegetables, | sugarbeet, apple, | arabinoxylan, corn |
| | arabinofuranosidase. | arabinofuranose | legumes and roots; | citruses, rye, | arabinoxylan, |
| | | (arabinan, | hemicellulose | barley, oat, | psyllium husk |
| | | arabinoxylan), | from cereals | rice (whole | arabinoxylan, pea |
| | | β-1,4-xylose | (whole grains) | grain) | fiber, sugarbeet |
| | | (arabinoxylan) | | | pectin, apple |
| | | | | | pectin, citrus pectin |
| GH43_12 | β-xylosidase and α-L- | α-1,5(α- | Pectins from fruits, | Wheat, peas, | Wheat |
| | arabinofuranosidase. | 1,2)(α-1,3)- | vegetables, | sugarbeet, apple, | arabinoxylan, corn |
| | | arabinofuranose | legumes and roots; | citruses, rye, | arabinoxylan, |
| | | (arabinan, | hemicellulose | barley, oat, | psyllium husk |
| | | arabinoxylan), | from cereals | rice (whole grain) | arabinoxylan, pea |
| | | β-1,4-xylose | (whole grains) | | fiber, sugarbeet |
| | | (arabinoxylan) | | | pectin, apple |
| | | | | | pectin, citrus pectin |
| GH4 | Different functions: | Linear α-1,4- | Resistant-starch | Corn | High amylose |
| | maltose-6-phosphate | glucan | from cereals | | starch, resistant |
| | glucosidase, α- | (amylose and | (whole grains), | | dextrin |
| | glucosidase, α- | amylopectin) | legumes, | | |
| | galactosidase, 6- | | vegetables and | | |
| | phospho-β- | | roots | | |
| | glucosidase, α- | | | | |
| | glucuronidase, α- | | | | |
| | galacturonase, and | | | | |
| | palatinase. | | | | |
| GH43_24 | Exo-β-1,3- | β-1,3- | Fruit and vegetable | Carrot, radish, | Apple pectin, |
| | galactanase. | galactose | pectins and cereals | tomato, barley, | carrot pectin, larch |
| | | (galactan, | (whole grain) | soybean, peach, | arabinogalactan, |
| | | arabinogalactan) | | pear, banana, | Gum arabic |
| | | | | apple | |
| GH43_22 | β-xylosidase. | β-1,4-xylose | Cereals (whole | Wheat, rye, | Wheat |
| | | (arabinoxylan); | grains) or sea | barley, oat, | arabinoxylan, corn |
| | | β-1,3(β-1,4)- | weed dietary fiber | rice, corn, | arabinoxylan, |
| | | xylose (xylan) | | and sorghum | psyllium husk |
| | | | | (non refined) | arabinoxylan |
| GH30_5 | Endo-β-1,6- | β-1,6- | Fruit and vegetable | Apple, | Apple pectin |
| | galactanase. | galactose | pectins and cereals | banana, | |
| | | (galactan) | (whole grain) | radish | |
| GH85 | Endo-β-N- | GlcNAc-β- | N-linked | N-glycosylated | Hyaluronic acid, |
| | acetylglucosaminidase. | 1,4-GlcNac | oligosaccharides | proteins (mainly | chondroitin sulfate, |
| | | (N-linked | from fruits, nuts, | animal source) | heparin/heparan |
| | | glycans) | vegetables, cereals and | | |
| | | | animal sources. | | |
| GH127 | β-L- | β-L- | Pectins from fruits, | Carrot, radish, | Carrot pectin, |
| | arabinofuranosidase | arabinofuranose | vegetables, | tomato, barley, | larch |
| | and 3-C-carboxy-5- | (branches in | legumes and roots; | soybean, peach, | arabinogalactan, |
| | deoxy-L-xylose | arabinogalactan | hemicellulose | pear, banana | Gum arabic |
| | (aceric acid) | proteins and | from cereals | | |
| | hydrolase. | from pectin | (whole grains) | | |
| | | side-chains) | | | |
| GH43_10 | β-xylosidase and α-L- | α-1,5(α- | Pectins from fruits, | Wheat, peas, | Wheat |
| | arabinofuranosidase. | 1,2)(α-1,3)- | vegetables, | sugarbeet, | arabinoxylan, corn |
| | | arabinofuranose | legumes and roots; | apple, | arabinoxylan, |
| | | (arabinan, | hemicellulose | citruses, rye, | psyllium husk |
| | | arabinoxylan), | from cereals | barley, oat, | arabinoxylan, pea |
| | | β-1,4-xylose | (whole grains) | rice (whole | fiber, sugarbeet |
| | | (arabinoxylan) | | grain) | pectin, apple |
| | | | | | pectin, citrus pectin |
| GH101 | Endo-α-N- | GalNAc; | Mucin | | |
| | acetylgalactosaminidase. | Galβ1-3GalNAc | | | |
| | | (host-glycans) | | | |
| GH27 | α-galactosidase, | α-1,6- | Gum (guar, locust | Soybean hulls, | Galactooligo- |
| | isomalto- | galactopyranose | bean, fenugreek | clusterbean | saccharides |
| | dextranase, β-L- | (galactomannans); | alfalfa); galacto- | ((Cyamopsis | (GOS), larch |
| | arabinopyranosidase, | β-L- | oligosaccharides | tetragonoloba | arabinogalactan, |
| | and galactan: galactan | arabinopyranose | (GOS); pectins | L. Taub) | Gum arabic |
| | galactosyltransferase. | (branches in | from fruits, | | |
| | | arabinogalactan | vegetables, | | |
| | | proteins and | legumes and roots; | | |
| | | from pectin | hemicellulose | | |
| | | side-chains) | from cereals | | |
| | | | (whole grains) | | |
| GH43_16 | β-xylosidase and α-L- | α-1,5(α- | Pectins from fruits, | Wheat, peas, | Wheat |
| | arabinofuranosidase | 1,2)(α-1,3)- | vegetables, | sugarbeet, | arabinoxylan, corn |
| | | arabinofuranose | legumes and roots; | apple, | arabinoxylan, |
| | | (arabinan, | hemicellulose | citruses, rye, | psyllium husk |
| | | arabinoxylan), | from cereals | barley, oat, | arabinoxylan, pea |
| | | β-1,4-xylose | (whole grains) | rice (whole | fiber, sugarbeet |
| | | (arabinoxylan) | | grain) | pectin, apple |
| | | | | | pectin, citrus pectin |
| GH43_27 | β-xylosidase and α-L- | α-1,5(α- | Pectins from fruits, | Wheat, peas, | Wheat |
| | arabinofuranosidase | 1,2)(α-1,3)- | vegetables, | sugarbeet, | arabinoxylan, corn |
| | | arabinofuranose | legumes and roots; | apple, | arabinoxylan, |
| | | (arabinan, | hemicellulose | citruses, rye, | psyllium husk |
| | | arabinoxylan), | from cereals | barley, oat, | arabinoxylan, pea |
| | | β-1,4-xylose | (whole grains) | rice (whole | fiber, sugarbeet |
| | | (arabinoxylan) | | grain) | pectin, apple |
| | | | | | pectin, citrus pectin |
| GH95 | α-L-fucosidase, α- | Terminal α-L- | Human milk | HMOs, | Fucoidan, |
| | 1,2-L-fucosidase, | fucosyl | oligosaccharides | brown algae | fucosylated HMOs |
| | and α-L- | linkages; α-1,6- | | PS (Fucoidan) | |
| | galactosidase. | galactopyranose | | (galactomannan | |
| | | (galactomannans) | | in guar gum) | |
| GH33 | Sialidase or | Terminal α- | Human milk | HMOs, | Sialylated HMOs |
| | neuraminidase, | 2,3(α-2,6)- | oligosaccharides | animal source | |
| | trans-sialidase, | sialyl linkages | | ingredients | |
| | anhydrosialidase, | | | (glycoproteins | |
| | Kdo hydrolase, and | | | and | |
| | 2-keto-3- | | | glycolipids) | |
| | deoxynononic acid | | | | |
| | hydrolase/KDNase. | | | | |
| GH18 | Chitinase, | β-1,4-N- | Crustacean shells | Chitin, | Chitosan |
| | lysozyme, endo-β-N- | acetyl-D- | and fungi; N- | chitosan in | |
| | acetylglucosaminidase, | glucosamine | linked | mushroom | |
| | peptidoglycan | (chitin); | oligosaccharides | extracts | |
| | hydrolase with | GlcNAc-β- | from fruits, nuts, | (Namex) | |
| | endo-β-N- | 1,4-GlcNac | vegetables, and | | |
| | acetylglucosaminidase | (N-linked | cereals | | |
| | specificity, Nod | glycans) | | | |
| | factor hydrolase, | | | | |
| | xylanase inhibitor, | | | | |
| | concanavalin B, | | | | |
| | and narbonin. | | | | |
| GH43_34 | α-L- | α-1,5(α- | Pectins from fruits, | Carrot, radish, | Carrot pectin, larch |
| | arabinofuranosidase | 1,2)(α-1,3)- | vegetables, | tomato, barley, | arabinogalactan, |
| | and β-D- | arabinofuranose | legumes and roots; | soybean, peach, | Gum arabic, pea |
| | galactofuranosidase | (arabinan, | hemicellulose | pear, banana | fiber, sugarbeet |
| | | arabinoxylan); | from cereals | | pectin |
| | | β-1,4(β-1,3)- | (whole grains) | | |
| | | galactose | | | |
| | | (arabinogalactan | | | |
| | | and galactan) | | | |

Table 3 provides details of CAZymes that were present in all Bifidobacterium longum strains analysed (i.e., Bifidobacterium longum transitional, Bifidobacterium longum subsp. suis/suillum, Bifidobacterium longum subsp. longum and Bifidobacterium longum subsp. infantis). Table 3 also provides a summary of the glycan substrate metabolized by each CAZyme and illustrative dietary fiber sources/ingredients.

TABLE 3

| CAZyme | Function | Glycan substrate | Dietary fiber source |
|---|---|---|---|
| GH3 | Different functions: β-glucosidase, xylan | β-1,3(β-1,4)- | Cereals (whole grains) |
| | 1,4-β-xylosidase, β-glucosylceramidase, | glucose (β-glucans) | |
| | β-N-acetylhexosaminidase, α-L- | | |
| | arabinofuranosidase, glucan 1,3-β- | | |
| | glucosidase, glucan 1,4-β-glucosidase, | | |
| | isoprimeverose-producing | | |
| | oligoxyloglucan hydrolase, coniferin β- | | |
| | glucosidase, exo-1,3-1,4-glucanase, and | | |
| | β-N-acetylglucosaminide | | |
| | phosphorylases. | | |
| GH23 | Lysozyme type G, peptidoglycan lyase | (GlcNAc-MurNAc)n | No dietary source |
| | (also known in the literature as | (bacterial | |
| | peptidoglycan lytic transglycosylase), | peptidoglycan) | |
| | and chitinase. | | |
| GH42 | β-galactosidase and α-L- | α-arabinopyranose; | Pectins from fruits, |
| | arabinopyranosidase. | β-1,4(β-1,3)- | vegetables, legumes and roots; |
| | | galactose | hemicellulose |
| | | (arabinogalactan | from cereals (whole grains) |
| | | and galactan) | |
| GH51 | Endoglucanase, endo-β-1,4-xylanase, β- | β-1,3(β-1,4)- | Cereals (whole grains) |
| | xylosidase, and α-L-arabinofuranosidase. | glucose (β-glucans) | |
| GH38 | α-mannosidase, mannosyl- | α-1,6-mannose | Manooligosaccharides (MOS) |
| | oligosaccharide α-1,2-mannosidase, | (mannose | |
| | mannosyl-oligosaccharide α-1,3-1,6- | oligosaccharides; | |
| | mannosidase, α-2-O-mannosylglycerate | MOS) | |
| | hydrolase, and mannosyl- | | |
| | oligosaccharide α-1,3-mannosidase. | | |
| GH13_11 | Isoamylase. | α-1,6-glucan | Resistant-starch from cereals |
| | | (amylopectin) | (whole grains), legumes, |
| | | | vegetables and roots |
| GH77 | Amylomaltase or 4-α- | α-1,4-glucan | Resistant-starch from cereals |
| | glucanotransferase. | (starch) | (whole grains), legumes, |
| | | | vegetables and roots |
| GH2 | Different functions: β-galactosidase, β- | β-1,3(β-1,4)(β-1,6)- | Galactooligosaccharides |
| | mannosidase, β-glucuronidase, α-L- | galactose, β-1,3- | (GOS); pectins from fruits, |
| | arabinofuranosidase, | galactobiose/triose | vegetables, legumes and roots; |
| | mannosylglycoprotein endo-β- | (GOS, galactans | human milk oligosaccharides |
| | mannosidase, exo-β-glucosaminidase, α- | and | |
| | L-arabinopyranosidase, and β- | arabinogalactans) | |
| | galacturonidase. | | |
| GH20 | β-hexosaminidase, lacto-N-biosidase, β- | Galβ1- | Human milk oligosaccharides |
| | 1,6-N-acetylglucosaminidase, and β-6- | 3GlcNAcβ1- | |
| | SO3-N-acetylglucosaminidase. | 3Galβ1-4Glc | |
| | | (lacto-N-tetraose) | |
| GH32 | Invertase, endo-inulinase, β-2,6-fructan | β-1,2(β-2,6)-D- | Chicory root, agave, onion, |
| | 6-levanbiohydrolase, endo-levanase, | fructofuranose | jerusalem artichoke, oat |
| | exo-inulinase, fructan β-(2,1)- | (furctans like | |
| | fructosidase/1-exohydrolase, fructan β- | inulin, raffinose, | |
| | (2,6)-fructosidase/6-exohydrolase, | levan, graminan). | |
| | sucrose: sucrose 1-fructosyltransferase, | | |
| | fructan: fructan 1-fructosyltransferase, | | |
| | sucrose: fructan 6-fructosyltransferase, | | |
| | fructan: fructan 6G-fructosyltransferase, | | |
| | levan fructosyltransferase, [retaining] | | |
| | sucrose: sucrose 6-fructosyltransferase | | |
| | (6-SST), and cycloinulo-oligosaccharide | | |
| | fructanotransferase | | |
| GH13_30 | α-glucosidase. | α-1,4-glucan | Resistant-starch from cereals |
| | | (amylose and | (whole grains), legumes, |
| | | amylopectin) | vegetables and roots |
| GH13_31 | Oligo-α-1,6-glucosidase and | α-1,6-glucan | Resistant-starch from cereals |
| | glucodextranase. | (amylopectin) | (whole grains), legumes, |
| | | | vegetables and roots |
| GH25 | Lysozyme | (GlcNAc- | No dietary source |
| | | MurNAc)n | |
| | | (bacterial | |
| | | peptidoglycan) | |
| GH36 | α-galactosidase, α-N- | α-1,6- | Gum (guar, locust bean, |
| | acetylgalactosaminidase, stachyose | galactopyranose | fenugreek alfalfa); |
| | synthase, and raffinose synthase. | (galactomannans; | galactooligosaccharides |
| | | raffinose and | (Alpha-GOS); vegetables and |
| | | stachyose | legumes |
| | | oligosaccharides) | |
| GH112 | Lacto-N-biose phosphorylase or galacto- | Galβ1, 3GalNAc, | Human milk oligosaccharides |
| | N-biose phosphorylase, D-galactosyl-β- | GNB (galacto-N- | |
| | 1,4-L-rhamnose phosphorylase, and | biose); Galβ1, | |
| | galacto-N-biose/lacto-N-biose | 3GlcNAc, LNB | |
| | phosphorylase. | (lacto-N-biose I) | |
| GH125 | Exo-α-1,6-mannosidase. | α-1,6-mannose | MOS |
| | | (mannose | |
| | | oligosaccharides; | |
| | | MOS) | |
| GH129 | α-N-acetylgalactosaminidase. | GalNAc; Galβ1- | Mucins |
| | | 3GalNAc (host- | |
| | | glycans) | |
| GH13_18 | Sucrose phosphorylase and 6′-P-sucrose | Sucrose | Sucrose and raffinose |
| | phosphorylase. | | |
| GH13_20 | Cyclic a-1,6-maltosyl-maltose hydrolase; | α-1,4(α-1,6)-glucan | Resistant-starch from cereals |
| | α-glycosidase hydrolyzing pullulan, | (amylose and | (whole grains), legumes, |
| | starch and g-cyclodextrin; α-amylase; | amylopectin) | vegetables and roots |
| | maltogenic α-amylase; neopullulanase; | | |
| | pullulanase; cyclomaltodextrinase | | |
| GH13_3 | α-1,4-glucan and phosphate α- | α-1,4-glucan | Resistant-starch from cereals |
| | maltosyltransferase. | (starch) | (whole grains), legumes, |
| | | | vegetables and roots |
| GH13_9 | α-1,4-glucan branching enzyme. | α-1,4-glucan | Resistant-starch from cereals |
| | | (amylose and | (whole grains), legumes, |
| | | amylopectin) | vegetables and roots |
| GH5_18 | β-mannosidase. | Manβ1-4GlcNAc | Host glycans, glycoproteins. |
| | | disaccharide (N- | |
| | | glycans) | |
| GH53 | Endo-β-1,4-galactanase. | β-1,4-galactose | Different sources of fruit and |
| | | (galactan, | vegetable pectins and cereals |
| | | arabinogalactan) | (whole grain) |
| GH5_44 | β-glycosidase. | β-glucose (β- | Cereals (whole grains) |
| | | glucans) | |

Table 4 provides details of the CAZymes that are not encoded by Bifidobacterium longum transitional strains but are encoded by one or more of Bifidobacterium longum subsp. suis/suillum, Bifidobacterium longum subsp. longum and Bifidobacterium longum subsp. infantis.

TABLE 4

| CAZyme | Function | Glycan substrate |
|---|---|---|
| GH43_4 | [Inverting] endo-α-1,5-L-arabinanase. | α-1,5-arabinofuranose |
| | | (arabinan) |
| GH43_26 | Exo-α-1,5-L-arabinofuranosidase and α-L- | α-1,5-arabinofuranose |
| | arabinofuranosidase. | (arabinan) |
| GH16 | Xyloglucan: xyloglucosyltransferase, keratan-sulfate endo- | β-1,4-galactose (galactan, |
| | 1,4-β-galactosidase, endo-1,3-β-glucanase, endo-1,3(4)-β- | arabinogalactan); β-glucose (β- |
| | glucanase, licheninase, β-agarase, k-carrageenase, | glucans and xyloglucans) |
| | xyloglucanase, endo-β-1,3-galactanase, β-porphyranase, | |
| | hyaluronidase, endo-β-1,4-galactosidase, chitin β-1,6- | |
| | glucanosyltransferase, and endo-β-1,4-galactosidase. | |
| GH136 | Lacto-N-biosidase. | lacto-N-tetraose, lacto-N- |
| | | hexaose, lacto-N-fucopentaose |
| | | I, and sialyllacto-N-tetraose |
| GH1 | Different functions: β-glucosidase, β-galactosidase, β- | α-1,5-arabinofuranose (arabino |
| | mannosidase, β-glucuronidase, β-xylosidase, β-D-fucosidase, | oligosaccharides; AOS), β-D- |
| | phlorizin hydrolase, exo-β-1,4-glucanase, 6-phospho-β- | xylose (xylose oligosaccharides; |
| | galactosidase, 6-phospho-β-glucosidase, strictosidine β- | XOS) |
| | glucosidase, lactase, amygdalin β-glucosidase, prunasin β- | |
| | glucosidase, vicianin hydrolase, raucaffricine β-glucosidase, | |
| | thioglucosidase, β-primeverosidase, isoflavonoid 7-O-β- | |
| | apiosyl-β-glucosidase, ABA-specific β-glucosidase, | |
| | DIMBOA β-glucosidase, β-glycosidase, and hydroxyisourate | |
| | hydrolase. | |
| GH109 | α-N-acetylgalactosaminidase. | GalNAc; Galβ1-3GalNAc |
| | | (host-glycans) |
| GH151 | α-L-fucosidase. | α-L-fucosyl linkages |
| PL27 | L-rhamnose-α-1,4-D-glucuronate lyase | Terminal α-l-rhamnopyranosyl |

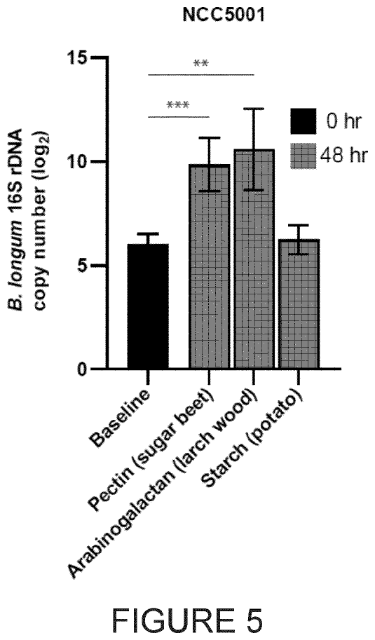

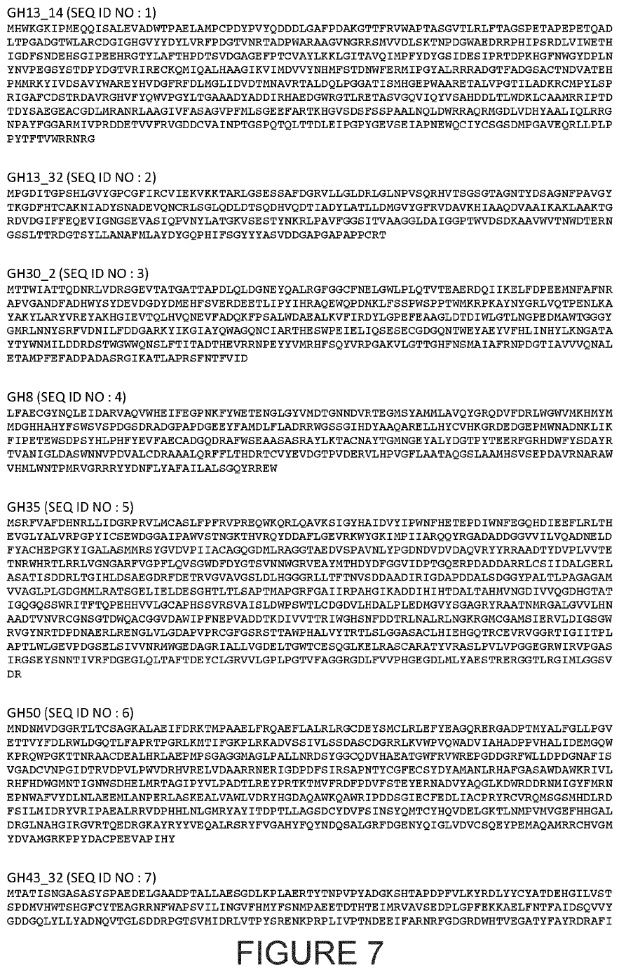

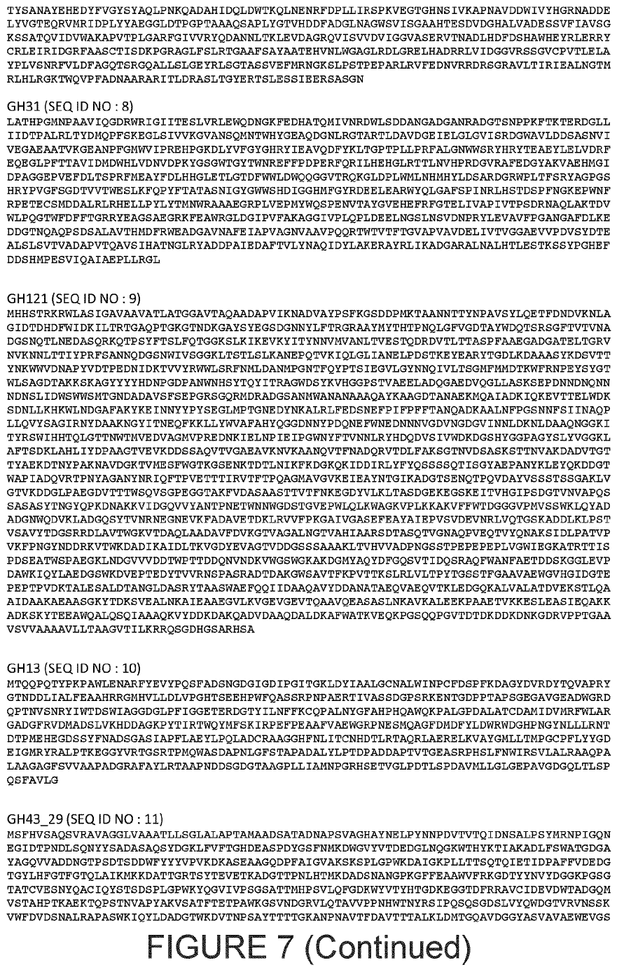

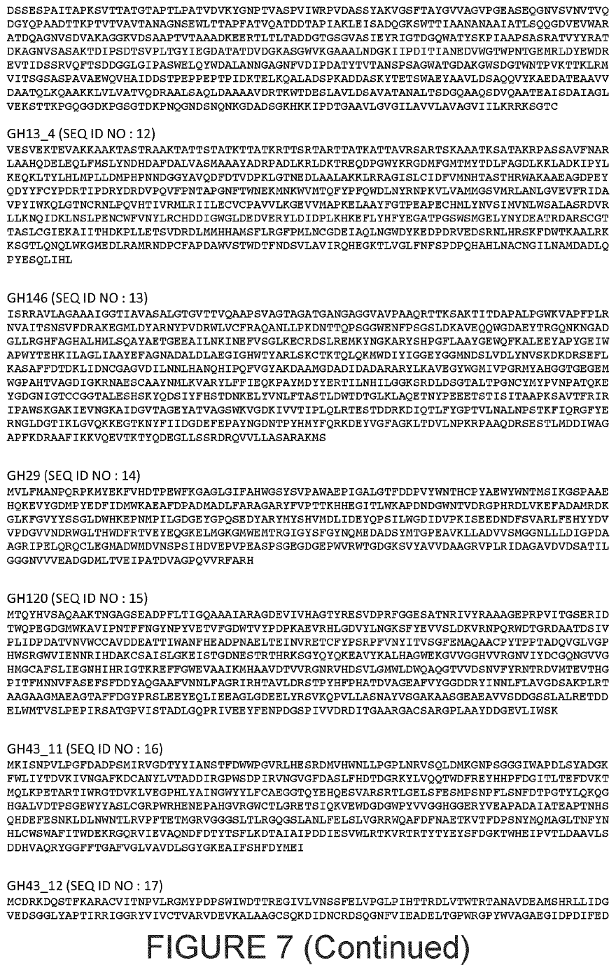

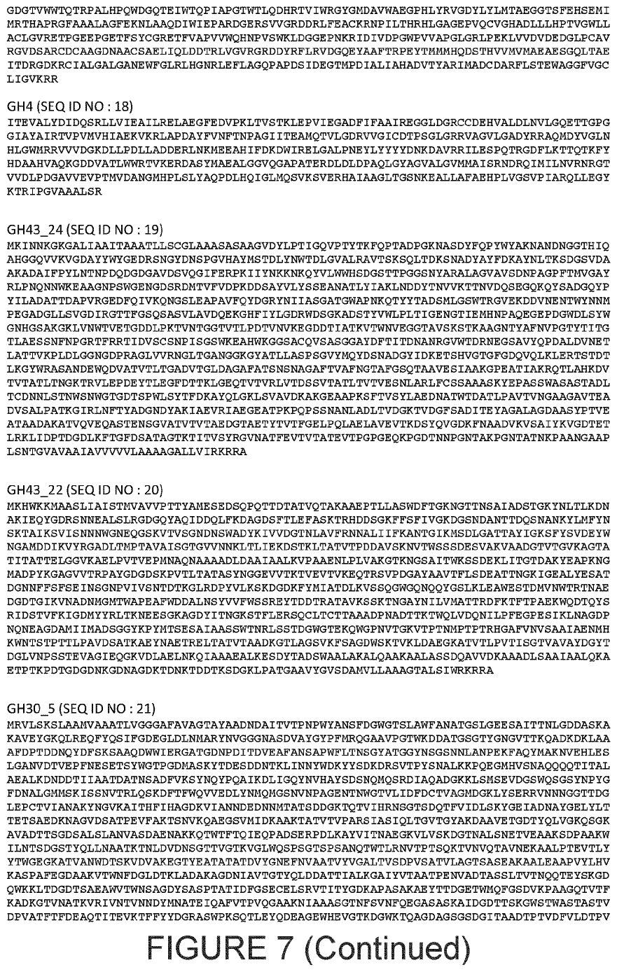

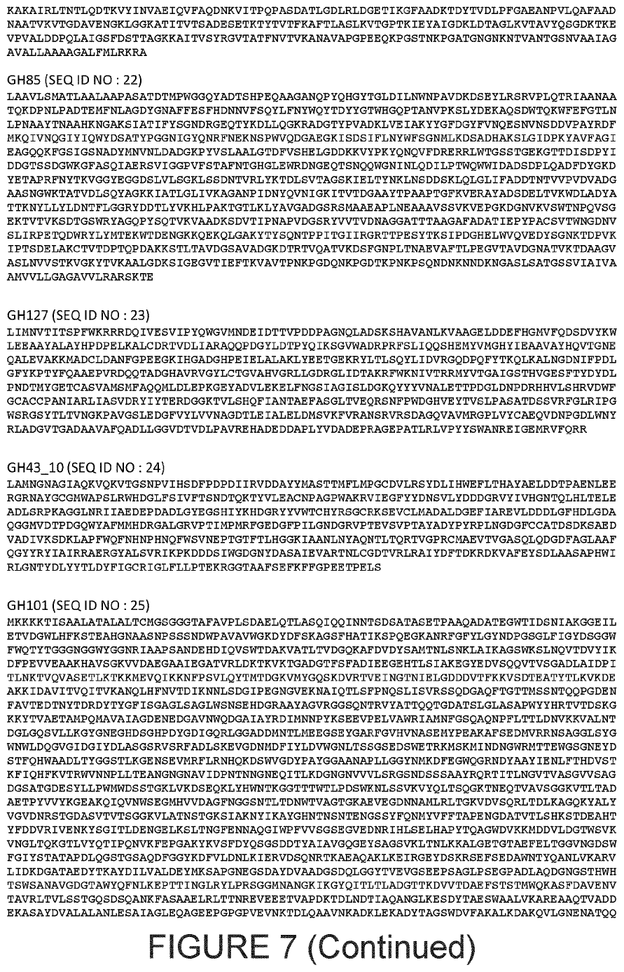

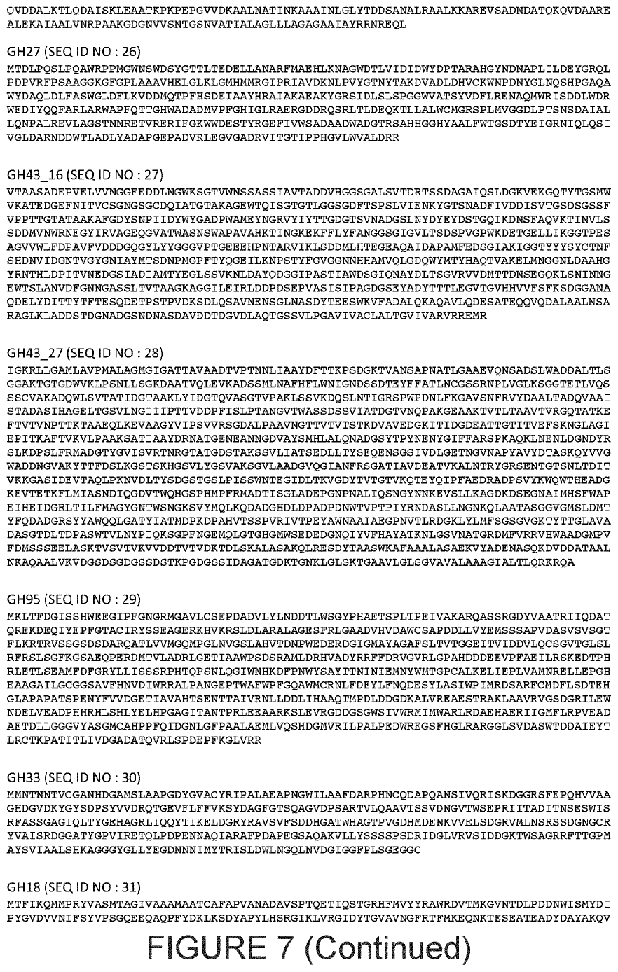

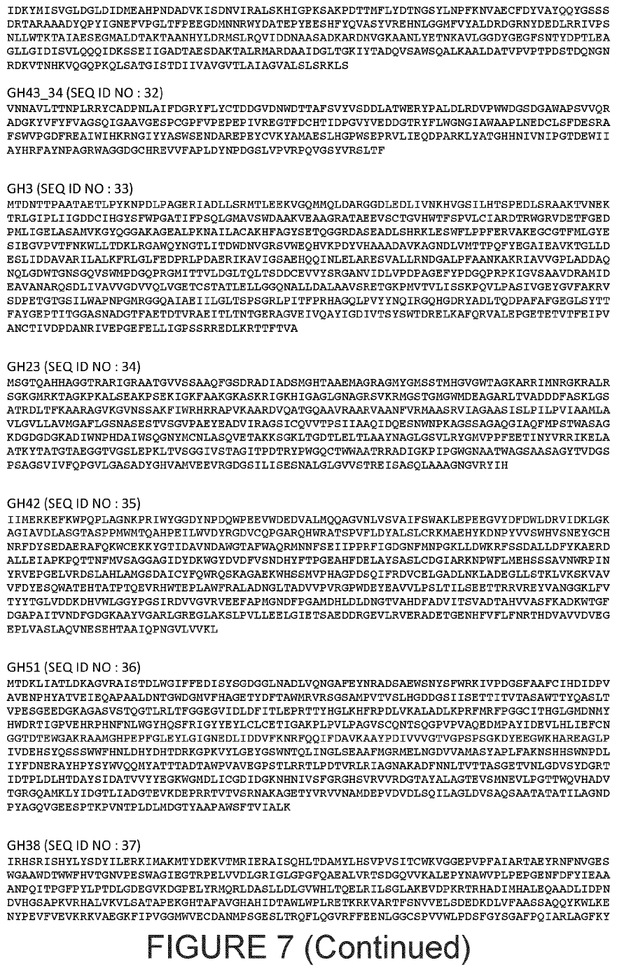

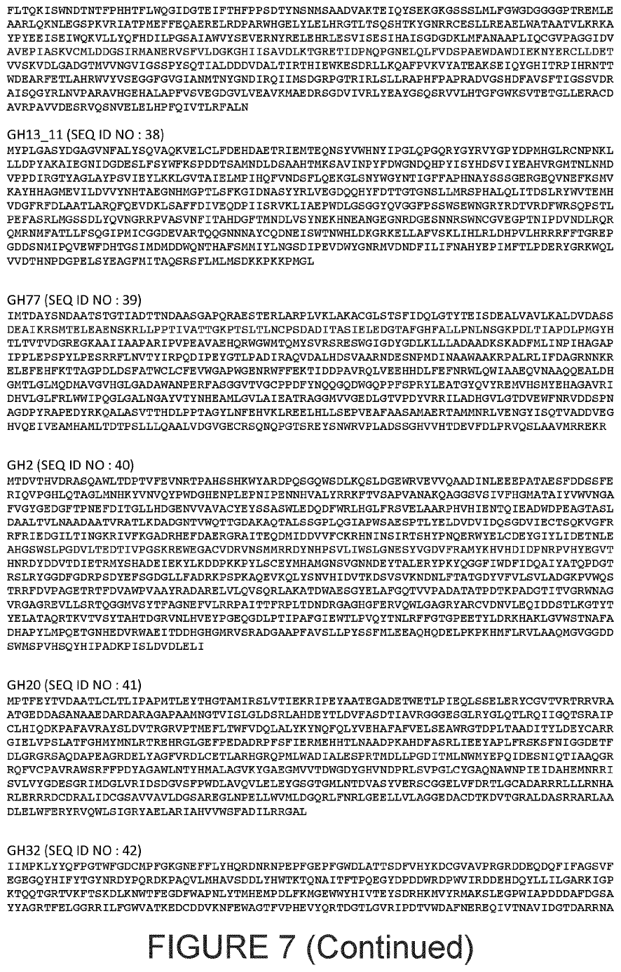

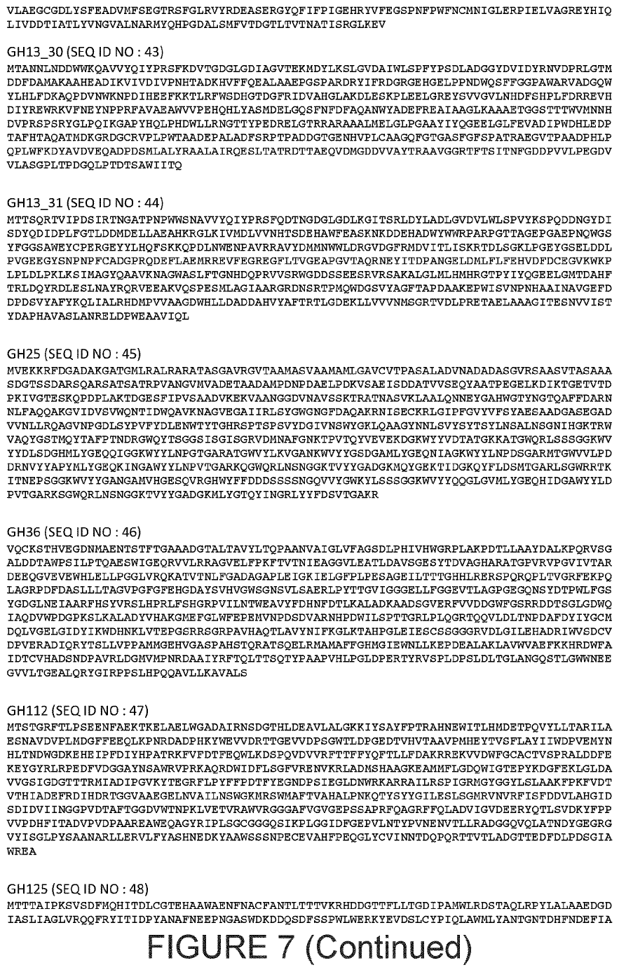

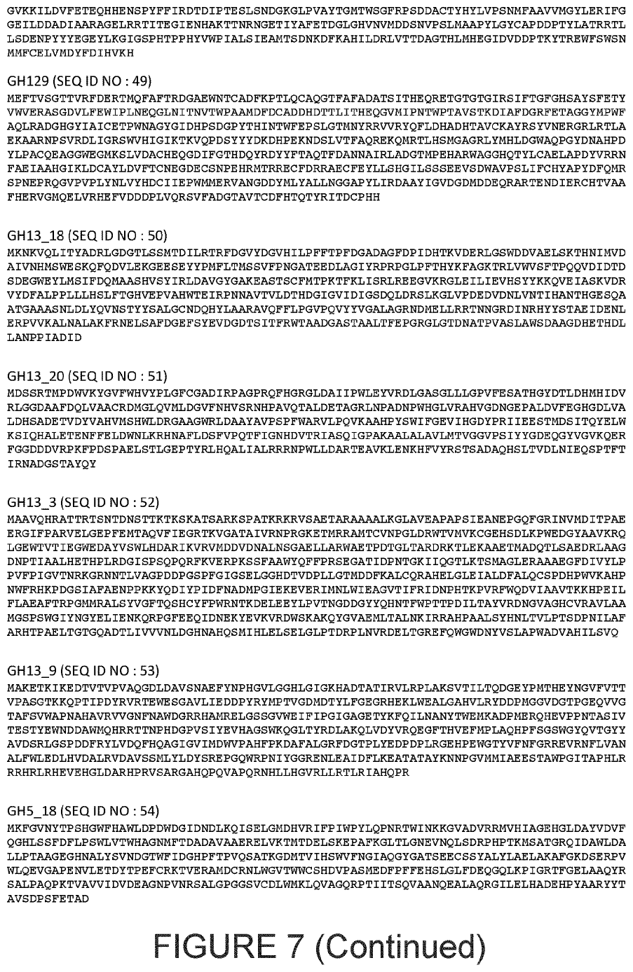

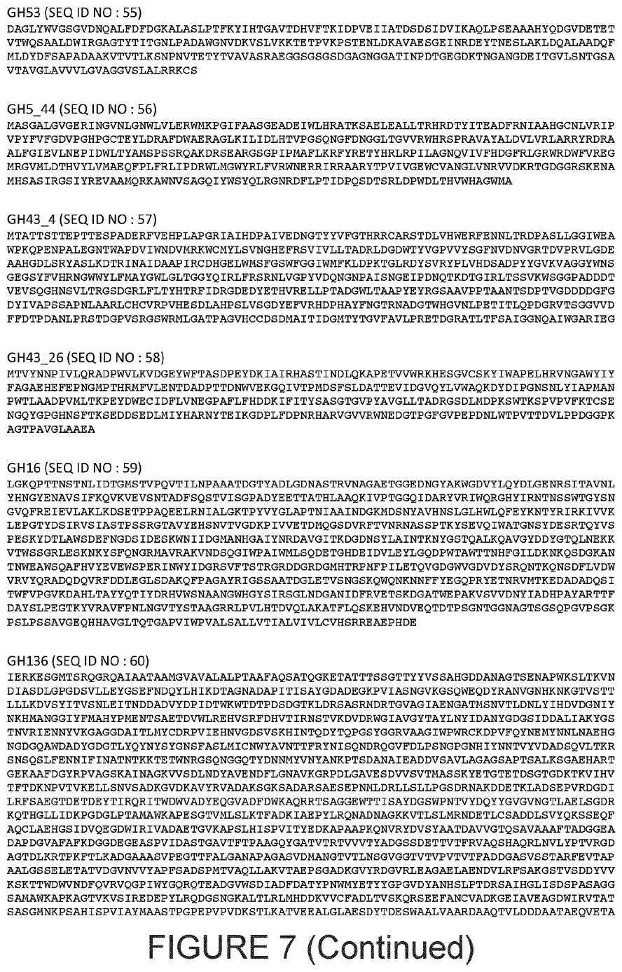

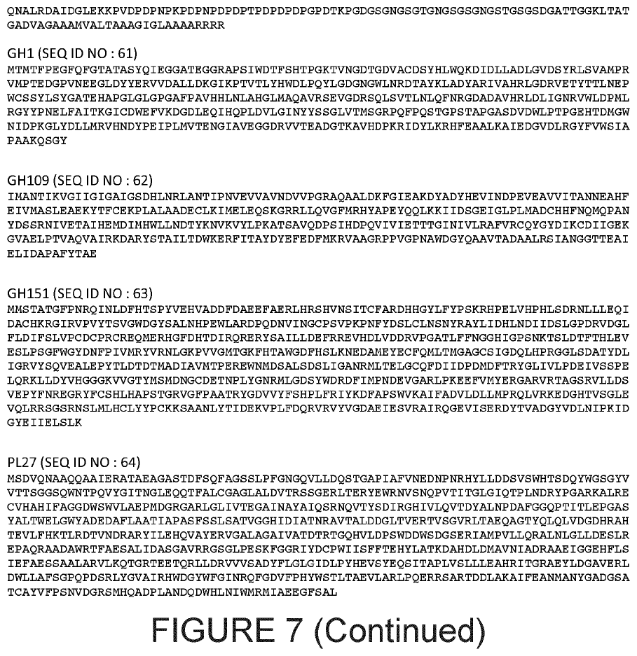

Representative sequences for the CAZymes listed in Tables 1~4 are shown in FIG. 7. Suitably, the CAZyme referred to in any of Tables 1~4 may comprise or consist of the corresponding sequence shown in FIG. 7. Suitably, the CAZyme may comprise or consist of a variant of the corresponding sequence shown in FIG. 7, which variant retains at least one of the functions of the corresponding CAZyme as recited in Table 1-4. Suitably, the variant may provide each of the functional activities of the corresponding CAZyme as recited in Table 1-4. Suitably, the variant may comprise or consist of an amino acid sequence which has at least 70% sequence identity to the sequence listed in FIG. 7, and retains at least one of the functional activities, preferably each of the functional activities, of the corresponding CAZyme as recited in Table 1-4. Suitably, the variant may comprise or consist of an amino acid sequence, which has at least 70%, at least 75%, at least 80%, at least 85%, at least 90%, at least 95%, at least 96%, at least 97%, at least 98%, or at least 99% sequence identity to the corresponding sequence listed in FIG. 5. The variant retains at least one of the functional activities, preferably each of the functional activities, of the corresponding CAZyme as recited in Table 1-4.

Suitably, the prebiotic for use in the present invention may comprise a glycan substrate selected from the groups recited in any of Tables 1 to 3.

Suitably, the prebiotic for use in the present invention may comprise a combination of glycan substrates selected from the groups recited in any of Tables 1 to 3.

The combination of glycan substrates may comprise at least 2, at least 4, at least 10, at least 20, at least 30, at least 40 or at least 50 of the glycan substrates selected from the groups recited in Tables 1 to 3. The combination may comprise each of the glycan substrates recited in Tables 1 to 3.

Suitably, the prebiotic may comprise one or more glycan substrates selected from the group recited in Table 1 or Table 2.

The prebiotic may comprise at least 2, at least 4, at least 10, at least 20, or at least 30 of the glycan substrates recited in Tables 1 and 2. The prebiotic may comprise each of the glycan substrates recited in Tables 1 and 2.

Suitably, the glycan substrate may comprise or consist of pectin, arabinogalactan and/or starch.

Suitably, the glycan substrate may comprise or consist of pectin.

Suitably, the glycan substrate may comprise or consist of arabinogalactan.

Suitably, the glycan substrate may comprise or consist of starch.

Suitably, the glycan substrate is provided in the form of a dietary fiber. For example, the dietary fiber may be a prebiotic fiber.

Suitably, the glycan substrate may be comprised in an ingredient, for example a dietary ingredient.

The ingredient containing one or several glycan substrates may be selected from the group consisting of purified polysaccharide or purified oligosaccharide, a dietary fiber ingredient, a semi-purified food ingredient, a raw food ingredient, a food additive, a HMO, a semi-purified or purified peptido-glycan.

The semi-purified food ingredient may be a fruit, vegetable or cereal extract.

The raw food ingredient may be a fruit, vegetable, cereal, algae or microalgae.

The food additive may be a guar gum or gum arabic.

Suitably, the peptide-glycan may be a GAG.

Suitably, the glycan substrate may be comprised in a purified fiber.

Illustrative ingredients and/or purified fibers comprising suitable glycan substrates are provided in Tables 1 to 3. In particular, dietary fibers and/or ingredients that comprise a given glycan substrate are identified in the same row as the glycan substrate.

The pectin may be comprised in fruit or vegetable pectin. Accordingly, suitable ingredients comprising pectin include, but are not limited to, fruits (e.g., apple, pear), vegetables, legumes (peas), and roots (e.g., sugar beet). Suitable purified fibers comprising arabinogalactan include peach pectin. Suitably, the pectin extracted from sugar beet contains arabinan, galactans and arabinogalactans and may be provided as an ingredient.

The arabinogalactan may be comprised in fruit or vegetable pectin. Illustrative suitable ingredients comprising arabinogalactan include, but are not limited to, fruits, vegetables, whole grain cereals and sea weed dietary fiber. Suitable purified fibers comprising arabinogalactan include peach pectin, larch wood arabinogalactan, and Arabic gum. Suitably, the arabinogalactan may be provided in larch wood arabinogalactan.

The starch may be comprised in resistant-starch from cereals (whole grains), legumes, vegetables (e.g., corn) and roots (e.g., potato). Illustrative suitable ingredients comprising starch include, but are not limited to, corn. Suitable purified fibers comprising starch include high amylose starch and resistant dextrin. Suitably, the starch may be provided in a potato, corn or other ingredient. Suitably, the starch may be comprised in a potato ingredient.

### Human Milk Oligosaccharide (HMO)

Suitably, the prebiotic comprises an HMO.

Suitably, the HMO is capable of being metabolized by the B longum transitional microorganism. Suitably, the HMO may be capable of promoting growth and/or survival of the B. longum transitional strain. HMOs capable of promoting growth and/or survival of the B. longum transitional strain may be determined by e.g. anaerobic culture of the B. longum transitional strain with the HMO to be tested. Growth and/or survival of the B. longum transitional strain may be determined by measuring bacteria cell number, cell density (e.g. measured by optical density) and/or the abundance of 16S rDNA—for example using PCR methods. An illustrative assay for measuring growth of a B. longum transitional strain in the presence of HMOs is provided in present Example 6. An HMO capable of promoting growth and/or survival of the B. longum transitional strain may increase the number of B. longum transitional bacteria in an anaerobic culture by at least 20%, at least 30%, at least 40%, at least 50%, at least 75% or at least 100% compared to the number of B. longum transitional bacteria in a control anaerobic culture which does not comprise the HMO. Suitably, HMO capable of promoting growth and/or survival of the B. longum transitional strain may increase the number of B. longum transitional bacteria in an anaerobic culture by a statistically significant amount (e.g. p-value <0.05 as determined by one-way ANOVA) compared to the number of B. longum transitional bacteria in a control anaerobic culture which does not comprise the HMO.

The HMO may be a fucosylated oligosaccharide (i.e. an oligosaccharide having a fucose residue; e.g. 2′ fucosyllactose (2-FL), 3-fucosyllactose (3-FL), difucosyllactose (DiFL), lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof), an N-acetylated oligosaccharide (e.g. LNT (lacto-N-tetraose), para-lacto-N-neohexaose (para-LNnH), LNnT (lacto-N-neotetraose), DSLNT (disialyllacto-N-tetraose), lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose and any combinations thereof) and/or a sialylated oligosaccharide (e.g. 3′-sialyllactose (3-SL), 6′-sialyllactose (6-SL), or Lst (sialyllacto-N-tetraose), Lst-a, Lst-b or Lst-c)).

The prebiotic may comprise at least one prebiotic oligosaccharide selected from the group consisting of: 2′-O-fucosyllactose (2′FL), 3′-O-fucosyllactose (3FL), lactodifucotetraose/difucosyllactose (DFL), 3′-O-sialyllactose (3-SL), 6′-O-sialyllactose (6-SL), lacto-N-tetraose (LNT) and lacto-N-neotetraose (LNnT); and any combination thereof. The prebiotic may comprise 34 wt % to 85 wt % of 2′-FL, 10 wt % to 40 wt % of LNT, 4 wt % to 14 wt % of DFL and 9 wt % to 31 wt % of 3-SL and 6-SL combined.

In some embodiments, the prebiotic comprises
26 wt % to 65 wt % of 2′-FL, preferably 32 wt % to 54 wt %;
10 wt % to 40 wt % of LNT, preferably 11 wt % to 20 wt %;
4 wt % to 14 wt % of DFL, preferably 4 wt % to 8 wt %;
9 wt % to 31 wt % of 3′-SL and 6′-SL combined, preferably 8 wt % to 22 wt %; and
12 wt % to 38 wt % of 3-FL, preferably 17 wt % to 31 wt %.

The prebiotic may comprise between 0.001 g/L to 12 g/L of 2′-FL, preferably between 0.002 g/L to 10 g/L of 2′-FL, more preferably between 0.005 g/L to 5 g/L of 2′-FL.

The prebiotic may comprise between 0.001 g/L to 5 g/L of DFL, preferably between 0.002 g/L to 4 g/L of DFL, more preferably between 4 g/L to 3 g/L of DFL.

The prebiotic may comprise between 0.01 g/L to 6 g/L of LNT, preferably between 0.025 g/L to 5 g/L of LNT, more preferably between 0.05 g/L to 1 g/L of LNT.

The prebiotic may comprise between 0.001 g/L to 2 g/L of 6′-SL, preferably between 0.002 g/L to 1.5 g/L of 6′-SL, more preferably between 0.005 g/L to 1 g/L of 6′-SL.

The prebiotic may comprise between 0.01 g/L to 2 g/L of 3′-SL, preferably between 0.025 g/L to 1.5 g/L of 3′-SL, more preferably between 0.05 g/L to 1 g/L of 3′-SL.

The prebiotic may comprise between 0.01 g/L to 7 g/L of 3-FL, preferably between 0.025 g/L to 6 g/L of 3-FL, more preferably between 0.05 g/L to 5 g/L of 3-FL.

Suitably, the 3′-O-fucosyllactose (3′FL) and lacto-N-tetraose (LNT) comprised in the prebiotic promote the growth of a Bifidobacterium longum transitional microorganism that preferentially utilizes 3-fucosyllactose (3-FL) over 2′-fucosyllactose (2′-FL).

Combination of a B. longum Transitional Microorganism and a Prebiotic

The invention further provides a combination of a B longum transitional microorganism and a prebiotic for use according to the present invention.

The B. longum transitional microorganism and prebiotic may be administered separately, simultaneously or sequentially.

Suitably, the B. longum transitional microorganism and prebiotic may be administered in a combined composition.

Suitably, a combination of a B. longum transitional microorganism and a prebiotic may be referred to as a “synbiotic”.

In aspects of the invention in which a combination of a B. longum transitional microorganism and a prebiotic (e.g. a glycan substrate) are used, each may be selected such that the B longum transitional microorganism is capable of metabolising the glycan substrate provided in the combination. Such a selection may be made, for example, by selecting a B. longum transitional microorganism that encodes a CAZyme from the same row of Tables 1-3 as the glycan substrate (or selecting an ingredient comprising said glycan substrate).

The combinations of the invention are not limited to requiring that the B. longum transitional microorganism is capable of metabolizing the glycan substrate provided in the combination. As such, any combinations of B. longum transitional microorganism(s) and glycan substrates disclosed herein are encompassed by the invention.

Suitably, the composition comprises one or more glycan substrates as described herein.

Suitably, the composition comprises B. longum transitional preferentially utilizing 3-fucosyllactose (3-FL) over 2′-fucosyllactose (2′-FL) mixed with 3′-O-fucosyllactose (3-FL) and lacto-N-tetraose (LNT). The composition may comprise between 10³ to 10¹² cfu of probiotic strain, more preferably between 10⁷ and 10¹² cfu such as between 10⁸ and 10¹⁰ cfu of probiotic strain per g of composition on a dry weight basis mixed with 3-FI in an amount between 0.01 g/L to 7 g/L of 3-FL, preferably between 0.025 g/L to 6 g/L of 3-FL, more preferably between 0.05 g/L to 5 g/L of 3-FL and with LNT in an amount between 0.01 g/L to 6 g/L of LNT, preferably between 0.025 g/L to 5 g/L of LNT, more preferably between 0.05 g/L to 1 g/L of LNT.

### Compositions

The B. longum transitional microorganism, prebiotic or synbiotic for use in the present invention may be provided in the form of a composition.

The composition may suitably be administered to an individual, for example an infant or a young child, in any suitable form such as a nutritional composition in a dosage unit (for example a tablet, a capsule, a sachet of powder, etc). the composition may be in powder, semi-liquid or liquid form. The composition may be added to a nutritional composition, an infant formula, a food composition, a supplement for infant or young child, a baby food, a follow-up formula, a growing-up milk, an infant cereal or a fortifier. In some embodiments, the composition of the present invention is an infant formula, a baby food, an infant cereal, a growing-up milk, a supplement or fortifier that may be intended for infants or young child.

By way of example, the composition may comprise further components which may be beneficial in reducing the risk of developing an allergy and/or allergic reaction. In addition, or alternatively, the composition may comprise further components may be beneficial during the weaning period.

For example, the composition may comprise a further probiotic-such as a probiotic with known effects on reducing the risk of developing an allergy and/or allergic reaction—(e.g. B. lactis, L. rhamnosus, B. longum subsp. infantis), formula (e.g. partially hydrolysed formulae, extensively hydrolysed formulae, amino acid-based formulae, or intact formulae), baby food (with or without milk fat), milk fat, cereals, docosahexaenoic acid (DHA), eicosapentaenoic acid (EPA), butyrate and/or gamma-linolenic acid (GLA).

The B longum transitional microorganism can be included in the composition in an amount from about 10³ to 10¹² cfu of probiotic strain, more preferably between 10⁷ and 10¹² cfu such as between 10⁸ and 10¹⁰ cfu of probiotic strain per g of composition on a dry weight basis. In one embodiment, the B. longum transitional microorganism is viable. In another embodiment the B. longum transitional microorganism is non-replicating or inactivated. There may be both viable and inactivated Bifidobacterium longum transitional microorganisms in some other embodiments.

Suitably, the composition comprises one or more glycan substrates as described herein.

In some embodiments, the composition comprises at least one prebiotic oligosaccharide selected in the group consisting of 2′-O-fucosyllactose (2FL), 3′-O-fucosyllactose (3FL), lactodifucotetraose/difucosyllactose (DFL), 3′-O-sialyllactose (3′-SL), 6′-O-sialyllactose (6′-SL) and lacto-N-tetraose (LNT) and any combination thereof.

In some embodiments, the composition comprises
26 wt % to 65 wt % of 2′-FL, preferably 32 wt % to 54 wt %;
10 wt % to 40 wt % of LNT, preferably 11 wt % to 20 wt %;
4 wt % to 14 wt % of DFL, preferably 4 wt % to 8 wt %;
9 wt % to 31 wt % of 3′-SL and 6′-SL combined, preferably 8 wt % to 22 wt %; and
12 wt % to 38 wt % of 3-FL, preferably 17 wt % to 31 wt %.

The composition may comprise between 0.001 g/L to 12 g/L of 2′-FL, preferably between 0.002 g/L to 10 g/L of 2′-FL, more preferably between 0.005 g/L to 5 g/L of 2′-FL.

The composition may comprise between 0.001 g/L to 5 g/L of DFL, preferably between 0.002 g/L to 4 g/L of DFL, more preferably between 4 g/L to 3 g/L of DFL.

The composition may comprise between 0.01 g/L to 6 g/L of LNT, preferably between 0.025 g/L to 5 g/L of LNT, more preferably between 0.05 g/L to 1 g/L of LNT.

The composition may comprise between 0.001 g/L to 2 g/L of 6′-SL, preferably between 0.002 g/L to 1.5 g/L of 6′-SL, more preferably between 0.005 g/L to 1 g/L of 6′-SL.

The composition may comprise between 0.01 g/L to 2 g/L of 3′-SL, preferably between 0.025 g/L to 1.5 g/L of 3′-SL, more preferably between 0.05 g/L to 1 g/L of 3′-SL.

The composition may comprise between 0.01 g/L to 7 g/L of 3-FL, preferably between 0.025 g/L to 6 g/L of 3-FL, more preferably between 0.05 g/L to 5 g/L of 3-FL.

In some embodiments, the composition comprises Bifidobacterium longum transitional microorganism preferentially utilizing 3-fucosyllactose (3-FL) over 2′-fucosyllactose (2′-FL) mixed with 3′-O-fucosyllactose (3-FL) and lacto-N-tetraose (LNT). The composition may comprise between 10³ to 10¹² cfu of probiotic strain, more preferably between 10⁷ and 10¹² cfu such as between 10⁸ and 10¹⁰ cfu of probiotic strain per g of composition on a dry weight basis mixed with 3-FI in an amount between 0.01 g/L to 7 g/L of 3-FL, preferably between 0.025 g/L to 6 g/L of 3-FL, more preferably between 0.05 g/L to 5 g/L of 3-FL and with LNT in an amount between 0.01 g/L to 6 g/L of LNT, preferably between 0.025 g/L to 5 g/L of LNT, more preferably between 0.05 g/L to 1 g/L of LNT.

Suitably, the 3′-O-fucosyllactose (3′FL) and lacto-N-tetraose (LNT) comprised in the composition promote the growth of a Bifidobacterium longum transitional microorganism that preferentially utilizes 3-fucosyllactose (3-FL) over 2′-fucosyllactose (2′-FL).

### Methods

In another aspect, the present invention provides a method for treating and/or preventing an allergy and/or allergic sensitization in an infant or young child; wherein the method comprises administered an effective amount of a Bifidobacterium longum transitional microorganism, a prebiotic or a combination of a Bifidobacterium longum transitional microorganism and a prebiotic to a subject in need thereof.

In a further aspect, the present invention relates to the use of Bifidobacterium longum transitional microorganism, a prebiotic or a combination of a Bifidobacterium longum transitional microorganism and a prebiotic for the preparation of a medicament for treating and/or preventing an allergy and/or allergic sensitization in an infant or young child.

The Bifidobacterium longum transitional microorganism may be a Bifidobacterium longum transitional microorganism as described herein.

The prebiotic may be a prebiotic as described herein.

The combination of a Bifidobacterium longum transitional microorganism and a prebiotic may be provided in any form as described herein. For example, the combination may be provided in a composition as described herein.

### EMBODIMENTS

The present invention provides the embodiments according to the following numbered clauses:
1. A Bifidobacterium longum transitional microorganism for use in treating and/or preventing an allergy and/or allergic sensitization in an infant or young child.
2. A prebiotic for use in treating and/or preventing an allergy and/or allergic sensitization in an infant or young child by promoting the growth and/or survival of a Bifidobacterium longum transitional microorganism in the gut of the infant or young child, wherein the prebiotic is:
   i. a glycan substrate, suitably selected from the group recited in any of Tables 1 to 3; and/or
   ii. a human milk oligosaccharide (HMO), suitably selected from the group consisting of 2′-O-fucosyllactose (2′-FL), 3′-O-fucosyllactose (3′-FL), lactodifucotetraose/difucosyllactose (di-FL), 3′-O-sialyllactose (3′-SL), 6′-O-sialyllactose (6′-SL), lacto-N-tetraose (LNT) and lacto-N-neotetraose (LNnT) and any combination thereof.
3. The Bifidobacterium longum transitional microorganism for use according to clause 1, wherein the Bifidobacterium longum transitional microorganism is used in combination with a prebiotic, and wherein the prebiotic is:
   i. a glycan substrate, suitably selected from the group recited in any of Tables 1 to 3; and/or
   ii. a human milk oligosaccharide (HMO), suitably selected from the group consisting of 2′-O-fucosyllactose (2′-FL), 3′-O-fucosyllactose (3′-FL), lactodifucotetraose/difucosyllactose (di-FL), 3′-O-sialyllactose (3′-SL), 6′-O-sialyllactose (6′-SL), lacto-N-tetraose (LNT) and lacto-N-neotetraose (LNnT) and any combination thereof.
4. The prebiotic for use according to clause 2, wherein the prebiotic is used in combination with a Bifidobacterium longum transitional microorganism.
5. A combination of a Bifidobacterium longum transitional microorganism and a prebiotic for use in treating and/or preventing an allergy and/or allergic sensitization in an infant or young child; wherein the prebiotic is:
   i. a glycan substrate, suitably selected from the group recited in any of Tables 1 to 3; and/or
   ii. a human milk oligosaccharide (HMO), suitably selected from the group consisting of 2′-O-fucosyllactose (2′-FL), 3′-O-fucosyllactose (3′-FL), lactodifucotetraose/difucosyllactose (di-FL), 3′-O-sialyllactose (3′-SL), 6′-O-sialyllactose (6′-SL), lacto-N-tetraose (LNT) and lacto-N-neotetraose (LNnT) and any combination thereof.
6. The Bifidobacterium longum transitional microorganism, prebiotic or combination for use according to any preceding clause, wherein preventing an allergy and/or allergic sensitization comprises reducing the risk of developing an allergy and/or allergic sensitization.
7. The Bifidobacterium longum transitional microorganism, prebiotic or combination for use according to any one of clauses 2-6, wherein the prebiotic is a glycan substrate selected from the group recited in any of Tables 1 to 3.
8. The Bifidobacterium longum transitional microorganism, prebiotic or combination for use according to any one of clauses 2-6, wherein the prebiotic is a HMO selected from the group consisting of 2′-FL, 3′-FL, di-FL, 3′-SL, 6′-SL, LNT and LNnT, and any combination thereof.
9. The Bifidobacterium longum transitional microorganism, prebiotic or combination for use according to any one of clauses 2-8, wherein the HMO is 3′-FL.
10. The Bifidobacterium longum transitional microorganism, prebiotic or combination for use according to any one of the preceding clauses, wherein the Bifidobacterium longum transitional microorganism is capable of metabolizing the HMO(s) and/or the glycan substrate(s).
11. The Bifidobacterium longum transitional microorganism, prebiotic or combination for use according to any one of the preceding clauses wherein the Bifidobacterium longum transitional microorganism preferentially utilizes 3′-fucosyllactose (3′-FL) over 2′-fucosyllactose (2′-FL).
12. The Bifidobacterium longum transitional microorganism, prebiotic or combination for use according to any one of the preceding clauses, wherein the Bifidobacterium longum transitional microorganism is capable of metabolizing a glycan substrate selected from the group recited in any of Tables 1 to 3.
13. The Bifidobacterium longum transitional microorganism, prebiotic or combination for use according to any one of the preceding clauses, wherein the Bifidobacterium longum transitional microorganism encodes one or more CAZymes selected from the group recited in Table 1, preferably wherein the Bifidobacterium longum transitional microorganism further encodes one or more CAZymes selected from Table 2 and 3.
14. The Bifidobacterium longum transitional microorganism, prebiotic or combination for use according to any one of the preceding clauses, wherein the Bifidobacterium longum microorganism has an Average Nucleotide Identity (ANI) of at least 98% with at least one Bifidobacterium longum strain selected in the group consisting of CNCM I-5683, CNCM I-5684, CNCM I-5685, CNCM I-5686, CNCM I-5687 and CMCC-P0001 (ATCC BAA-2753), and any combination thereof.
15. The Bifidobacterium longum transitional microorganism, prebiotic or combination for use according to any one of the preceding clauses, wherein the Bifidobacterium longum transitional microorganism and/or prebiotic increases the levels of IL-10 in the infant or young child.
16. The Bifidobacterium longum transitional microorganism, prebiotic or combination for use according to any one of the preceding clauses, wherein the Bifidobacterium longum transitional microorganism and/or prebiotic increases the IL-10/IL-12 ratio in the infant or young child.
17. The Bifidobacterium longum transitional microorganism, prebiotic or combination for use according to any one of the preceding clauses, wherein the Bifidobacterium longum transitional microorganism and/or prebiotic reduces IL-5 in the infant or young child.
18. The Bifidobacterium longum transitional microorganism, prebiotic or combination for use according to any one of the preceding clauses, wherein the Bifidobacterium longum transitional microorganism or prebiotic modulates the permeability of the gut epithelial barrier.
19. The Bifidobacterium longum transitional microorganism, prebiotic or combination for use according to any one of the preceding clauses, wherein the Bifidobacterium longum transitional microorganism or prebiotic decreases the permeability of the gut epithelial barrier.
20. The Bifidobacterium longum transitional microorganism, prebiotic or combination for use according to any one of the preceding clauses, wherein the Bifidobacterium longum transitional microorganism or prebiotic increases the levels of riboflavin and/or folic acid in the infant or young child.
21. The Bifidobacterium longum transitional microorganism, prebiotic or combination for use in treating an allergy and/or allergic sensitization in an infant or young child according to any one of the preceding clauses.
22 The Bifidobacterium longum transitional microorganism, prebiotic or combination for use in preventing an allergy and/or allergic sensitization in an infant or young child according to any one of the preceding clauses.
23. The Bifidobacterium longum transitional microorganism, prebiotic or combination for use in reducing the risk of developing an allergy and/or allergic sensitization in an infant or young child according to any one of clauses 1-21.
24. The Bifidobacterium longum transitional microorganism, prebiotic or combination for use according to any preceding clause; wherein the allergen is a food or a respiratory allergen or a skin allergen.
25. Use of a Bifidobacterium longum transitional microorganism, prebiotic or combination as defined in any one of the preceding clauses for promoting immune tolerance in an infant or young child, preferably by promoting the growth of a Bifidobacterium longum transitional microorganism in the gut of the infant or young child.
26. Use of a Bifidobacterium longum transitional microorganism, prebiotic or combination as defined in any one of the preceding clauses for modulating gut barrier permeability in an infant or young child, preferably by promoting the growth of a Bifidobacterium longum transitional microorganism in the gut of the infant or young child.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the figures and non-limiting examples.

### EXAMPLES

### Example 1: Transitional B. longum Increases Gut Epithelial Barrier Resistance

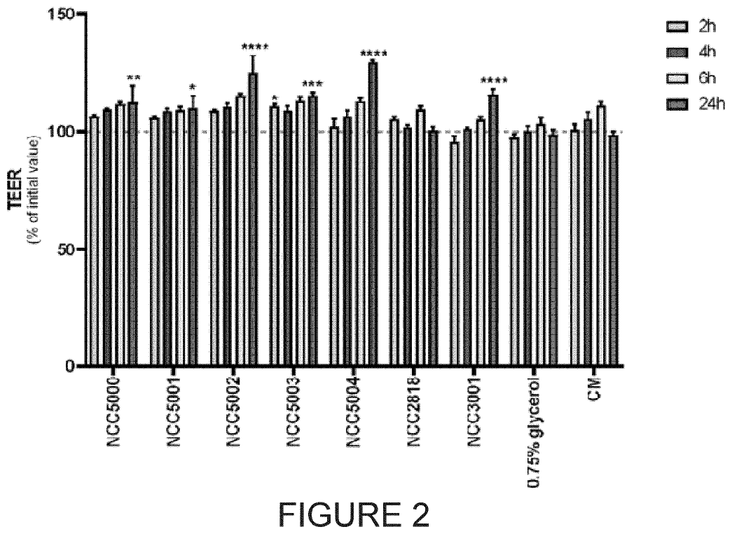

In vitro experiments using a human colorectal adenocarcinoma cell line (Caco-2) have shown that the transitional B. longum strains were able to increase the transepithelial electrical resistance (TEER) when incubated with the epithelial cells for 24 hours (see FIG. 2).

These results indicate that the transitional B longum play a role in decreasing the paracellular permeability during the transition from breast milk to solid food which is associated with an increase in dietary antigen intakes and thus contribute to the barrier equilibrium needed for the optimal maturation of the immune system.

### Example 2: Transitional B. longum Increases the Anti-Inflammatory Cytokine IL-10, Boosts IL-10/IL-12 Ratio and Reduces IL-5 Expression by T Helper Type 2 Skewed Cells

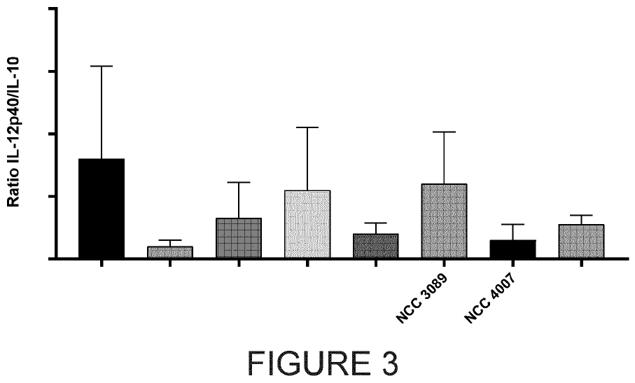

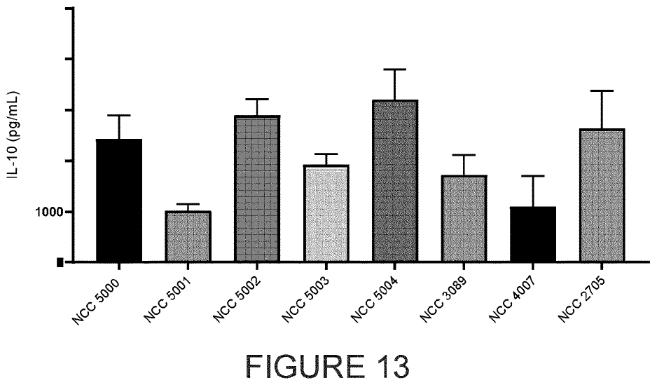

In vitro, B. longum transitional strains increased immune regulatory responses in human peripheral blood mononuclear cells, characterized by increase in interleukin 10 (IL-10) (FIG. 13) and IL-10/IL-12p40 ratio after 36 hours stimulation to a similar extent or even greater extent than probiotic Lactobacillus rhamnosus (NCC4007) (FIG. 3).

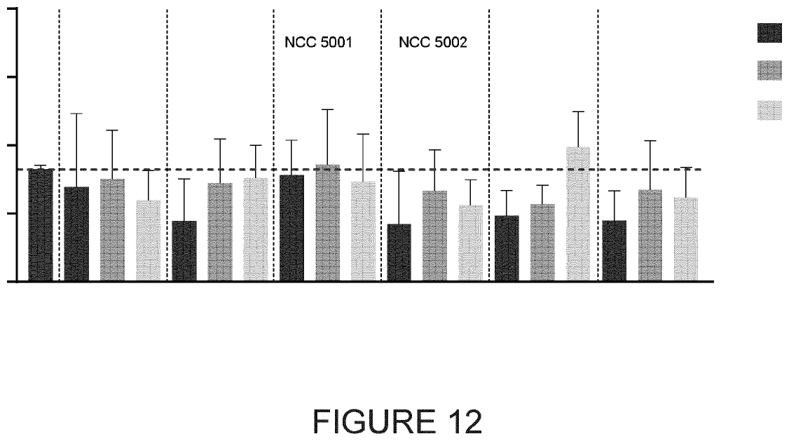

In addition, in vitro, B. longum transitional strains decreased IL-5 expressed by T helper type 2 skewed cells after 48 hours stimulation to a similar or even greater extent than probiotic B lactis (with known anti-inflammatory properties) (FIG. 12). Determination of IL-5 was performed according to the following method: Peripheral blood mononuclear cells (PBMC) were isolated from buffy coats obtained from healthy adults by density gradient. PBMC were then seeded at 1.5×10⁶ cells/ml in a 48-well bottom plate in complete Isocove's modified Dulbecco's medium (cIMDM) containing 10% fetal bovine serum, 1% glutamine, 1% penicillin/streptomycin and 0.1% gentamycin. Cells were cultured in presence of 50 ng/ml IL-4 and 1 ug/ml of anti-CD40. After 3 days of culture, different bacterial strains including all transitional B. longum isolates were added at the indicated concentrations. Cell culture supernatants were collected to assess cytokine expression for IL-5 by ELISA. Standard curve for each cytokine was used to calculate absolute amount (picogram/ml) from optical density readouts.

### Example 3: Analysis of Carbohydrate Active Enzyme (CAZyme) Genes of Bifidobacterium longum Transitional Microorganism

Genomes of Bifidobacterium longum subspecies listed in FIG. 4 were annotated to CAZymes combining dbCAN2 (Zhang et al., Nucleic Acids Res. 46 (W1): W95-W101 (2018)) tools and databases HMMdb (v9) and Diamond (v2.0.8). Query sequences with >0.50 coverage and e-value <1e-15 were annotated with HMMER according to the dbCAN CAZyme domain HMM database. Diamond was also used to annotate query sequences with hits in the CAZy database (Drula et al., Nucleic Acids Res. 50 (D1): D571-D577 (2022)) (http://www.cazy.org/) with >0.90 identity, and e value <1e-102. HMMER annotation was prioritized and used in instances of mismatched CAZyme annotations of query sequences between HMMER and DIAMOND tools. Only CAZyme families and subfamilies encoding Glycoside Hydrolases (GHs) and Polysaccharide Lyases (PLs) were used for comparative analyses of B. longum subspecies (see FIG. 4).

### Example 4: Utilization of Glycan Substrates

Pulverized or homogenized stool samples were mixed 10-fold by adding PBS/glycerol (1/10) (w/v) before centrifugation at 2000 g for 2 minutes. The slurry and pellet were then stored at −80° C. Frozen fecal samples were thawed from storage at −80° C. before centrifugation at 2000 g for 2 minutes. The resulting supernatant was inoculated with media based on that disclosed in Daguet et al. (Journal of Functional Foods; 2016; 20; 369-379). This media was supplemented with specific fibers to be tested at 5 g/L and a Bifidobacteria supplement of 5E07 CFU/ml.

The culture was set up at 37° C., N₂ gas flow to ensure anaerobic conditions and gentle stirring. Aliquots were taken and analyzed at the time points indicated.

B. longum transitional strain NCC 5001 growth is promoted by pectin (sugar beet) and arabinogalactan (larch wood) (FIG. 5).

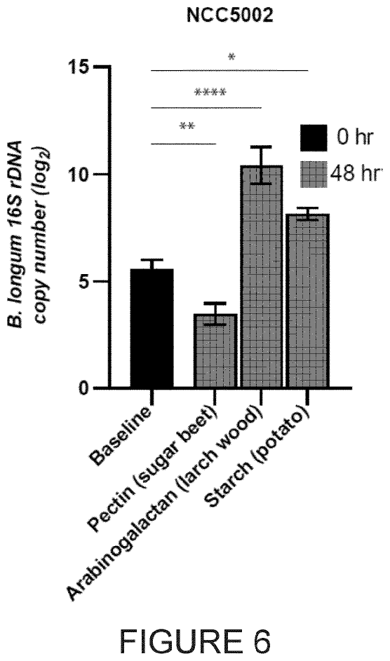

B. longum transitional strain NCC 5002 growth is promoted by arabinogalactan (larch wood) and starch (potato) (FIG. 6).

### Example 5: Characterization of B. Longum Transitional Microorganism

B. longum transitional strains were isolated from the feces of breast-fed infants using Eugon Tomato Agar (ETA). Obtained isolates were sequenced using PacBio to obtain a fully closed assembled genome for each of the strain. Each strain was deposited in the internal Nestlé Culture Collection (NCC, Lausanne, Switzerland) and at the Collection Nationale de Microorganisms (CNCM) at the Pasteur Institute (Paris, France) together with their genome sequence data. The genome of the strains was compared by Average Nucleotide Identity (ANI) using OrthoAni (https://www.ezbiocloud.net/tools/orthoani) to other publicly available genomes representing the overall diversity of the B. longum species (Table 5), and to the Metagenomic Assembled Genomes (MAG) obtained from metagenomic sequences issued from infant feces of the same cohort.

TABLE 5
list of genomes used for ANI analysis and their publicly available references.

| | | Nestlé Culture | |
|---|---|---|---|
| | | Collection | |
| Taxonomy | Strain number | internal number | Genome reference |
| B. longum | CNCM I-5683 | NCC 5000 | Available at the CNCM |
| B. longum | CNCM I-5684 | NCC 5001 | Available at the CNCM |
| B. longum | CNCM I-5685 | NCC 5002 | Available at the CNCM |
| B. longum | CNCM I-5686 | NCC 5003 | Available at the CNCM |
| B. longum | CNCM I-5687 | NCC 5004 | Available at the CNCM |
| B. longum subsp. suis | BSM11-5 | NA | GCF_001870705.1 |
| B. longum subsp. infantis | 3_mod | NA | GCF_902167615.1 |
| B. longum subsp. longum | JDM301 | NA | CP002010 |
| B. longum | BXY01 | NA | GCF_000730205.1 |
| B. longum subsp. longum | CMCC_P0001 | NA | GCF_000410595.1 |
| B. longum subsp suillum | SU-851 | NA | GCF_016882605.1 |
| B. longum subsp suillum | JCM19995 (T) | NCC 3079 | GCF_017132755 |
| B. longum subsp. suis | 2074B | NA | GCF_016759645.1 |
| B. longum subsp. suis | Su859 (T) | NA | GCF_900103055.1 |
| B. longum subsp. suis | DSM-20211 (T) | NA | GCF_000771285.1 |
| B. longum subsp. suis | LMG_21814 (T) | NA | GCF_000741625.1 |
| B. longum subsp. suis | 209B | NA | GCF_016759725.1 |
| B. longum subsp. suis | UMA026 | NA | PHUM01000001 |
| B. longum subsp. suis | AGR2137 | NA | GCF_000421385.1 |
| B. longum subsp. longum | NCC 2075 | NCC 2705 | GCF_000007525.1 |
| B. longum subsp. longum | DJO10A | NA | GCF_000008945.1 |
| B. longum subsp. longum | 157F | NA | GCF_000196575.1 |
| B. longum subsp. longum | DSM_20219 (T) | NA | GCF_900104835.1 |
| B. longum subsp. longum | JCM_1217 | NA | GCF_000196555.1 |
| B. longum subsp infantis | ATCC_15697 (T) | NA | JDTT01000001 |
| B. longum subsp infantis | Bi-26 | NA | GCF_004919065.2 |
| B. longum subsp infantis | JCM_11347 | NA | CP062951 |

(T) stands for typestrain.

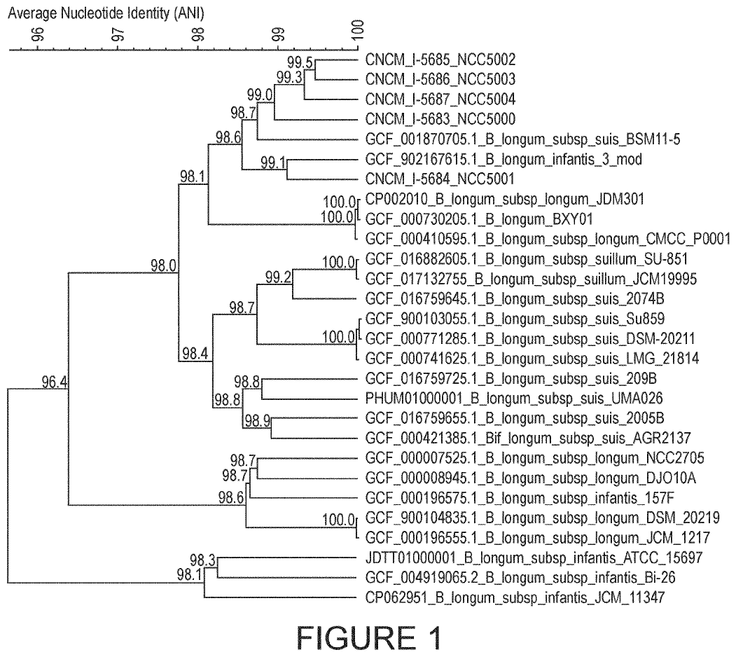

The analysis demonstrates that the newly described strains group together with the MAGs obtained from the same cohort, defining a well delineated clade belonging to the B. longum species. Two previously isolated strains BSM11-5 and 3_mod are found to be grouped within this newly described clade. The clade is genetically different from B. longum subspecies longum (96.40% ANI) subspecies. The clade is related, while still clearly distinct, to B. longum subspecies. suis/suillum (98.207%), and to the group of strains (JDM301, CMCC_P0001 and BXY01) previously suggested to be a new B. longum subspecies (O'Callaghan et al. 2015), sharing an identity of 98.260% to this group of strains. FIG. 1 shows ANI UPGMA based phylogenetic tree. The scale represents the percentage (%) of identity at each branch point.

A selection of the above mentioned genomes, representing the diversity of the B. longum subspecies, were annotated for Carbohydrate-Active enZYmes (CAZY) using the dbCAN annotation pipeline (http://bcb.unl.edu/dbCAN/). Results showed that B. longum subsp. longum, B. longum subsp. suis and B. longum subsp. suillum strains contained a GH20 (lacto-N-biosidase) enzyme, implicated in the degradation and metabolization of Lacto-N-tetraose (LNT). Similarly to B. longum subsp. infantis strains, B. longum transitional strains also possessed a similar enzyme, and in addition harbored GH29 (fucosidase) encoding genes which are implicated in the degradation and metabolization of fucosylated human milk oligo-saccharides, such as 2′FL, 3′FL or diFL. Additionally, three of the strains (CNCM I-5684, BSM1-15& 3_mod) also harbor a GH 33 (sialidase) encoding gene implicated in the degradation and metabolization of sialilated HMO such as 3′SL or 6′SL (Table 6).

TABLE 6
Number of genes encoding for GH20 (lacto-N-biosidase), GH29 (α-fucosidase),
GH95 ((α-fucosidase/(α-galactosidase) and GH33 (sialidase) glucohydrosylhydrase
family enzymes in each of the represented genomes.

| | | No of | No of | No of | No of |
|---|---|---|---|---|---|
| | | predicted | predicted | predicted | predicted |
| | | GH20 | GH29 | GH95 | GH33 |
| | Genome | encoding | encoding | encoding | encoding |
| CAZYmes_pred | reference | genes | genes | genes | genes |
| B. longum subsp. Longum | GCF_900104835.1 | 1 | 0 | 0 | 0 |
| DSM 20219 (T) | | | | | |
| B. longum subsp. longum | GCF_000007525.1 | 1 | 0 | 0 | 0 |
| NCC 2705 | | | | | |
| B. longum subsp infantis | JDTT01000001 | 3 | 3 | 1 | 2 |
| ATCC 15697 (T) | | | | | |
| B. longum subsp infantis | Nestlé | 3 | 1 | 1 | 2 |
| LMG 11588 | internal | | | | |
| B. longum subsp. suis | GCF_000771285.1 | 1 | 0 | 1 | 1 |
| DSM 20211 (T) | | | | | |
| B. longum subsp. suillum | GCF_017132755 | 1 | 0 | 1 | 0 |
| JCM 19995 (T) | | | | | |
| B. longum spp CNCM | Available at | 1 | 1 | 1 | 0 |
| I-5683 (NCC 5000) | the CNCM | | | | |
| B. longum spp CNCM | Available at | 3 | 2 | 1 | 2 |
| I-5684 (NCC 5001) | the CNCM | | | | |
| B. longum spp CNCM | Available at | 2 | 1 | 1 | 0 |
| I-5685 (NCC 5002) | the CNCM | | | | |
| B. longum spp CNCM | Available at | 1 | 1 | 1 | 0 |
| I-5686 (NCC 5003) | the CNCM | | | | |
| B. longum spp CNCM | Available at | 1 | 1 | 1 | 0 |
| I-5687 (NCC 5004) | the CNCM | | | | |
| B. longum spp. BSM1-15 | GCF_001870705.1 | 1 | 1 | 1 | 1 |
| B. longum spp. 3 mod | GCF_902167615.1 | 3 | 2 | 1 | 2 |
| B. longum spp. JDM301 | CP002010 | 1 | 1 | 1 | 0 |

All newly obtained genomes were compared and aligned with the genome of two strains (B. longum subsp. infantis ATCC15697 and B. kashiwanohense DSM 21854) belonging to species for which the genes responsible for fucosylated HMOs utilization were elucidated (James et al. 2019).

### Results

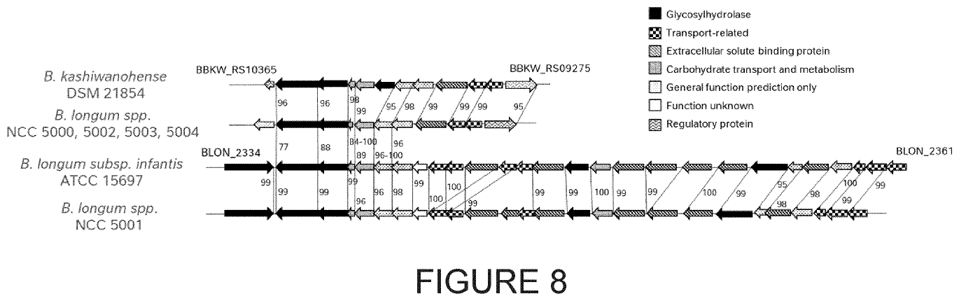

As shown in FIG. 8, all newly described strains contained genes responsible for the utilization of fucosylated HMOs. While NCC 5001 organization reflects the one of B. longum subsp. Infantis ATCC 15697, all other strains (NCC 5000, NCC 5002, NCC 5003, NCC 5004) harbor a gene organization closer to that of B. kashiwanohense DSM 21854. Overall, the similarity to the well described fucosidases of B. longum subsp. infantis ATCC 15697 is above 77% (for BLON_2334) and 88% (BLON_2335) in all newly described strains.

### Example 6: Utilization of Fucosylated HMOs

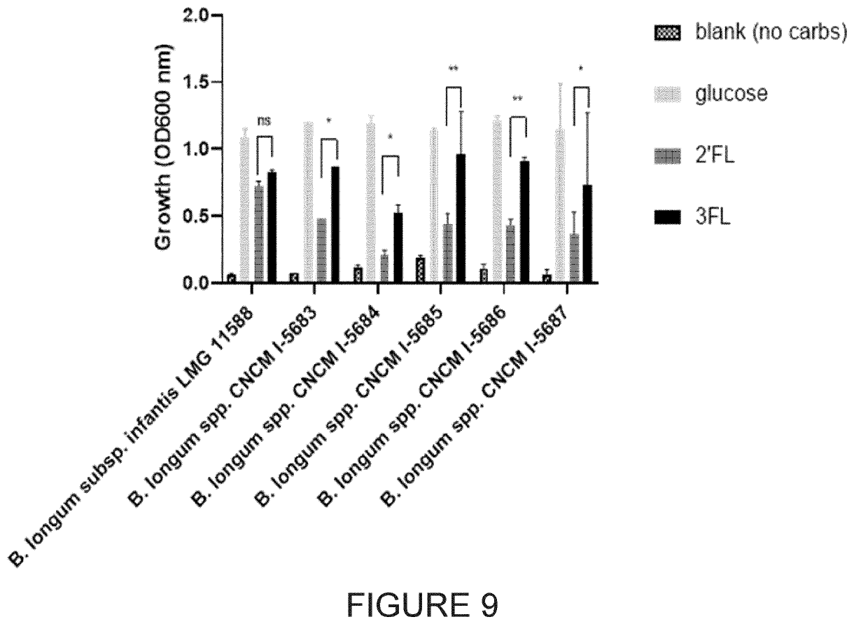

All strains retrieved from the Nestle Culture Collection (Table 7) were reactivated from a freeze-dried stock, using two successive culturing steps (16 h, 37° C., anaerobiosis) in MRS supplemented with 0.05% cysteine (MRSc). Reactivated cultures were then centrifuged, washed and resuspended in 1 volume of PBS. Washed cells were used to inoculate MRS based medium without a carbon source (MRSc-C) (10 g I-1 of bacto proteose peptone n°3, 5 g I-1 bacto yeast extract, 1 g I-1 Tween 80, 2 g I-1 di-ammonium hydrogen citrate, 5 g I-1 sodium acetate, 0.1 g I-1 magnesium sulphate, 0.05 g I-1 manganese sulfate, 2 g I-1 di-sodium phosphate, 0.5 g I-1 cysteine) in which glucose, 2′FL or 3′FL were added as unique carbon source at a concentration of 0.5%. Growth was then performed in a 96 well microplate, with a volume of 200 μl per well. Incubation was performed in anaerobiosis for 48 h, and optical density was measured in a spectrophotometer at 600 nm. As shown in FIG. 9, all B. longum transitional strains grew on fucosylated HMOS.

### Results

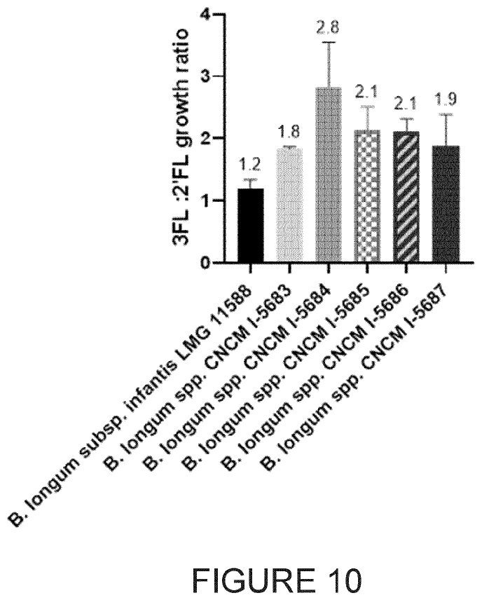

All B. longum transitional strains grew better on 3′FL than 2′FL and reached a higher cell density on this carbohydrate. This behavior indicates a preference for 3′FL over 2′FL (ratios from 1.8 to 2.8-see FIG. 10), which is not observed in B. longum subsp. infantis LMG 11588.

TABLE 7
list of the strains (and corresponding numbers) used
for the individual fucosylated HMO growth studies

| | Internationally | Nestlé Culture |
|---|---|---|
| | recognized | Collection |
| Taxonomy | deposit number | internal number |
| Bifidobacterium longum | LMG 11588 | NCC 3089 |
| subsp. infantis | | |
| Bifidobacterium longum | CNCM I-5683 | NCC 5000 |
| Bifidobacterium longum | CNCM I-5684 | NCC 5001 |
| Bifidobacterium longum | CNCM I-5685 | NCC 5002 |
| Bifidobacterium longum | CNCM I-5686 | NCC 5003 |
| Bifidobacterium longum | CNCM I-5687 | NCC 5004 |

### Methods

### CACO-2 Cells Culture and Transepithelial Electrical Resistance Measurement

Caco-2 cells (HTB-37; American Type Culture Collection) were seeded in 24-well semi-permeable inserts. Caco-2 monolayers were cultured for 14 days, with three medium changes/week, until a functional cell monolayer with a transepithelial electrical resistance (TEER) was obtained. Cells were maintained in Dulbecco's Modified Eagle Medium (DMEM) containing glucose and glutamine and supplemented with HEPES and 20% (v/v) heat-inactivated fetal bovine serum. Before addition of bacteria to the apical compartment, the TEER of the Caco-2 monolayers was measured (=0 h time point). The TEER of an empty insert was subtracted from all readings to account for the residual electrical resistance of an insert. Then, probiotic strains (directly taken from a glycerol stock) were diluted in Caco-2 complete medium and apically added to the Caco-2-bearing inserts at 2×10E6 colony-forming unit. Cells were also exposed to Caco-2 complete medium (CM) in both chambers as control and to 0.75% glycerol in the apical compartment as vehicle control. Cells were treated for 24 h and TEER was measured at several time points (2 h, 4 h, 6 h and 24 h). After subtracting the TEER of the empty insert, all timepoint values were normalized to its own Oh value (to account for the differences in initial TEER of the different inserts) and are presented as percentage of initial value (FIG. 1).

Immunoprofiling with PBMC Cells

Peripheral blood mononuclear cells (PBMC) were isolated from buffy coats obtained from healthy adults by density gradient. PBMC were then seeded at 1.5×10⁶ cells/ml in a 48-well bottom plate in complete Isocove's modified Dulbecco's medium (cIMDM) containing 10% fetal bovine serum, 1% glutamine, 1% penicillin/streptomycin and 0.1% gentamycin. PBMC were stimulated for 36 hours in the presence of different bacterial strains including all transitional B longum isolates at 10⁷ CFU/ml and probiotic strains. Cell culture supernatants were collected to assess cytokine expression for IL-10 and IL-12p40 by ELISA. Standard curve for each cytokine was used to calculate absolute amount (picogram/ml) from optical density readouts.

### Example 7: B. longum Transitional Microorganisms Produces Precursor of Folate and Riboflavin, Two Metabolites that Maintain Gut Barrier Integrity

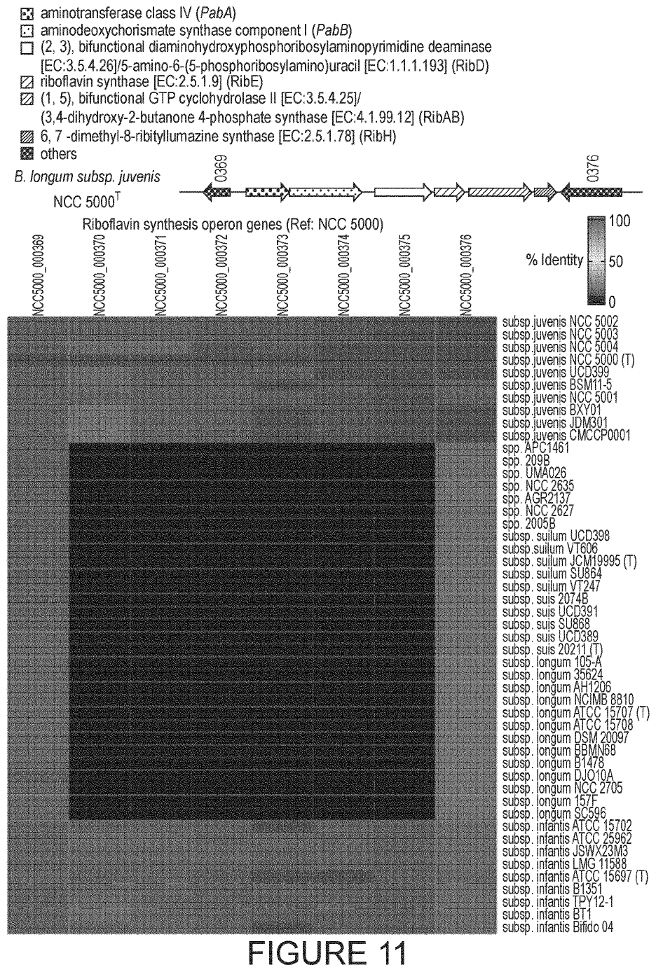

The genetic characterization of the B. longum transitional isolates showed that they harbour a particular genetic region containing a set of 6 genes (FIG. 11). As shown, B longum transitional and B. longum subsp. infantis strains are the only ones to harbour this set of genes. The whole operon is absent from strains belonging to B. longum subsp. longum, B. longum subsp. suis and B. longum subsp. suillum. This region encompasses two genes (PabA and PabB) implicated in the conversion of chorismic acid to 4-amino-4-deoxychorismate, a precursor of p-aminobenzoate and folic acid in different microorganisms. As well, this region harbored another set of 4 genes (RibD, RibE, RibAB, RibH) implicated in the biosynthesis of riboflavin, as recently demonstrated in B. longum subsp. infantis (Solopova et al., 2020, Front Microbiol, 2020. 11: p. 573335).

The presence of these genes indicates that B longum transitional microorganisms may be capable of producing riboflavin and folic acid.

### Example 8 B. longum Transitional Microorganism Produces SCFAs Beneficial for Allergy Prevention and Management

Methods: In-vitro batch fermentations of 3-fucosylactose (3FL) containing infant microbiome with or without the supplementation of Bifidobacterium longum spp infantis or Bifidobacterium longum transitional were caried out for 48 h. Acetate, butyrate, propionate were measured from supernatants collected at T0, T24 and T48 h by 1H-NMR technique. Total SCFAs corresponds to the sum of the peak integrals of acetate, butyrate, and propionate. The bar plot indicates the dynamic of consumption and production of total SCFAs between 0 and 24 h (blue bar) and 24 and 48 h (red bar).

### Results:

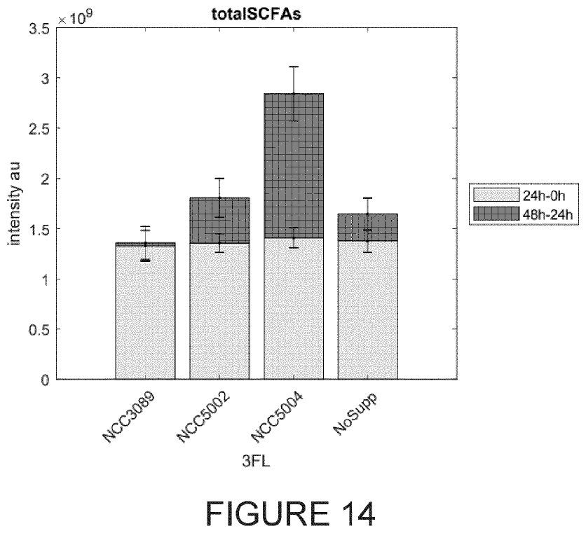

FIG. 14 shows that Bifidobacterium longum transitional is more metabolically active on weaning relevant HMO (i.e 3FL) than B. I. infantis and produce more total SCFAs which is beneficial for allergy prevention and management as shown in a number of studies (Trompette et al. Mucosal Immunology 15, 908-926 (2022); Roduit et al. Allergy, April; 74 (4): 799-809 (2019); Canani et al. Sci Rep August 21; 8 (1): 12500 (2018): Gio-Batta, et al. Sci Rep 10, 22449 (2020): Cait et al. 2019 J Allergy Clin Immunol. 2019 December; 144 (6): 1638-1647.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1: A method of microorganism for use in treating and/or preventing an allergy and/or allergic sensitization in an infant or young child, the method comprising administering a Bifidobacterium longum transitional microorganism to the infant or young child.

2: A method of treating and/or preventing an allergy in an infant or young child by promoting growth and/or survival of a Bifidobacterium longum transitional microorganism in the gut of the infant or young child, the method comprising administering a prebiotic to the infant or young child, wherein the prebiotic is:
i. a glycan substrate; and/or
ii. a human milk oligosaccharide (HMO).

3: The method according to claim 1, wherein the Bifidobacterium longum transitional microorganism is administered in combination with a prebiotic, and wherein the prebiotic is:
i. a glycan substrate; and/or
ii. a human milk oligosaccharide (HMO).

4: The method according to claim 2, wherein the prebiotic is administered in combination with a Bifidobacterium longum transitional microorganism.

5: A combination of a Bifidobacterium longum transitional microorganism and a prebiotic for use in treating and/or preventing an allergy and/or allergic sensitization in an infant or young child, wherein the prebiotic is:
i. a glycan substrate; and/or
ii. a human milk oligosaccharide (HMO).

6: The method of claim 2, wherein the prebiotic is a glycan substrate selected from the group consisting of those disclosed in any of Tables 1 to 3.

7: The method of claim 2, wherein the prebiotic is a HMO selected from the group consisting of 2′-O-fucosyllactose (2′-FL), 3′-O-fucosyllactose (3′-FL), lactodifucotetraose/difucosyllactose (di-FL), 3′-O-sialyllactose (3′-SL), 6′-O-sialyllactose (6′-SL), lacto-N-tetraose (LNT), lacto-N-neotetraose (LNnT), and any combination thereof.

8: The method of claim 2, wherein the HMO is 3′-FL.

9: The method of claim 3, wherein the Bifidobacterium longum transitional microorganism is capable of metabolizing the HMO(s) and/or the glycan substrate(s).

10: The method of claim 1 wherein the Bifidobacterium longum transitional microorganism preferentially utilizes 3′-fucosyllactose (3′-FL) over 2′-fucosyllactose (2′-FL).

11: The method of claim 1, wherein the Bifidobacterium longum transitional microorganism: (a) is capable of metabolizing a glycan substrate selected from the group consisting of those disclosed in any of Tables 1 to 3; and/or (b) encodes one or more CAZymes selected from the group consisting of those disclosed recited in Table 1.

12: The method of claim 1, wherein the Bifidobacterium longum microorganism has an Average Nucleotide Identity (ANI) of at least 98% with at least one Bifidobacterium longum strain selected in the group consisting of CNCM I-5683, CNCM I-5684, CNCM I-5685, CNCM I-5686, CNCM I-5687, CMCC-P0001 (ATCC BAA-2753), and any combination thereof.

13: The method of claim 1, wherein the Bifidobacterium longum transitional microorganism:
(a) increases the levels of IL-10 in the infant or young child;
(b) increases the IL-10/IL-12 ratio in the infant or young child;
(c) reduces the levels of IL-5 in the infant or young child;
(d) modulates the permeability of the gut epithelial barrier in the infant or young child; and/or
(e) increases the levels of riboflavin and/or folic acid in the infant or young child.

14: A method for promoting immune tolerance in an infant or young child, the method comprising administering at least one of a Bifidobacterium longum transitional microorganism, a glycan substrate, or a human milk oligosaccharide (HMO) to the infant or young child.

15: A method for modulating gut barrier permeability in an infant or young child, the method comprising administering at least one of a Bifidobacterium longum transitional microorganism, a glycan substrate, or a human milk oligosaccharide (HMO) to the infant or young child.

16: The method of claim 3, wherein the glycan substrate is selected from the group consisting of those disclosed in any of Tables 1 to 3.

17: The method of claim 3, wherein the prebiotic is a HMO selected from the group consisting of 2′-O-fucosyllactose (2′-FL), 3′-O-fucosyllactose (3′-FL), lactodifucotetraose/difucosyllactose (di-FL), 3′-O-sialyllactose (3′-SL), 6′-O-sialyllactose (6′-SL), lacto-N-tetraose (LNT), lacto-N-neotetraose (LNnT), and any combination thereof.

18: The method of claim 3, wherein the HMO is 3′-FL.

19: The combination according to claim 5, wherein the glycan substrate is selected from the group consisting of those disclosed in any of Tables 1 to 3.

20: The combination according to claim 5, wherein the prebiotic is a HMO selected from the group consisting of 2′-O-fucosyllactose (2′-FL), 3′-O-fucosyllactose (3′-FL), lactodifucotetraose/difucosyllactose (di-FL), 3′-O-sialyllactose (3′-SL), 6′-O-sialyllactose (6′-SL), lacto-N-tetraose (LNT), lacto-N-neotetraose (LNnT), and any combination thereof.


SEQUENCE LISTING (64 sequences)

SEQ ID NO: 1 - protein, 676 aa, unidentified
MHWKGKIPMEQQISALEVADWTPAELAMPCPDYPVYQDDDLGAFPDAKGTTFRVWAPTASGVTLRLFTAGSPETAPEPETQADLTPGADGTWLARCDGIGHGVYYDYLVRFPDGTVNRTADPWARAAGVNGRRSMVVDLSKTNPDGWAEDRRPHIPSRDLVIWETHIGDFSNDEHSGIPEEHRGTYLAFTHPDTSVDGAGEFPTCVAYLKKLGITAVQIMPFYDYGSIDESIPRTDPKHGFNWGYDPLNYNVPEGSYSTDPYDGTVRIRECKQMIQALHAAGIKVIMDVVYNHMFSTDNWFERMIPGYALRRRADGTFADGSACTNDVATEHPMMRKYIVDSAVYWAREYHVDGFRFDLMGLIDVDTMNAVRTALDQLPGGATISMHGEPWAARETALVPGTILADKRCMPYLSPRIGAFCDSTRDAVRGHVFYQWVPGYLTGAAADYADDIRHAEDGWRGTLRETASVGQVIQYVSAHDDLTLWDKLCAAMRRIPTDTDYSAEGEACGDLMRANRLAAGIVFASAGVPFMLSGEEFARTKHGVSDSFSSPAALNQLDWRRAQRMGDLVDHYAALIQLRRGNPAYFGGARMIVPRDDETVVFRVGDDCVAINPTGSPQTQLTTDLEIPGPYGEVSEIAPNEWQCIYCSGSDMPGAVEQRLLPLPPYTFTVWRRNRG

SEQ ID NO: 2 - protein, 301 aa, unidentified
MPGDITGPSHLGVYGPCGFIRCVIEKVKKTARLGSESSAFDGRVLLGLDRLGLNPVSQRHVTSGSGTAGNTYDSAGNFPAVGYTKGDFHTCAKNIADYSNADEVQNCRLSGLQDLDTSQDHVQDTIADYLATLLDMGVYGFRVDAVKHIAAQDVAAIKAKLAAKTGRDVDGIFFEQEVIGNGSEVASIQPVNYLATGKVSESTYNKRLPAVFGGSITVAAGGLDAIGGPTWVDSDKAAVWVTNWDTERNGSSLTTRDGTSYLLANAFMLAYDYGQPHIFSGYYYASVDDGAPGAPAPPCRT

SEQ ID NO: 3 - protein, 448 aa, unidentified
MTTWIATTQDNRLVDRSGEVTATGATTAPDLQLDGNEYQALRGFGGCFNELGWLPLQTVTEAERDQIIKELFDPEEMNFAFNRAPVGANDFADHWYSYDEVDGDYDMEHFSVERDEETLIPYIHRAQEWQPDMKLFSSPWSPPTWMKRPKAYNYGRLVQTPENLKAYAKYLARYVREYAKHGIEVTQLHVQNEVFADQKFPSALWDAEALKVFIRDYLGPEFEAAGLDTDIWLGTLNGPEDMAWTGGGYGMRLNNYSRFVDNILFDDGARKYIKGIAYQWAGQNCIARTHESWPEIELIQSESECGDGQNTWEYAEYVFHLINHYLKNGATAYTYWNMILDDRDSTWGWWQNSLFTITADTHEVRRNPEYYVMRHFSQYVRPGAKVLGTTGHFNSMAIAFRNPDGTIAVVVQNALETAMPFEFADPADASRGIKATLAPRSFNTFVID

SEQ ID NO: 4 - protein, 369 aa, unidentified
LFAECGYNQLEIDARVAQVWHEIFEGPNKFYWETENGLGYVMDTGNNDVRTEGMSYAMMLAVQYGRQDVFDRLWGWVMKHMYMMDGHHAHYFSWSVSPDGSDRADGPAPDGEEYFAMDLFLADRRWGSSGIHDYAAQARELLHYCVHKGRDEDGEPMWNADNKLIKFIPETEWSDPSYHLPHFYEVFAECADGQDRAFWSEAASASRAYLKTACNAYTGMNGEYALYDGTPYTEERFGRHDWFYSDAYRTVANIGLDASWNNVPDVALCDRAAALQRFFLTHDRTCVYEVDGTPVDERVLHPVGFLAATAQGSLAAMHSVSEPDAVRNARAWVHMLWNTPMRVGRRRYYDNFLYAFAILALSGQYRREW

SEQ ID NO: 5 - protein, 915 aa, unidentified
MSRFVAFDHNRLLIDGRPRVLMCASLFPFRVPREQWKQRLQAVKSIGYHAIDVYIPWNFHETEPDIWNFEGQHDIEEFLRLTHEVGLYALVRPGPYICSEWDGGAIPAWVSTNGKTHVRQYDDAFLGEVRKWYGKIMPIIARQQYRGADADDGGVVILVQADNELDFYACHEPGKYIGALASMMRSYGVDVPIIACAGQGDMLRAGGTAEDVSPAVNLYPGDNDVDVDAQVRYYRRAADTYDVPLVVTETNRWHRTLRRLVGNGARFVGPFLQVSGWDFDYGTSVNNWGRVEAYMTHDYDFGGVIDPTGQERPDADDARRLCSIIDALGERLASATISDDRLTGIHLDSAEGDRFDETRVGVAVGSLDLHGGGRLLTFTNVSDDAADIRIGDAPDDALSDGGYPALTLPAGAGAMVVAGLPLGDGMMLRATSGELIELDESGHTLTLSAPTMAPGRFGAIIRPAHGIKADDIHIHTDALTAHMVNGDIVVQGDHGTATIGQGQSSWRITFTQPEHHVVLGCAPHSSVRSVAISLDWPSWTLCDGDVLHDALPLEDMGVYSGAGRYRAATNMRGALGVVLHNAADTVNVRCGNSGTDWQACGGVDAWIPFNEPVADDTKDIVVTTRIWGHSNFDDTRLNALRLNGKRGMCGAMSIERVLDIGSGWRVGYNRTDPDNAERLRENGLVLGDAPVPRCGFGSRSTTAWPHALVYTRTLSLGGASACLHIEHGQTRCEVRVGGRTIGIITPLAPTLWLGEVPDGSELSIVVNRMWGEDAGRIALLVGDELTGWTCESQGLKELRASCARATYVRASLPVLVPGGEGRWIRVPGASIRGSEYSNNTIVRFDGEGLQLTAFTDEYCLGRVVLGPLPGTVFAGGRGDLFVVPHGEGDLMLYAESTRERGGTLRGIMLGGSVDR

SEQ ID NO: 6 - protein, 687 aa, unidentified
MNDNMVDGGRTLTCSAGKALAEIFDRKTMPAAELFRQAEFLALRLRGCDEYSMCLRLEFYEAGQRERGADPTMYALFGLLPGVETTVYFDLRWLDGQTLFAPRTPGRLKMTIFGKPLRKADVSSIVLSSDASCDGRRLKVWPVQWADVIAHADPPVHALIDEMGQWKPRQWPGKTTNRAACDEALHRLAEPMPSGAGGMAGLPALLNRDSYGGCQDVHAEATGWFRVWREPGDDGRFWLLDPDGNAFISVGADCVNPGIDTRVDPVLPWVDRHVRELVDAARRNERIGDPDFSIRSAPNTYCGFECSYDYAMANLRHAFGASAWDAWKRIVLRHFHDWGMNTIGNWSDHELMRTAGIPYVLPADTLREYPRTKTMVFRDFPDVFSTEYERNADVYAQGLKDWRDDRNMIGYFMRNEPNWAFVYDLNLAEEMLANPERLASKEALVAWLVDRYHGDAQAWKQAWRIPDDSGIECFEDLIACPRYRCVRQMSGSMHDLRDFSILMIDRYVRIPAEALRRVDPHHLNLGMRYAYITDPTLLAGSDCYDVFSINSYQMTCYHQVDELGKTLNMPVMVGEFHHGALDRGLNAHGIRGVRTQEDRGKAYRYYVEQALRSRYFVGAHYFQYNDQSALGRFDGENYQIGLVDVCSQEYPEMAQAMRRCHVGMYDVAMGRKPPYDACPEEVAPIHY

SEQ ID NO: 7 - protein, 713 aa, unidentified
MTATISNGASASYSPAEDELGAADPTALLAESGDLKPLAERTYTNPVPYADGKSHTAPDPFVLKYRDLYYCYATDEHGILVSTSPDMVHWTSHGFCYTEAGRRNFWAPSVILINGVFHMYFSNMPAEETDTHTEIMRVAVSEDPLGPFEKKAELFNTFAIDSQVVYGDDGQLYLLYADNQVTGLSDDRPGTSVMIDRLVTPYSRENKPRPLIVPTMDEEIFARNRFGDGRDWHTVEGATYFAYRDRAFITYSANAYEHEDYFVGYSYAQLPNKQADAHIDQLDWTKQLNENRFDPLLIRSPKVEGTGHNSIVKAPNAVDDWIVYHGRNADDELYVGTEQRVMRIDPLYYAEGGLDTPGPTAAAQSAPLYGTVHDDFADGLNAGWSVISGAAHTESDVDGHALVADESSVFIAVSGKSSATQVIDVWAKAPVTPLGARFGIVVRYQDANNLTKLEVDAGRQVISVVDVIGGVASERVTNADLHDFDSHAWHEYRLERRYCRLEIRIDGRFAASCTISDKPGRAGLFSLRTGAAFSAYAATEHVNLWGAGLRDLGRELHADRRLVIDGGVRSSGVCPVTLELAYPLVSNRFVLDFAGQTSRGQALLSLGEYRLSGTASSVEFMRNGKSLPSTPEPARLRVFEDNVRRDRSGRAVLTIRIEALNGTMRLHLRGKTWQVPFADNAARARITLDRASLTGYERTSLESSIEERSASGN

SEQ ID NO: 8 - protein, 851 aa, unidentified
LATHPGMNPAAVIQGDRWRIGIITESLVRLEWQDNGKFEDHATQMIVNRDWLSDDANGADGANRADGTSNPPKFTKTERDGLLIIDTPALRLTYDMQPFSKEGLSIVVKGVANSQMNTWHYGEAQDGNLRGTARTLDAVDGEIELGLGVISRDGWAVLDDSASNVIVEGAEAATVKGEANPFGMWVIPREHPGKDLYVFGYGHRYIEAVQDFYKLTGPTPLLPRFALGNWWSRYHRYTEAEYLELVDRFEQEGLPFTTAVIDMDWHLVDNVDPKYGSGWTGYTWNREFFPDPERFQRILHEHGLRTTLNVHPRDGVRAFEDGYAKVAEHMGIDPAGGEPVEFDLTSPRFMEAYFDLHHGLETLGTDFWWLDWQQGGVTRQKGLDPLWMLNHMHYLDSARDGRWPLTFSRYAGPGSHRYPVGFSGDTVVTWESLKFQPYFTATASNIGYGWWSHDIGGHMFGYRDEELEARWYQLGAFSPINRLHSTDSPFNGKEPWNFRPETECSMDDALRLRHELLPYLYTMNWRAAAEGRPLVEPMYWQSPENVTAYGVEHEFRFGTELIVAPIVTPSDRNAQLAKTDVWLPQGTWFDFFTGRRYEAGSAEGRKFEAWRGLDGIPVFAKAGGIVPLQPLDEELNGSLNSVDNPRYLEVAVFPGANGAFDLKEDDGTNQAQPSDSALAVTHMDFRWEADGAVNAFEIAPVAGNVAAVPQQRTWTVTFTGVAPVAVDELIVTVGGAEVVPDVSYDTEALSLSVTVADAPVTQAVSIHATNGLRYADDPAIEDAFTVLYNAQIDYLAKERAYRLIKADGARALNALHTLESTKSSYPGHEFDDSHMPESVIQAIAEPLLRGL

SEQ ID NO: 9 - protein, 1943 aa, unidentified
MHHSTRKRWLASIGAVAAVATLATGGAVTAQAADAPVIKNADVAYPSFKGSDDPMKTAANNTTYNPAVSYLQETFDNDVKNLAGIDTDHDFWIDKILTRTGAQPTGKGTNDKGAYSYEGSDGNNYLFTRGRAAYMYTHTPNQLGFVGDTAYWDQTSRSGFTVTVNADGSNQTLNEDASQRKQTPSYFTSLFQTGGKSLKIKEVKYITYNNVMVANLTVESTQDRDVTLTTASPFAAEGADGATELTGRVNVKNNLTTIYPRFSANNQDGSNWIVSGGKLTSTLSLKANEPQTVKIQLGLIANELPDSTKEYEARYTGDLKDAAASYKDSVTTYNKWWVDNAPYVDTPEDNIDKTVVYRWWLSRFNMLDANMPGNTFQYPTSIEGVLGYNNQIVLTSGMFMMDTKWFRNPEYSYGTWLSAGDTAKKSKAGYYYYHDNPGDPANWNHSYTQYITRAGWDSYKVHGGPSTVAEELADQGAEDVQGLLASKSEPDNNDNQNNNDNSLIDWSWWSMTGNDADAVSFSEPGRSGQRMDRADGSANMWANANAAAQAYKAAGDTANAEKMQAIADKIQKEVTTELWDKSDNLLKHKWLNDGAFAKYKEINNYYPYSEGLMPTGNEDYNKALRLFEDSNEFPIFPFFTANQADKAALNFPGSNNFSIINAQPLLQVYSAGIRNYDAAKNGYITNEQFKKLLYWVAFAHYQGGDNNYPDQNEFWNEDNNNVGDVNGDGVINNLDKNLDAAQNGGKITYRSWIHHTQLGTTNWTMVEDVAGMVPREDNKIELNPIEIPGWNYFTVNNLRYHDQDVSIVWDKDGSHYGGPAGYSLYVGGKLAFTSDKLAHLIYDPAAGTVEVKDDSSAQVTVGAEAVKNVKAANQVTFNADQRVTDLFAKSGTNVDSASKSTTNVAKDADVTGTTYAEKDTNYPAKNAVDGKTVMESFWGTKGSENKTDTLNIKFKDGKQKIDDIRLYFYQSSSSQTISGYAEPANYKLEYQKDDGTWAPIADQVRTPNYAGANYNRIQFTPVETTTIRVTFTPQAGMAVGVKEIEAYNTGIKADGTSENQTPQVDAYVSSSTSSGAKLVGTVKDDGLPAEGDVTTTWSQVSGPEGGTAKFVDASAASTTVTFNKEGDYVLKLTASDGEKEGSKEITVHGIPSDGTVNVAPQSSASASYTNGYQPKDNAKKVIDGQVVYANTPNETWNNWGDSTGVEPWLQLKWAGKVPLKKAKVFFWTDGGGVPMVSSWKLQYADADGNWQDVKLADGQSYTVNRNEGNEVKFADAVETDKLRVVFPKGAIVGASEFEAYAIEPVSVDEVNRLVQTGSKADDLKLPSTVSAVYTDGSRRDLAVTWGKVTDAQLAADAVFDVKGTVAGALNGTVAHIAARSDTASQTVGNAQPVEQTVYQNAKSIDLPATVPVKFPNGYNDDRKVTWKDADIKAIDLTKVGDYEVAGTVDDGSSSAAAKLTVHVVADPNGSSTPEPEPEPLVGWIEGKATRTTISPDSEATWSPAEGKLNDGVVVDDTWPTTDDQNVNDKVWGSWGKAKDGMYAQYDFGQSVTIDQSRAQFWANFAETDDSKGGLEVPDAWKIQYLAEDGSWKDVEPTEDYTVVRNSPASRADTDAKGWSAVTFKPVTTKSLRLVLTPYTGSSTFGAAVAEWGVHGIDGTEPEPTPVDKTALESALDTANGLDASRYTAASWAEFQQIIDAAQAVYDDANATAEQVAEQVTKLEDGQKALVALATDVEKSTLQAAIDAAKAEAASGKYTDKSVEALNKAIEAAEGVLKVGEVGEVTQAAVQEASASLNKAVKALEEKPAAETVKKESLEASIEQAKKADKSKYTEEAWQALQSQIAAAQKVYDDKDAKQADVDAAQDALDKAFWATKVEQKPGSQQPGVTDTDKDDKDNKGDRVPPTGAAVSVVAAAAVLLTAAGVTILKRRQSGDHGSARHSA

SEQ ID NO: 10 - protein, 588 aa, unidentified
MTQQPQTYPKPAWLENARFYEVYPQSFADSNGDGIGDIPGITGKLDYIAALGCNALWINPCFDSPFKDAGYDVRDYTQVAPRYGTNDDLIALFEAAHRRGMHVLLDLVPGHTSEEHPWFQASSRPNPAERTIVASSDGPSRKENTGDPPTAPSGEGAVGEADWGRDQPTNVSNRYIWTDSWIAGGDGLPFIGGETERDGTYILNFFKCQPALNYGFAHPHQAWQKPALGPDALATCDAMIDVMRFWLARGADGFRVDMADSLVKHDDAGKPYTIRTWQYMFSKIRPEFPEAAFVAEWGRPNESMQAGFDMDFYLDWRWDGHPNGYNLLLRNTDTPMEHEGDSSYFNADSGASIAPFLAEYLPQLADCRAAGGHFNLITCNHDTLRTAQRLAERELKVAYGMLLTMPGCPFLYYGDEIGMRYRALPTKEGGYVRTGSRTPMQWASDAPNLGFSTAPADALYLPTDPADDAPTVTGEASRPHSLFNWIRSVLALRAAQPALAAGAGFSVVAAPADGRAFAYLRTAAPNDDSGDGTAAGPLLIAMNPGRHSETVGLPDTLSPDAVMLLGLGEPAVGDGQLTLSPQSFAVLG

SEQ ID NO: 11 - protein, 1238 aa, unidentified
MSFHVSAQSVRAVAGGLVAAATLLSGLALAPTAMAADSATADNAPSVAGHAYNELPYNNPDVTVTQIDNSALPSYMRNPIGQNEGIDTPNDLSQNYYSADASAQSYDGKLFVFTGHDEASPDYGSFNMKDWGVYVTDEDGLNQGKWTHYKTIAKADLFSWATGDGAYAGQVVADDNGTPSDTSDDWFYYYVPVKDKASEAAGQDPFAIGVAKSKSPLGPWKDAIGKPLLTTSQTQIETIDPAFFVDEDGTGYLHFGTFGTQLAIKMKKDATTGRTSYTEVETKADGTTPNLHTMKDADSNANGPKGFFEAAWVFRKGDTYYNVYDGGKPGSGTATCVESNYQACIQYSTSDSPLGPWKYQGVIVPSGSATTMHPSVLQFGDKWYVTYHTGDKEGGTDFRRAVCIDEVDWTADGQMVSTAHPTKAEKTQPSTNVAPYAKVSATFTETPAWKGSVNDGRVLQTAVVPPNHWTNYRSIPQSQSGDSLVYQWDGTVRVNSSKVWFDVDSNALRAPASWKIQYLDADGTWKDVTNPSAYTTTTGKANPNAVTFDAVTTTALKLDMTGQAVDGGYASVAVAEWEVGSDSSESPAITAPKSVTTATGTAPTLPATVDVKYGNPTVASPVIWRPVDASSYAKVGSFTAYGVVAGVPGEASEQGNVSVNVTVQDGYQPAADTTKPTVTVAVTANAGNSEWLTTAPFATVQATDDTAPIAKLEISADQGKSWTTIAANANAAIATLSQQGDVEVWARATDQAGNVSDVAKAGGKVDSAAPTVTAAADKEERTLTLTADDGTGSGVASIEYRIGTDGQWATYSKPIAAPSASRATVYYRATDKAGNVSASAKTDIPSDTSVPLTGYIEGDATATDVDGKASGWVKGAAALNDGKIIPDITIANEDVWGTWPNTGEMRLDYEWDREVTIDSSRVQFTSDDGGLGIPASWELQYWDALANNGAGNFVDIPDATYTVTANSPSAGWATGDAKGWSDGTWNTPVKTTKLRMVITSGSASPAVAEWQVHAIDDSTPEPPEPTPIDKTELKQALADSPKADDASKYTETSWAEYAAVLDSAQQVYKAEDATEAAVVDAATQLKQAAKKLVLVATVQDRAALSAQLDAAAAVDRTKWTDESLAVLDSAVATANALTSDGQAAQSDVQAATEAISDAIAGLVEKSTTKPGQGGDKPGSGTDKPNQGNDSNQNKGDADSGKHKKIPDTGAAVLGVGILAVVLAVAGVIILKRRKSGTC

SEQ ID NO: 12 - protein, 673 aa, unidentified
VESVEKTEVAKKAAKTASTRAAKTATTSTATKTTATKRTTSRTARTTATKATTAVRSARTSKAAATKSATAKRPASSAVFNARLAAHQDELEQLFMSLYNDHDAFDALVASMAAAYADRPADLKRLDKTREQDPGWYKRGDMFGMTMYTDLFAGDLKKLADKIPYLKEQKLTYLHLMPLLDMPHPNNDGGYAVQDFDTVDPKLGTNEDLAALAKKLRRAGISLCIDFVMNHTASTHRWAKAAEAGDPEYQDYYFCYPDRTIPDRYDRDVPQVFPNTAPGNFTWNEKMNKWVMTQFYPFQWDLNYRNPKVLVAMMGSVMRLANLGVEVFRIDAVPYIWKQLGTNCRNLPQVHTIVRMLRIILECVCPAVVLKGEVVMAPKELAAYFGTPEAPECHMLYNVSIMVNLWSALASRDVRLLKNQIDKLNSLPENCWFVNYLRCHDDIGWGLDEDVERYLDIDPLKHKEFLYHFYEGATPGSWSMGELYNYDEATRDARSCGTTASLCGIEKAIITHDKPLLETSVDRDLMMHHAMSFLRGFPMLNCGDEIAQLNGWDYKEDPDRVEDSRNLHRSKFDWTKAALRKKSGTLQNQLWKGMEDLRAMRNDPCFAPDAWVSTWDTFNDSVLAVIRQHEGKTLVGLFNFSPDPQHAHLNACNGILNAMDADLQPYESQLIHL

SEQ ID NO: 13 - protein, 791 aa, unidentified
ISRRAVLAGAAAIGGTIAVASALGTGVTTVQAAPSVAGTAGATGANGAGGVAVPAAQRTTKSAKTITDAPALPGWKVAPFPLRNVAITSNSVFDRAKEGMLDYARNYPVDRWLVCFRAQANLLPKDNTTQPSGGWENFPSGSLDKAVEQQWGDAEYTRGQNKNGADGLLRGHFAGHALHMLSQAYAETGEEAILNKINEFVSGLKECRDSLREMKYNGKARYSHPGFLAAYGEWQFKALEEYAPYGEIWAPWYTEHKILAGLIAAYEFAGNADALDLAEGIGHWTYARLSKCTKTQLQKMWDIYIGGEYGGMNDSLVDLYNVSKDKDRSEFLKASAFFDTDKLIDNCGAGVDILNNLHANQHIPQFVGYAKDAAMGDADIDADARARYLKAVEGYWGMIVPGRMYAHGGTGEGEMWGPAHTVAGDIGKRNAESCAAYNMLKVARYLFFIEQKPAYMDYYERTILNHILGGKSRDLDSGTALTPGNCYMYPVNPATQKEYGDGNIGTCCGGTALESHSKYQDSIYFHSTDNKELYVNLFTASTLDWTDTGLKLAQETNYPEEETSTISITAAPKSAVTFRIRIPAWSKGAKIEVNGKAIDGVTAGEYATVAGSWKVGDKIVVTIPLQLRTESTDDRKDIQTLFYGPTVLNALNPSTKFIQRGFYERNGLDGTIKLGVQKKEGTKNYFIIDGDEFEPAYNGDNTPYHMYFQRKDEYVGFAGKLTDVLNPKRPAAQDRSESTLMDDIWAGAPFKDRAAFIKKVQEVTKTYQDEGLLSSRDRQVVLLASARAKMS

SEQ ID NO: 14 - protein, 449 aa, unidentified
MVLFMANPQRPKMYEKFVHDTPEWFKGAGLGIFAHWGSYSVPAWAEPIGALGTFDDPVYWNTHCPYAEWYWNTMSIKGSPAAEHQKEVYGDMPYEDFIDMWKAEAFDPADMADLFARAGARYFVPTTKHHEGITLWKAPDNDGWNTVDRGPHRDLVKEFADAMRDKGLKFGVYYSSGLDWHKEPNMPILGDGEYGPQSEDYARYMYSHVMDLIDEYQPSILWGDIDVPKISEEDNDFSVARLFEHYYDVVPDGVVNDRWGLTHWDFRTVEYEQGKELMGKGMWEMTRGIGYSFGYNQMEDADSYMTGPEAVKLLADVVSMGGNLLLDIGPDAAGRIPELQRQCLEGMADWMDVNSPSIHDVEPVPEASPSGEGDGEPWVRWTGDGKSVYAVVDAAGRVPLRIDAGAVDVDSATILGGGNVVVEADGDMLTVEIPATDVAGPQVVRFARH

SEQ ID NO: 15 - protein, 657 aa, unidentified
MTQYHVSAQAAKTNGAGSEADPFLTIGQAAAIARAGDEVIVHAGTYRESVDPRFGGESATNRIVYRAAAGEPRPVITGSERIDTWQPEGDGMWKAVIPNTFFNGYNPYVETVFGDWTVYPDPKAEVRHLGDVYLNGKSFYEVVSLDKVRNPQRWDTGRDAATDSIVPLIDPDATVNVWCCAVDDEATTIWANFHEADPNAELTEINVRETCFYPSRPFVNYITVSGFEMAQAACPYTPPTADQVGLVGPHWSRGWVIENNRIHDAKCSAISLGKEISTGDNESTRTHRKSGYQYQKEAVYKALHAGWEKGVVGGHVVRGNVIYDCGQNGVVGHMGCAFSLIEGNHIHRIGTKREFFGWEVAAIKMHAAVDTVVRGNRVHDSVLGMWLDWQAQGTVVDSNVFYRNTRDVMTEVTHGPITFMNNVFASEFSFDDYAQGAAFVNNLFAGRIRHTAVLDRSTPYHFPHATDVAGEAFVYGGDDRYINNLFLAVGDSAKPLRTAAGAAGMAEAGTAFFDGYPRSLEEYEQLIEEAGLGDEELYRSVKQPVLLASNAYVSGAKAASGEAEAVVSDDGSSLALRETDDELWMTVSLPEPIRSATGPVISTADLGQPRIVEEYFENPDGSPIVVDRDITGAARGACSARGPLAAYDDGEVLIWSK

SEQ ID NO: 16 - protein, 539 aa, unidentified
MKISNPVLPGFDADPSMIRVGDTYYIANSTFDWWPGVRLHESRDMVHWNLLPGPLNRVSQLDMKGNPSGGGIWAPDLSYADGKFWLIYTDVKIVNGAFKDCANYLVTADDIRGPWSDPIRVNGVGFDASLFHDTDGRKYLVQQTWDFREYHHPFDGITLTEFDVKTMQLKPETARTIWRGTDVKLVEGPHLYAINGWYYLFCAEGGTQYEHQESVARSRTLGELSFESMPSNPFLSNFDTPGTYLQKQGHGALVDTPSGEWYYASLCGRPWRHENEPAHGVRGWCTLGRETSIQKVEWDGDGWPYVVGGHGGERYVEAPADAIATEAPTNHSQHDEFESNKLDLNWNTLRVPFTETMGRVGGGSLTLRGQGSLANLFELSLVGRRWQAFDFNAETKVTFDPSNYMQMAGLTNFYNHLCWSWAFITWDEKRGQRVIEVAQNDFDTYTSFLKDTAIAIPDDIESVWLRTKVRTRTYTYEYSFDGKTWHEIPVTLDAAVLSDDHVAQRYGGFFTGAFVGLVAVDLSGYGKEAIFSHFDYMEI

SEQ ID NO: 17 - protein, 588 aa, unidentified
MCDRKDQSTFKARACVITNPVLRGMYPDPSWIWDTTREGIVLVNSSFELVPGLPIHTTRDLVTWTRTANAVDEAMSHRLLIDGVEDSGGLYAPTIRRIGGRYVIVCTVARVDEVKALAAGCSQKDIDNCRDSQGNFVIEADELTGPWRGPYWVAGAEGIDPDIFEDGDGTVWWTQTRPALHPQWDGQTEIWTQPIAPGTWTLQDHRTVIWRGYGMDAVWAEGPHLYRVGDYLYLMTAEGGTSFEHSEMIMRTHAPRGFAAALAGFEKNLAAQDIWIEPARDGERSVVGRDDRLFEACKRNPILTHRHLGAGEPVQCVGHADLLLHPTVGWLLACLGVRETPGEEPGETFSYCGRETFVAPVVWQHNPVSWKLDGGEPNKRIDIVDPGWPVVAPGLGRLPEKLVVDVDEDGLPCAVRGVDSARCDCAAGDNAACSAELIQLDDTRLVGVRGRDDYRFLRVDGQEYAAFTRPEYTMMMHQDSTHVVMVMAEAESGQLTAEITDRGDKRCIALGALGANEWFGLRLHGNRLEFLAGQPAPDSIDEGTMPDIALIAHADVTYARIMADCDARFLSTEWAGGFVGCLIGVKRR

SEQ ID NO: 18 - protein, 428 aa, unidentified
ITEVALYDIDQSRLLVIEAILRELAEGFEDVPKLTVSTKLEPVIEGADFIFAAIREGGLDGRCCDEHVALDLNVLGQETTGPGGIAYAIRTVPVMVHIAEKVKRLAPDAYFVNFTNPAGIITEAMQTVLGDRVVGICDTPSGLGRRVAGVLGADYRRAQMDYVGLNHLGWMRRVVVDGKDLLPDLLADDERLNKMEEAHIFDKDWIRELGALPNEYLYYYYDNKDAVRRILESPQTRGDFLKTTQTKFYHDAAHVAQKGDDVATLWWRTVKERDASYMAEALGGVQGAPATERDLDLDPAQLGYAGVALGVMMAISRNDRQIMILNVRNRGTVVDLPDGAVVEVPTMVDANGMHPLSLYAQPDLHQIGLMQSVKSVERHAIAAGLTGSNKEALLAFAEHPLVGSVPIARQLLEGYKTRIPGVAAALSR

SEQ ID NO: 19 - protein, 1278 aa, unidentified
MKINNKGKGALIAAITAAATLLSCGLAAASASAAGVDYLPTIGQVPTYTKFQPTADPGKNASDYFQPYWYAKNANDNGGTHIQAHGGQVVKVGDAYYWYGEDRSNGYDNSPGVHAYMSTDLYNWTDLGVALRAVTSKSQLTDKSNADYAYFDKAYNLTKSDGSVDAAKADAIFPYLNTNPDQDGDGAVDSVQGIFERPKIIYNKKNKQYVLWWHSDGSTTPGGSNYARALAGVAVSDNPAGPFTMVGAYRLPNQNNWKEAAGNPSWGENGDSRDMTVFVDPKDDSAYVLYSSEANATLYIAKLNDDYTNVVKTTNVDQSEGQKQYSADGQYPYILADATTDAPVRGEDFQIVKQNGSLEAPAVFQYDGRYNIIASGATGWAPNKQTYYTADSMLGSWTRGVEKDDVNENTWYNNMPEGADGLLSVGDIRGTTFGSQSASVLAVDQEKGHFIYLGDRWDSGKADSTYVWLPLTIGENGTIEMHNPAQEGEPDGWDLSYWGNHGSAKGKLVNWTVETGDDLPKTVNTGGTVTLPDTVNVKEGDDTIATKVTWNVEGGTAVSKSTKAAGNTYAFNVPGTYTITGTLAESSNFNPGRTFRRTIDVSCSNPISGSWKEAHWKGGSACQVSASGGAYDFTITDNANRGVWTDRNEGSAVYQPDALDVNETLATTVKPLDLGGNGDPRAGLVVRNGLTGANGGKGYATLLASPSGVYMQYDSNADGYIDKETSHVGTGFGDQVQLKLERTSTDTLKGYWRASANDEWQDVATVTLTGADVTGLDAGAFATSNSNAGAFTVAFNGTAFGSQTAAVESIAAKGPEATIAKRQTLAHKDVTVTATLTNGKTRVLEPDEYTLEGFDTTKLGEQTVTVRLVTDSSVTATLTVTVESNLARLFCSSAAASKYEPASSWASASTADLTCDNNLSTNWSNWGTGDTSPWLSYTFDKAYQLGKLSVAVDKAKGEAAPKSFTVSYLAEDNATWTDATLPAVTVNGAAGAVTEADVSALPATKGIRLNFTYADGNDYAKIAEVRIAEGEATPKPQPSSNANLADLTVDGKTVDGFSADITEYAGALAGDAASYPTVEATAADAKATVQVEQASTENSGVATVTVTAEDGTAETYTVTFGELPQLAELAVEVTKDSYQVGDKFNAADVKVSAIYKVGDTETLRKLIDPTDGDLKFTGFDSATAGTKTITVSYRGVNATFEVTVTATEVTPGPGEQKPGDTNNPGNTAKPGNTATNKPAANGAAPLSNTGVAVAAIAVVVVVLAAAAGALLVIRKRRA

SEQ ID NO: 20 - protein, 1063 aa, unidentified
MKHWKKMAASLIAISTMVAVVPTTYAMESEDSQPQTTDTATVQTAKAAEPTLLASWDFTGKNGTTNSAIADSTGKYNLTLKDNAKIEQYGDRSNNEALSLRGDGQYAQIDDQLFKDAGDSFTLEFASKTRHDDSGKFFSFIVGKDGSNDANTTDQSNANKYLMFYNSKTAIKSVISNNNWGNEQGSKVTVSGNDNSWADYKIVVDGTNLAVFRNNALIIFKANTGIKMSDLGATTAYIGKSFYSVDEYWNGAMDDIKVYRGADLTMPTAVAISGTGVVNNKLTLIEKDSTKLTATVTPDDAVSKNVTWSSSDESVAKVAADGTVTGVKAGTATITATTELGGVKAELPVTVEPMNAQNAAAADLDAAIAALKVPAAENLPLVAKGTKNGSAITWKSSDEKLITGTDAKYEAPKNGMADPYKGAGVVTRPAYGDGDSKPVTLTATASYNGGEVVTKTVEVTVKEQTRSVPDGAYAAVTFLSDEATTNGKIGEALYESATDGNNFFSFSEINSGNPVIVSNTDTKGLRDPYVLKSKDGDKFYMIATDLKVSSQGWGQNQQYGSLKLEAWESTDMVNWTRTNAEDGDTGIKVNADNMGMTWAPEAFWDDALNSYVVFWSSREYTDDTRATAVKSSKTNGAYNILVMATTRDFKTFTPAEKWQDTQYSRIDSTVFKIGDMYYRLTKNEESGKAGDYITNGKSTFLERSQCLTCTTAAADPNADTTKTWQLVDQNILPFEGPESIKLNAGDPNQNEAGDAMIIMADSGGYKPYMTSESAIAASSWTNRLSSTDGWGTEKQWGPNVTGKVTPTNMPTPTRHGAFVNVSAAIAENMHKWNTSTPTTLPAVDSATKAEYNAETRELTATVTAADKGTLAGSVKFSAGDWSKTVKLDAEGKATVTLPVTISGTVAVAYDGYTDGLVNPSSTEVAGIEQGKVDLAELNKQIAAAEALKESDYTADSWAALAKALQAAKAALASSDQAVVDKAAADLSAAIAALQKAETPTKPDTGDGDNKGDNAGDKTDNKTDDTKSDGKLPATGAAVYGVSDAMVLLAAAGTALSIWRKRRA

SEQ ID NO: 21 - protein, 1513 aa, unidentified
MRVLSKSLAAMVAAATLVGGGAFAVAGTAYAADNDAITVTPNPWYANSFDGWGTSLAWFANATGSLGEESAITTNLGDDASKAKAVEYGKQLREQFYQSIFGDEGLDLNMARYNVGGGNASDVAYGYPFMRQGAAVPGTWKDDATGSGTYGNGVTTKQADKDKLAAAFDPTDDNQYDFSKSAAQDWWIERGATGDNPDITDVEAFANSAPWFLTNSGYATGGYNSGSNNLANPEKFAQYMAKNVEHLESLGANVDTVEPFNESETSYWGTPGDMASKYTDESDDNTKLINNYWDKYYSDKDRSVTPYSNALKKPQEGMHVSNAQQQQTITALAEALKDNDDTIIAATDATNSADFVKSYNQYPQAIKDLIGQYNVHAYSDSNQMQSRDIAQADGKKLSMSEVDGSWQSGSYNPYGFDNALGMMSKISSNVTRLQSKDFTFWQVVEDLYNMQMGSNVNPAGENTNWGTVLIDFDCTVAGMDGKLYSERRVNNNGGTTDGLEPCTVIANAKYNGVKAITHFIHAGDKVIANNDEDNNMTATSDDGKTQTVIHRNSGTSDQTFVIDLSKYGEIADNAYGELYLTTETSAEDKNAGVDSATPEVFAKTSNVKQAEGSVMIDKAAKTATVTVPARSIASIQLTGVTGYAKDAAVETGDTYQLVGKQSGKAVADTTSGDSALSLANVASDAENAKKQTWTFTQIEQPADSERPDLKAYVITNAEGKVLVSKDGTNALSNETVEAAKSDPAAKWILNTSDGSTYQLLNAATKTNLDVDNSGTTVGTKVGLWQSPSGTSPSANQTWTLRNVTPTSQKTVNVQTAVNEKAALPTEVTLYYTWGEGKATVANWDTSKVDVAKEGTYEATATATDVYGNEFNVAATVYVGALTVSDPVSATVLAGTSASEAKAALEAAPVYLHVKASPAFEGDAAKVTWNFDGLDTKLADAKAGDNIAVTGTYQLDDATTIALKGAIYVTAATPENVADTASSLTVTNQQTEYSKGDQWKKLTDGDTSAEAWVTWNSAGDYSASPTATIDFGSECELSRVTITYGDKAPASAKAEYTTDGETWMQFGSDVKPAAGQTVTFKADKGTVNATKVRIVNTVNNDYMNATEIQAFVTPVQGAAKNIAAASGTNFSVNFQEGASASKAIDGDTTSKGWSTWASTASTVDPVATFTFDEAQTITEVKTFFYYDGRASWPKSQTLEYQDEAGEWHEVGTKDGWKTQAGDAGSGSDGITAADTPTVDFVLDTPVKAKAIRLTNTLQDTKVYINVAEIQVFAQDNKVITPQPASDATLGDLRLDGETIKGFAADKTDYTVDLPFGAEANPVLQAFAADNAATVKVTGDAVENGKLGGKATITVTSADESETKTYTVTFKAFTLASLKVTGPTKIEYAIGDKLDTAGLKVTAVYQSGDKTKEVPVALDDPQLAIGSFDSTTAGKKAITVSYRGVTATFNVTVKANAVAPGPEEQKPGSTNKPGATGNGNKNTVANTGSNVAAIAGAVALLAAAAGALFMLRKRA

SEQ ID NO: 22 - protein, 1099 aa, unidentified
LAAVLSMATLAALAAPASATDTMPWGGQYADTSHPEQAAGANQPYQHGYTGLDILNWNPAVDKDSEYLRSRVPLQTRIAANAATQKDPNLPADTEMFNLAGDYGNAFFESFHDNNVFSQYLFNYWQYTDYYGTWHGQPTANVPKSLYDEKAQSDWTQKWFEFGTLNLPNAAYTNAAHKNGAKSIATIFYSGNDRGEQTYKDLLQGKRADGTYPVADKLVEIAKYYGFDGYFVNQESNVNSDDVPAYRDFMKQIVNQGIYIQWYDSATYPGGNIGYQNRFNEKNSPWVQDGAEGKISDSIFLNYWFSGNMLKDSADHAKSLGIDPKYAVFAGIEAGQQKFGSIGSNADYMNVNLDADGKPYVSLAALGTDFVSHELGDDKKVYPKYQNQVFDRERRLWTGSSTGEKGTTDISDPYIDDGTSSDGWKGFASQIAERSVIGGPVFSTAFNTGHGLEWRDNGEQTSNQQWGNINLQDILPTWQWWIDADSDPLQADFDYGKDYETAPRFNYTKVGGYEGGDSLVLSGKLSSDNTVRLYKTDLSVTAGSKIELTYNKLNSDDSKLQLGLIFADDTNTVVPVDVADGAASNGWKTATVDLSQYAGKKIATLGLIVKAGANPIDNYQVNIGKITVTDGAAYTPAAPTGFKVERAYADSDELTVKWDLADYATTKNYLLYLDNTFLGGRYDDTLYVKHLPAKTGTLKLYAVGADGSRSMAAEAPLNEAAAVSSVKVEPGKDGNVKVSWTNPQVSGEKTVTVKSDTGSWRYAGQPYSQTVKVAADKSDVTIPNAPVDGSRYVVTVDNAGGATTTAAGAFADATIEPYPACSVTWNGDNVSLIRPETQDWRYLYMTEKWTDENGKKQEKQLGAKYTYSQNTPPITGIIRGRTTPESYTKSIPDGHELWVQVEDYSGNKTDPVKIPTSDELAKCTVTDPTQPDAKKSTLTAVDGSAVADGKDTRTVQATVKDSFGNPLTNAEVAFTLPEGVTAVDGNATVKTDAAGVASLNVVSTKVGKYTVKAALGDKSIGEGVTIEFTKVAVTPNKPGDQNKPGDTKPNKPSQNDNKNNDKNGASLSATGSSVIAIVAAMVVLLGAGAVVLRARSKTE

SEQ ID NO: 23 - protein, 660 aa, unidentified
LIMNVTITSPFWKRRRDQIVESVIPYQWGVMNDEIDTTVPDDPAGNQLADSKSHAVANLKVAAGELDDEFHGMVFQDSDVYKWLEEAAYALAYHPDPELKALCDRTVDLIARAQQPDGYLDTPYQIKSGVWADRPRFSLIQQSHEMYVMGHYIEAAVAYHQVTGNEQALEVAKKMADCLDANFGPEEGKIHGADGHPEIELALAKLYEETGEKRYLTLSQYLIDVRGQDPQFYTKQLKALNGDNIFPDLGFYKPTYFQAAEPVRDQQTADGHAVRVGYLCTGVAHVGRLLGDRGLIDTAKRFWKNIVTRRMYVTGAIGSTHVGESFTYDYDLPNDTMYGETCASVAMSMFAQQMLDLEPKGEYADVLEKELFNGSIAGISLDGKQYYYVNALETTPDGLDNPDRHHVLSHRVDWFGCACCPANIARLIASVDRYIYTERDGGKTVLSHQFIANTAEFASGLTVEQRSNFPWDGHVEYTVSLPASATDSSVRFGLRIPGWSRGSYTLTVNGKPAVGSLEDGFVYLVVNAGDTLEIALELDMSVKFVRANSRVRSDAGQVAVMRGPLVYCAEQVDNPGDLWNYRLADGVTGADAAVAFQADLLGGVDTVDLPAVREHADEDDAPLYVDADEPRAGEPATLRLVPYYSWANREIGEMRVFQRR

SEQ ID NO: 24 - protein, 549 aa, unidentified
LAMNGNAGIAQKVQKVTGSNPVIHSDFPDPDIIRVDDAYYMASTTMFLMPGCDVLRSYDLIHWEFLTHAYAELDDTPAENLEERGRNAYGCGMWAPSLRWHDGLFSIVFTSNDTQKTYVLEACNPAGPWAKRVIEGFYYDNSVLYDDDGRVYIVHGNTQLHLTELEADLSRPKAGGLNRIIAEDEPDADLGYEGSHIYKHDGRYYVWTCHYRSGCRKSEVCLMADALDGEFIAREVLDDDLGFHDLGDAQGGMVDTPDGQWYAFMMHDRGALGRVPTIMPMRFGEDGFPILGNDGRVPTEVSVPTAYADYPYRPLNGDGFCCATDSDKSAEDVADIVKSDKLAPFWQFNHNPHNQFWSVNEPTGTFTLHGGKIAANLNYAQNTLTQRTVGPRCMAEVTVGASQLQDGDFAGLAAFQGYYRYIAIRRAERGYALSVRIKPKDDDSIWGDGNYDASAIEVARTNLCGDTVRLRAIYDFTDKRDKVAFEYSDLAASAPHWIRLGNTYDLYYTLDYFIGCRIGLFLLPTEKRGGTAAFSEFKFFGPEETPELS

SEQ ID NO: 25 - protein, 1964 aa, unidentified
MKKKKTISAALATALALTCMGSGGGTAFAVPLSDAELQTLASQIQQINNTSDSATASETPAAQADATEGWTIDSNIAKGGEILETVDGWLHFKSTEAHGNAASNPSSSNDWPAVAVWGKDYDFSKAGSFHATIKSPQEGKANRFGFYLGYNDPGSGLFIGYDSGGWFWQTYTGGGNGGWYGGNRIAAPSANDEHDIQVSWTDAKVATLTVDGQKAFDVDYSAMTNLSNKLAIKAGSWKSLNQVTDVYIKDFPEVVEAAKHAVSGKVVDAEGAAIEGATVRLDKTKVKTGADGTFSFADIEEGEHTLSIAKEGYEDVSQQVTVSGADLAIDPITLNKTVQVASETLKTKKMEVQIKKNFPSVLQYTMTDGKVMYGQSKDVRTVEINGTNIELGDDDVTFKKVSDTEATYTLKVKDEAKKIDAVITVQITVKANQLHFNVTDIKNNLSDGIPEGNGVEKNAIQTLSFPNQSLISVRSSQDGAQFTGTTMSSNTQQPGDENFAVTEDTNYTDRDYTYGFISGAGLSAGLWSNSEHDGRAAYAGVRGGSQNTRVYATTQQTGDATSLGLASAPWYYHRTVTDSKGKKYTVAETAMPQMAVAIAGDENEDGAVNWQDGAIAYRDIMNNPYKSEEVPELVAWRIAMNFGSQAQNPFLTTLDNVKKVALNTDGLGQSVLLKGYGNEGHDSGHPDYGDIGQRLGGADDMNTLMEEGSEYGARFGVHVNASEMYPEAKAFSEDMVRRNSAGGLSYGWNWLDQGVGIDGIYDLASGSRVSRFADLSKEVGDNMDFIYLDVWGNLTSSGSEDSWETRKMSKMINDNGWRMTTEWGSGNEYDSTFQHWAADLTYGGSTLKGENSEVMRFLRNHQKDSWVGDYPAYGGAANAPLLGGYNMKDFEGWQGRNDYAAYIENLFTHDVSTKFIQHFKVTRWVNNPLLTEANGNGNAVIDPNTNNGNEQITLKDGNGNVVVLSRGSNDSSSAAYRQRTITLNGVTVASGVVSAGDGSATGDESYLLPWMWDSSTGKLVKDSEQKLYHWNTKGGTTTWTLPDSWKNLSSVKVYQLTSQGKTNEQTVAVSGGKVTLTADAETPYVVYKGEAKQIQVNWSEGMHVVDAGFNGGSNTLTDNWTVAGTGKAEVEGDNNAMLRLTGKVDVSQRLTDLKAGQKYALYVGVDNRSTGDASVTVTSGGKVLATNSTGKSIAKNYIKAYGHNTNSNTENGSSYFQNMYVFFTAPENGDATVTLSHKSTDEAHTYFDDVRIVENKYSGITLDENGELKSLTNGFENNAQGIWPFVVSGSEGVEDNRIHLSELHAPYTQAGWDVKKMDDVLDGTWSVKVNGLTQKGTLVYQTIPQNVKFEPGAKYKVSFDYQSGSDDTYAIAVGQGEYSAGSVKLTNLKKALGETGTAEFELTGGVNGDSWFGIYSTATAPDLQGSTGSAQDFGGYKDFVLDNLKIERVDSQNRTKAEAQAKLKEIRGEYDSKRSEFSEDAWNTYQANLVKARVLIDKDGATAEDYTKAYDILVALDEYMKSAPGNEGSDAYDVAADGSDQLGGYTVEVGSEEPSAGLPSEGPADLAQDGNGSTHWHTSWSANAVGDGTAWYQFNLKEPTTINGLRYLPRSGGMNANGKIKGYQITLTLADGTTKDVVTDAEFSTSTMWQKASFDAVENVTAVRLTVLSSTGQSDSQANKFASAAELRLTTNREVEEETVAPDKTDLNDTIAQANGLKESDYTAESWAALVKAREAAQTVADDEKASAYDVALALANLESAIAGLEQAGEEPGPGPVEVNKTDLQAAVNKADKLEKADYTAGSWDVFAKALKDAKQVLGNENATQQQVDDALKTLQDAISKLEAATKPKPEPGVVDKAALNATINKAAAINLGLYTDDSANALRAALKKAREVSADNDATQKQVDAAREALEKAIAALVNRPAAKGDGNVVSNTGSNVATIALAGLLLAGAGAAIAYRRNREQL

SEQ ID NO: 26 - protein, 469 aa, unidentified
MTDLPQSLPQAWRPPMGWNSWDSYGTTLTEDELLANARFMAEHLKNAGWDTLVIDIDWYDPTARAHGYNDNAPLILDEYGRQLPDPVRFPSAAGGKGFGPLAAAVHELGLKLGMHMMRGIPRIAVDKNLPVYGTNYTAKDVADLDHVCKWNPDNYGLNQSHPGAQAWYDAQLDLFASWGLDFLKVDDMQTPFHSDEIAAYHRAIAKAEAKYGRSIDLSLSPGGWVATSYVDFLRENAQMWRISDDLWDRWEDIYQQFARLARWAPFQTTGHWADADMVPFGHIGLRAERGDDRQSRLTLDEQKTLLALWCMGRSPLMVGGDLPTSNSDAIALLQNPALREVLAGSTNNRETVRERIFGKWWDESTYRGEFIVWSADAADWADGTRSAHHGGHYAALFWTGSDTYEIGRNIQLQSIVGLDARNDDWTLADLYADAPGEPADVRLEGVGADRVITGTIPPHGVLWVALDRR

SEQ ID NO: 27 - protein, 813 aa, unidentified
VTAASADEPVELVVNGGFEDDLNGWKSGTVWNSSASSIAVTADDVHGGSGALSVTDRTSSDAGAIQSLDGKVEKGQTYTGSMWVKATEDGEFNITVCSGNGSGCDQIATGTAKAGEWTQISGTGTLGGSGDFTSPSLVIENKYGTSNADFIVDDISVTGSDSGSSFVPPTTGTATAAKAFGDYSNPIIDYWYGADPWAMEYNGRVYIYTTGDGTSVNADGSLNYDYEYDSTGQIKDNSFAQVKTINVLSSDDMVNWRNEGYIRVAGEQGVATWASNSWAPAVAHKTINGKEKFFLYFANGGSGIGVLTSDSPVGPWKDETGELLIKGGTPESAGVVWLFDPAVFVDDDGQGYLYYGGGVPTGEEEHPNTARVIKLSDDMLHTEGEAQAIDAPAMFEDSGIAKIGGTYYYSYCTNFSHDNVIDGNTVGYGNIAYMTSDNPMGPFTYQGEILKNPSTYFGVGGNNHHAMVQLGDQWYMTYHAQTVAKELMNGGNLDAAHGYRNTHLDPITVNEDGSIADIAMTYEGLSSVKNLDAYQDGGIPASTIAWDSGIQNAYDLTSGVRVVDMTTDNSEGQKLSNINNGEWTSLANVDFGNNGASSLTVTAAGKAGGILEIRLDDPDSEPVASISIPAGDGSEYADYTTTLEGVTGVHHVVFSFKSDGGANAQDELYDITTYTFTESQDETPSTPVDKSDLQSAVNENSGLNASDYTEESWKVFADALQKAQAVLQDESATEQQVQDALAALNSARAGLKLADDSTDGNADGSNDNASDAVDDTDGVDLAQTGSSVLPGAVIVACLALTGVIVARVRREMR

SEQ ID NO: 28 - protein, 1240 aa, unidentified
IGKRLLGAMLAVPMALAGMGIGATTAVAADTVPTNNLIAAYDFTTKPSDGKTVANSAPNATLGAAEVQNSADSLWADDALTLSGGAKTGTGDWVKLPSNLLSGKDAATVQLEVKADSSMLNAFHFLWNIGNDSSDTEYFFATLNCGSSRNPLVGLKSGGTETLVQSSSCVAKADQWLSVTATIDGTAAKLYIDGTQVASGTVPAKLSSVKDQSLNTIGRSPWPDNLFKGAVSNFRVYDAALTADQVAAISTADASIHAGELTGSVLNGIIIPTTVDDPFISLPTANGVTWASSDSSVIATDGTVNQPAKGEAAKTVTLTAAVTVRGQTATKEFTVTVNPTTKTAAEQLKEVAAGYVIPSVVRSGDALPAAVNGTTVTVTSTKDVAVEDGKITIDGDEATTGTITVEFSKNGLAGIEPITKAFTVKVLPAAKSATIAAYDRNATGENEANNGDVAYSMHLALQNADGSYTPYNENYGIFFARSPKAQKLNENLDGNDYRSLKDPSLFRMADGTYGVISVRTNRGTATGDSTAKSSVLIATSEDLLTYSEQENSGSIVDLGETNGVNAPYAVYDTASKQYVVGWADDNGVAKYTTFDSLKGSTSKHGSVLYGSVAKSGVLAADGVQGIANFRSGATIAVDEATVKALNTRYGRSENTGTSNLTDITVKKGASIDEVTAQLPKNVDLTYSDGSTGSLPISSWNTEGIDLTKVGDYTVTGTVKQTEYQIPFAEDRADPSVYKWQWTHEADGKEVTETKFLMIASNDIQGDVTWQHGSPHMPFRMADTISGLADEPGNPNALIQSNGYNNKEVSLLKAGDKDSEGNAIMHSFWAPEIHEIDGRLTILFMAGYGNTWSNGKSVYMQLKQDADGHDLDPADPDNWTVPTPIYRNDASLLNGNKQLAATASGGVGMSLDMTYFQDADGRSYYAWQQLGATYIATMDPKDPAHVTSSPVRIVTPEYAWNAAIAEGPNVTLRDGKLYLMFSGSGVGKTYTTGLAVADASGTDLTDPASWTVLNYPIQKSGPFNGEMQLGTGHGMWSEDEDGNQIYVFHAYATKNLGSVNATGRDMFVRRVHWAADGMPVFDMSSSEELASKTVSVTVKVVDDTVTVDKTDLSKALASAKQLRESDYTAASWKAFAAALASAEKVYADENASQKDVDDATAALNKAQAALVKVDGSDSGDGSSDSTKPGDGSSIDAGATGDKTGNKLGLSKTGAAVLGLSGVAVALAAAGIALTLQRKRQA

SEQ ID NO: 29 - protein, 782 aa, unidentified
MKLTFDGISSHWEEGIPFGNGRMGAVLCSEPDADVLYLNDDTLWSGYPHAETSPLTPEIVAKARQASSRGDYVAATRIIQDATQREKDEQIYEPFGTACIRYSSEAGERKHVKRSLDLARALAGESFRLGAADVHVDAWCSAPDDLLVYEMSSSAPVDASVSVSGTFLKRTRVSSGSDSDARQATLVVMGQMPGLNVGSLAHVTDNPWEDERDGIGMAYAGAFSLTVTGGEITVIDDVLQCSGVTGLSLRFRSLSGFKGSAEQPERDMTVLADRLGETIAAWPSDSRAMLDRHVADYRRFFDRVGVRLGPAHDDDEEVPFAEILRSKEDTPHRLETLSEAMFDFGRYLLISSSRPHTQPSNLQGIWNHKDFPNWYSAYTTNINIEMNYWMTGPCALKELIEPLVAMNRELLEPGHEAAGAILGCGGSAVFHNVDIWRRALPANGEPTWAFWPFGQAWMCRNLFDEYLFNQDESYLASIWPIMRDSARFCMDFLSDTEHGLAPAPATSPENYFVVDGETIAVAHTSENTTAIVRNLLDDLIHAAQTMPDLDDGDKALVREAESTRAKLAAVRVGSDGRILEWNDELVEADPHHRHLSHLYELHPGAGITANTPRLEEAARKSLEVRGDDGSGWSIVWRMIMWARLRDAEHAERIIGMFLRPVEADAETDLLGGGVYASGMCAHPPFQIDGNLGFPAALAEMLVQSHDGMVRILPALPEDWREGSFHGLRARGGLSVDASWTDDAIEYTLRCTKPATITLIVDGADATQVRLSPDEPFKGLVRR

SEQ ID NO: 30 - protein, 389 aa, unidentified
MMNTNNTVCGANHDGAMSLAAPGDYGVACYRIPALAEAPNGWILAAFDARPHNCQDAPQANSIVQRISKDGGRSFEPQHVVAAGHDGVDKYGYSDPSYVVDRQTGEVFLFFVKSYDAGFGTSQAGVDPSARTVLQAAVTSSVDNGVTWSEPRIITADITNSESWISRFASSGAGIQLTYGEHAGRLIQQYTIKELDGRYRAVSVFSDDHGATWHAGTPVGDHMDENKVVELSDGRVMLNSRSSDGNGCRYVAISRDGGATYGPVIRETQLPDPENNAQIARAFPDAPEGSAQAKVLLYSSSSPSDRIDGLVRVSIDDGKTWSAGRRFTTGPMAYSVIAALSHKAGGGYGLLYEGDNNNIMYTRISLDWLNGQLNVDGIGGFPLSGEGGC

SEQ ID NO: 31 - protein, 545 aa, unidentified
MTFIKQMMPRYVASMTAGIVAAAMAATCAFAPVANADAVSPTQETIQSTGRHFMVYYRAWRDVTMKGVNTDLPDDNWISMYDIPYGVDVVNIFSYVPSGQEEQAQPFYDKLKSDYAPYLHSRGIKLVRGIDYTGVAVNGFRTFMKEQNKTESEATEADYDAYAKQVIDKYMISVGLDGLDIDMEAHPNDADVKISDNVIRALSKHIGPKSAKPDTTMFLYDTNGSYLNPFKNVAECFDYVAYQQYGSSSDRTARAAADYQPYIGNEFVPGLTFPEEGDMNNRWYDATEPYEESHFYQVASYVREHNLGGMFVYALDRDGRNYDEDLRRIVPSNLLWTKTAIAESEGMALDTAKTAANHYLDRMSLRQVIDDNAASADKARDMVGKAANLYETNKAVLGGDYGEGFSNTYDPTLEAGLLGIDISVLQQQIDKSSEIIGADTAESDAKTALRMARDAAIDGLTGKIYTADQVSAWSQALKAALDATVPVPTPDSTDQNGNRDKVTNHKVQGQPKQLSATGISTDIIVAVGVTLAIAGVALSLSRKLS

SEQ ID NO: 32 - protein, 301 aa, unidentified
VNNAVLTTNPLRRYCADPNLAIFDGRYFLYCTDDGVDNWDTTAFSVYVSDDLATWERYPALDLRDVPWWDGSDGAWAPSVVQRADGKYVFYFVAGSQIGAAVGESPCGPFVPEPEPIVREGTFDCHTIDPGVYVEDDGTRYFLWGNGIAWAAPLNEDCLSFDESRAFSWVPGDFREAIWIHKRNGIYYASWSENDAREPEYCVKYAMAESLHGPWSEPRVLIEQDPARKLYATGHHNIVNIPGTDEWIIAYHRFAYNPAGRWAGGDGCHREVVFAPLDYNPDGSLVPVRPQVGSYVRSLTF

SEQ ID NO: 33 - protein, 787 aa, unidentified
MTDNTTPAATAETLPYKNPDLPAGERIADLLSRMTLEEKVGQMMQLDARGGDLEDLIVNKHVGSILHTSPEDLSRAAKTVNEKTRLGIPLIIGDDCIHGYSFWPGATIFPSQLGMAVSWDAAKVEAAGRATAEEVSCTGVHWTFSPVLCIARDTRWGRVDETFGEDPMLIGELASAMVKGYQGGAKAGEALPKNAILACAKHFAGYSETQGGRDASEADLSHRKLESWFLPPFERVAKEGCGTFMLGYESIEGVPVTFNKWLLTDKLRGAWQYNGTLITDWDNVGRSVWEQHVKPDYVHAAADAVKAGNDLVMTTPQFYEGAIEAVKTGLLDESLIDDAVARILALKFRLGLFEDPRLPDAERIKAVIGSAEHQQINLELARESVALLRNDGALPFAANKAKRIAVVGPLADDAQNQLGDWTGNSGQVSWMPDGQPRGMITTVLDGLTQLTSDDCEVVYSRGANVIDLVPDPAGEFYPDGQPRPKIGVSAAVDRAMIDEAVANARQSDLIVAVVGDVVQLVGETCSTATLELLGGQNALLDALAAVSRETGKPMVTVLISSKPQVLPASIVGEYGVFAKRVSDPETGTGSILWAPNPGMRGGQAIAEIILGLTSPSGRLPITFPRHAGQLPVYYNQIRGQHGDRYADLTQDPAFAFGEGLSYTTFAYGEPTITGGASNADGTFAETDTVRAEITLTNTGERAGVEIVQAYIGDIVTSYSWTDRELKAFQRVALEPGETETVTFEIPVANCTIVDPDANRIVEPGEFELLIGPSSRREDLKRTTFTVA

SEQ ID NO: 34 - protein, 567 aa, unidentified
MSGTQAHHAGGTRARIGRAATGVVSSAAQFGSDRADIADSMGHTAAEMAGRAGMYGMSSTMHGVGWTAGKARRIMNRGKRALRSGKGMRKTAGKPKALSEAKPSEKIGKFAAKGKASKRIGKHIGAGLGNAGRSVKRMGSTGMGWMDEAGARLTVADDDFASKLGSATRDLTFKAARAGVKGVNSSAKFIWRHRRAPVKAARDVQATGQAAVRAARVAANFVRMAASRVIAGAASISLPILPVIAAMLAVLGVLLAVMGAFLGSNASESTVSGVPAEYEADVIRAGSICQVVTPSIIAAQIDQESNWNPKAGSSAGAQGIAQFMPSTWASAGKDGDGDGKADIWNPHDAIWSQGNYMCNLASQVETAKKSGKLTGDTLELTLAAYNAGLGSVLRYGMVPPFEETINYVRRIKELAATKYTATGTAEGGTVGSLEPKLTVSGGIVSTAGITPDTRYPWGQCTWWAATRRADIGKPIPGWGNAATWAGSAASAGYTVDGSPSAGSVIVFQPGVLGASADYGHVAMVEEVRGDGSILISESNALGLGVVSTREISASQLAAAGNGVRYIH

SEQ ID NO: 35 - protein, 693 aa, unidentified
IIMERKEFKWPQPLAGNKPRIWYGGDYNPDQWPEEVWDEDVALMQQAGVNLVSVAIFSWAKLEPEEGVYDFDWLDRVIDKLGKAGIAVDLASGTASPPMWMTQAHPEILWVDYRGDVCQPGARQHWRATSPVFLDYALSLCRKMAEHYKDNPYVVSWHVSNEYGCHNRFDYSEDAERAFQKWCEKKYGTIDAVNDAWGTAFWAQRMNNFSEIIPPRFIGDGNFMNPGKLLDWKRFSSDALLDFYKAERDALLEIAPKPQTTNFMVSAGGAGIDYDKWGYDVDFVSNDHYFTPGEAHFDELAYSASLCDGIARKNPWFLMEHSSSAVNWRPINYRVEPGELVRDSLAHLAMGSDAICYFQWRQSKAGAEKWHSSMVPHAGPDSQIFRDVCELGADLNKLADEGLLSTKLVKSKVAVVFDYESQWATEHTATPTQEVRHWTEPLAWFRALADNGLTADVVPVRGPWDEYEAVVLPSLTILSEETTRRVREYVANGGKLFVTYYTGLVDDKDHVWLGGYPGSIRDVVGVRVEEFAPMGNDFPGAMDHLDLDNGTVAHDFADVITSVADTAHVVASFKADKWTGFDGAPAITVNDFGDGKAAYVGARLGREGLAKSLPVLLEELGIETSAEDDRGEVLRVERADETGENHFVFLFNRTHDVAVVDVEGEPLVASLAQVNESEHTAAIQPNGVLVVKL

SEQ ID NO: 36 - protein, 786 aa, unidentified
MTDKLIATLDKAGVRAISTDLWGIFFEDISYSGDGGLNADLVQNGAFEYNRADSAEWSNYSFWRKIVPDGSFAAFCIHDIDPVAVENPHYATVEIEQAPAALDNTGWDGMVFHAGETYDFTAWMRVRSGSAMPVTVSLHGDDGSIISETTITVTASAWTTYQASLTVPESGEEDGKAGASVSTQGTLRLTFGGEGVIDLDFITLEPRTTYHGLKHFRPDLVKALADLKPRFMRFPGGCITHGLGMDNMYHWDRTIGPVEHRPHNFNLWGYHQSFRIGYYEYLCLCETIGAKPLPVLPAGVSCQNTSQGPVPVAQEDMPAYIDEVLHLIEFCNGGTDTEWGAKRAAMGHPEPFGLEYLGIGNEDLIDDVFKNRFQQIFDAVKAAYPDIVVVGTVGPSPSGKDYEEGWKHAREAGLPIVDEHSYQSSSWWFHNLDHYDHTDRKGPKVYLGEYGSWNTQLINGLSEAAFMGRMELNGDVVAMASYAPLFAKNSHHSWNPDLIYFDNERAYHPYSYWVQQMYATTTADTAWPVAVEGPSTLRRTLPDTVRLRIAGNAKADFNNLTVTTASGETVNLGDVSYDGRTIDTPLDLHTDAYSIDATVVYYEGKWGMDLICGDIDGKNHNIVSFGRGHSVRVVRDGTAYALAGTEVSMNEVLPGTTWQVHADVTGRGQAMKLYIDGTLIADGTEVKDEPRRTVTVSRNAKAGETYVRVVNAMDEPVDVDLSQILAGLDVSAQSAATATATILAGNDPYAGQVGEESPTKPVNTPLDLMDGTYAAPAWSFTVIALK

SEQ ID NO: 37 - protein, 1029 aa, unidentified
IRHSRISHYLYSDYILERKIMAKMTYDEKVTMRIERAISQHLTDAMYLHSVPVSITCWKVGGEPVPFAIARTAEYRNFNVGESWGAAWDTWWFHVTGNVPESWAGIEGTRPELVVDLGRIGLGPGFQAEALVRTSDGQVVKALEPYNAWVPLPEPGENFDFYIEAAANPQITPGFPYLPTDLGDEGVKDGPELYRMQRLDASLLDLGVWHLTQELRILSGLAKEVDPKRTRHADIMHALEQAADLIDPNDVHGSAPKVRHALVKVLSATAPEKGHTAFAVGHAHIDTAWLWPLRETKRKVARTFSNVVELSDEDKDLVFAASSAQQYKWLKENYPEVFVEVKRKVAEGKFIPVGGMWVECDANMPSGESLTRQFLQGVRFFEENLGGCSPVVWLPDSFGYSGAFPQIARLAGFKYFLTQKISWNDTNTFPHHTFLWQGIDGTEIFTHFPPSDTYNSNMSAADVAKTEIQYSEKGKGSSSLMLFGWGDGGGGPTREMLEAARLQKNLEGSPKVRIATPMEFFEQAERELRDPARWHGELYLELHRGTLTSQSHTKYGNRRCESLLREAELWATAATVLKRKAYPYEEISEIWQKVLLYQFHDILPGSAIAWVYSEVERNYRELEHRLESVISESIHAISGDGDKLMFANAAPLIQCGVPAGGIDVAVEPIASKVCMLDDGSIRMANERVSFVLDGKGHIISAVDLKTGRETIDPMQPGNELQLFVDSPAEWDAWDIEKNYERCLLDETVVSKVDLGADGTMVVNGVIGSSPYSQTIALDDDVDALTIRTHIEWKESDRLLKQAFPVKVYATEAKSEIQYGHITRPIHRNTTWDEARFETLAHRWVYVSEGGFGVGIANMTNYGNDIRQIIMSDGRPGTRIRLSLLRAPHFPAPRADVGSHDFAVSFTIGSSVDRAISQGYRLNVPARAVHGEHALAPFVSVEGDGVLVEAVKMAEDRSGDVIVRLYEAYGSQSRVVLHTGFGWKSVTETGLLERACDAVRPAVVDESRVQSNVELELHPFQIVTLRFALN

SEQ ID NO: 38 - protein, 706 aa, unidentified
MYPLGASYDGAGVNFALYSQVAQKVELCLFDEHDAETRIEMTEQNSYVWHNYIPGLQPGQRYGYRVYGPYDPMHGLRCNPNKLLLDPYAKAIEGNIDGDESLFSYWFKSPDDTSAMNDLDSAAHTMKSAVINPYFDWGNDQHPYISYHDSVIYEAHVRGMTNLNMDVPPDIRGTYAGLAYPSVIEYLKKLGVTAIELMPIHQFVNDSFLQEKGLSNYWGYNTIGFFAPHNAYSSSGERGEQVNEFKSMVKAYHHAGMEVILDVVYNHTAEGNHMGPTLSFKGIDNASYYRLVEGDQQHYFDTTGTGNSLLMRSPHALQLITDSLRYWVTEMHVDGFRFDLAATLARQFQEVDKLSAFFDIVEQDPIISRVKLIAEPWDLGSGGYQVGGFPSSWSEWNGRYRDTVRDFWRSQPSTLPEFASRLMGSSDLYQVNGRRPVASVNFITAHDGFTMNDLVSYNEKHNEANGEGNRDGESNNRSWNCGVEGPTNIPDVNDLRQRQMRNMFATLLFSQGIPMICGGDEVARTQQGNNNAYCQDNEISWTNWHLDKGRKELLAFVSKLIHLRLDHPVLHRRRFFTGREPGDDSNMIPQVEWFDHTGSIMDMDDWQNTHAFSMMIYLNGSDIPEVDWYGNRMVDNDFILIFNAHYEPIMFTLPDERYGRKWQLVVDTHNPDGPELSYEAGFMITAQSRSFLMLMSDKKPKKPMGL

SEQ ID NO: 39 - protein, 746 aa, unidentified
IMTDAYSNDAATSTGTIADTTNDAASGAPQRAESTERLARPLVKLAKACGLSTSFIDQLGTYTEISDEALVAVLKALDVDASSDEAIKRSMTELEAENSKRLLPPTIVATTGKPTSLTLNCPSDADITASIELEDGTAFGHFALLPNLNSGKPDLTIAPDLPMGYHTLTVTVDGREGKAAIIAAPARIPVPEAVAEHQRWGWMTQMYSVRSRESWGIGDYGDLKLLLADAADKSKADFMLINPIHAGAPIPPLEPSPYLPESRRFLNVTYIRPQDIPEYGTLPADIRAQVDALHDSVAARNDESNPMDINAAWAAKRPALRLIFDAGRNNKRELEFEHFKTTAGPDLDSFATWCLCFEVWGAPWGENRWFFEKTIDDPAVRQLVEEHHDLFEFNRWLQWIAAEQVNAAQQEALDHGMTLGLMQDMAVGVHGLGADAWANPERFASGGVTVGCPPDFYNQQGQDWGQPPFSPRYLEATGYQVYREMVHSMYEHAGAVRIDHVLGLFRLWWIPQGLGALNGAYVTYNHEAMLGVLAIEATRAGGMVVGEDLGTVPDYVRRILADHGVLGTDVEWFNRVDDSPNAGDPYRAPEDYRKQALASVTTHDLPPTAGYLNFEHVKLREELHLLSEPVEAFAASAMAERTAMMNRLVENGYISQTVADDVEGHVQEIVEAMHAMLTDTPSLLLQAALVDGVGECRSQNQPGTSREYSNWRVPLADSSGHVVHTDEVFDLPRVQSLAAVMRREKR

SEQ ID NO: 40 - protein, 1023 aa, unidentified
MTDVTHVDRASQAWLTDPTVFEVNRTPAHSSHKWYARDPQSGQWSDLKQSLDGEWRVEVVQAADINLEEEPATAESFDDSSFERIQVPGHLQTAGLMNHKYVNVQYPWDGHENPLEPNIPENNHVALYRRKFTVSAPVANAKQAGGSVSIVFHGMATAIYVWVNGAFVGYGEDGFTPNEFDITGLLHDGENVVAVACYEYSSASWLEDQDFWRLHGLFRSVELAARPHVHIENTQIEADWDPEAGTASLDAALTVLNAADAATVRATLKDADGNTVWQTTGDAKAQTALSSGPLQGIAPWSAESPTLYELDVDVIDQSGDVIECTSQKVGFRRFRIEDGILTINGKRIVFKGADRHEFDAERGRAITEQDMIDDVVFCKRHNINSIRTSHYPNQERWYELCDEYGIYLIDETNLEAHGSWSLPGDVLTEDTIVPGSKREWEGACVDRVNSMMRRDYNHPSVLIWSLGNESYVGDVFRAMYKHVHDIDPNRPVHYEGVTHNRDYDDVTDIETRMYSHADEIEKYLKDDPKKPYLSCEYMHAMGNSVGNMDEYTALERYPKYQGGFIWDFIDQAIYATQPDGTRSLRYGGDFGDRPSDYEFSGDGLLFADRKPSPKAQEVKQLYSNVHIDVTKDSVSVKNDNLFTATGDYVFVLSVLADGKPVWQSTRRFDVPAGETRTFDVAWPVAAYRADARELVLQVSQRLAKATDWAESGYELAFGQTVVPADATATPDTKPADGTITVGRWNAGVRGAGREVLLSRTQGGMVSYTFAGNEFVLRRPAITTFRPLTDNDRGAGHGFERVQWLGAGRYARCVDNVLEQIDDSTLKGTYTYELATAQRTKVTVSYTAHTDGRVNLHVEYPGEQGDLPTIPAFGIEWTLPVQYTNLRFFGTGPEETYLDRKHAKLGVWSTNAFADHAPYLMPQETGNHEDVRWAEITDDHGHGMRVSRADGAAPFAVSLLPYSSFMLEEAQHQDELPKPKHMFLRVLAAQMGVGGDDSWMSPVHSQYHIPADKPISLDVDLELI

SEQ ID NO: 41 - protein, 705 aa, unidentified
MPTFEYTVDAATLCLTLIPAPMTLEYTHGTAMIRSLVTIEKRIPEYAATEGADETWETLPIEQLSSELERYCGVTVRTRRVRAATGEDDASANAAEDARDARAGAPAAMNGTVISLGLDSRLAHDEYTLDVFASDTIAVRGGGESGLRYGLQTLRQIIGQTSRAIPCLHIQDKPAFAVRAYSLDVTRGRVPTMEFLTWFVDQLALYKYNQFQLYVEHAFAFVELSEAWRGTDPLTAADITYLDEYCARRGIELVPSLATFGHMYMNLRTREHRGLGEFPEDADRPFSFIERMEHHTLNAADPKAHDFASRLIEEYAPLFRSKSFNIGGDETFDLGRGRSAQDAPEAGRDELYAGFVRDLCETLARHGRQPMLWADIALESPRTMDLLPGDITMLNWMYEPQIDESNIQTIAAQGRRQFVCPAVRAWSRFFPDYAGAWLNTYHMALAGVKYGAEGMVVTDWGDYGHVNDPRLSVPGLCYGAQNAWNPIEIDAHEMNRRISVLVYGDESGRIMDGLVRIDSDGVSFPWDLAVQVLELEYGSGTGMLNTDVASYVERSCGGELVFDRTLGCADARRRLLLRNHARLERRRDCDRALIDCGSAVVAVLDGSAREGLNPELLWVMLDGQRLFNRLGEELLVLAGGEDACDTKDVTGRALDASRRARLAADLELWFERYRVQWLSIGRYAELARIAHVVWSFADILRRGAL

SEQ ID NO: 42 - protein, 467 aa, unidentified
IIMPKLYYQFPGTWFGDCMPFGKGNEFFLYHQRDNRNPEPFGEPFGWDLATTSDFVHYKDCGVAVPRGRDDEQDQFIFAGSVFEGEGQYHIFYTGYNRDYPQRDKPAQVLMHAVSDDLYHWTKTQNAITFTPQEGYDPDDWRDPWVIRDDEHDQYLLILGARKIGPKTQQTGRTVKFTSKDLKNWTFEGDFWAPNLYTMHEMPDLFKMGEWWYHIVTEYSDRHKMVYRMAKSLEGPWIAPDDDAFDGSAYYAGRTFELGGRRILFGWVATKEDCDDVKNFEWAGTFVPHEVYQRTDGTLGVRIPDTVWDAFNEREQIVTNAVIDGTDARRNAVLAEGCGDLYSFEADVMFSEGTRSFGLRVYRDEASERGYQFIFPIGEHRYVFEGSPNFPWFNCMNIGLERPIELVAGREYHIQLIVDDTIATLYVNGVALNARMYQHPGDALSMFVTDGTLTVTNATISRGLKEV

SEQ ID NO: 43 - protein, 605 aa, unidentified
MTANNLNDDWWKQAVVYQIYPRSFKDVTGDGLGDIAGVTEKMDYLKSLGVDAIWLSPFYPSDLADGGYDVIDYRNVDPRLGTMDDFDAMAKAAHEADIKVIVDIVPNHTADKHVFFQEALAAEPGSPARDRYIFRDGRGEHGELPPNDWQSFFGGPAWARVADGQWYLHLFDKAQPDVNWKNPDIHEEFKKTLRFWSDHGTDGFRIDVAHGLAKDLESKPLEELGREYSVVGVLNHDFSHPLFDRREVHDIYREWRKVFNEYNPPRFAVAEAWVVPEHQHLYASMDELGQSFNFDFAQANWYADEFREAIAAGLKAAAETGGSTTTWVMNNHDVPRSPSRYGLPQIKGAPYHQLPHDWLLRNGTTYPEDRELGTRRARAAALMELGLPGAAYIYQGEELGLFEVADIPWDHLEDPTAFHTAQATMDKGRDGCRVPLPWTAADEPALADFSRPTPADDGTGENHVPLCAAGQFGTGASFGFSPATRAEGVTPAADPHLPQPLWFKDYAVDVEQADPDSMLALYRAALAIRQESLTATRDTTAEQVDMGDDVVAYTRAAVGGRTFTSITNFGDDPVVLPEGDVVLASGPLTPDGQLPTDTSAWIITQ

SEQ ID NO: 44 - protein, 606 aa, unidentified
MTTSQRTVIPDSIRTNGATPNPWWSNAVVYQIYPRSFQDTNGDGLGDLKGITSRLDYLADLGVDVLWLSPVYKSPQDDNGYDISDYQDIDPLFGTLDDMDELLAEAHKRGLKIVMDLVVNHTSDEHAWFEASKNKDDEHADWYWWRPARPGTTAGEPGAEPNQWGSYFGGSAWEYCPERGEYYLHQFSKKQPDLNWENPAVRRAVYDMMNWWLDRGVDGFRMDVITLISKRTDLSGKLPGEYGSELDDLPVGEEGYSNPNPFCADGPRQDEFLAEMRREVFEGREGFLTVGEAPGVTAQRNEYITDPANGELDMLFLFEHVDFDCEGVKWKPLPLDLPKLKSIMAGYQAAVKNAGWASLFTGNHDQPRVVSRWGDDSSEESRVRSAKALGLMLHMHRGTPYIYQGEELGMTDAHFTRLDQYRDLESLNAYRQRVEEAKVQSPESMLAGIAARGRDNSRTPMQWDGSVYAGFTAPDAAKEPWISVNPNHAAINAVGEFDDPDSVYAFYKQLIALRHDMPVVAAGDWHLLDADDAHVYAFTRTLGDEKLLVVVNMSGRTVDLPRETAELAAAGITESNVVISTYDAPHAVASLANRELDPWEAAVIQL

SEQ ID NO: 45 - protein, 798 aa, unidentified
MVEKKRFDGADAKGATGMLRALRARATASGAVRGVTAAMASVAAMAMLGAVCVTPASALADVNADADASGVRSAASVTASAAASDGTSSDARSQARSATSATRPVANGVMVADETAADAMPDNPDAELPDKVSAEISDDATVVSEQYAATPEGELKDIKTGETVTDPKIVGTESKQPDPLAKTDGESFIPVSAADVKEKVAANGGDVNAVSSKTRATNASVKLAALQNNEYGAHWGTYNGTQAFFDARNNLFAQQAKGVIDVSVWQNTIDWQAVKNAGVEGAIIRLSYGWGNGFDAQAKRNISECKRLGIPFGVYVFSYAESAADGASEGADVVNLLRQAGVNPGDLSYPVFYDLENWTYTGHRSPTSPSVYDGIVNSWYGKLQAAGYNNLSVYSYTSYLNSALNSGNIHGKTRWVAQYGSTMQYTAFPTNDRGWQYTSGGSISGISGRVDMNAFGNKTPVTQYVEVEKDGKWYYVDTATGKKATGWQRLSSSGGKWVYYDLSDGHMLYGEQQIGGKWYYLNPGTGARATGWVYLKVGANKWVYYGSDGAMLYGEQNIAGKWYYLNPDSGARMTGWVVLPDDRNVYYAPYMLYGEQKINGAWYYLNPVTGARKQGWQRLNSNGGKTVYYGADGKMQYGEKTIDGKQYFLDSMTGARLSGWRRTKITNEPSGGKWVYYGANGAMVHGESQVRGHWYFFDDDSSSSNGQVVYGWKYLSSSGGKWVYYQQGLGVMLYGEQHIDGAWYYLDPVTGARKSGWQRLNSNGGKTVYYGADGKMLYGTQYINGRLYYFDSVTGAKR

SEQ ID NO: 46 - protein, 780 aa, unidentified
VQCKSTHVEGDNMAENTSTFTGAAADGTALTAVYLTQPAANVAIGLVFAGSDLPHIVHWGRPLAKPDTLLAAYDALKPQRVSGALDDTAWPSILPTQAESWIGEQRVVLRRAGVELFPKFTVTNIEAGGVLEATLDAVSGESYTDVAGHARATGPVRVPGVIVTARDEEQGVEVEWHLELLPGGLVRQKATVTNLFGADAGAPLEIGKIELGFPLPESAGEILTTTGHHLRERSPQRQPLTVGRFEKPQLAGRPDFDASLLLTAGVPGFGFEHGDAYSVHVGWSGNSVLSAERLPYTTGVIGGGELLFGGEVTLAGPGEGQNSYDTPWLFGSYGDGLNEIAARFHSYVRSLHPRLFSHGRPVILNTWEAVYFDHNFDTLKALADKAADSGVERFVVDDGWFGSRRDDTSGLGDWQIAQDVWPDGPKSLKALADYVHAKGMEFGLWFEPEMVNPDSDVARNHPDWILSPTTGRLPLQGRTQQVLDLTNPDAFDYIYGCMDQLVGELGIDYIKWDHNKLVTEPGSRRSGRPAVHAQTLAVYNIFKGLKTAHPGLEIESCSSGGGRVDLGILEHADRIWVSDCVDPVERADIQRYTSLLVPPAMMGEHVGASPAHSTQRATSQELRMAMAFFGHMGIEWNLLKEPDEALAKLAVWVAEFKKHRDWFAIDTCVHADSNDPAVRLDGMVMPNRDAAIYRFTQLTTSQTYPAAPVHLPGLDPERTYRVSPLDPSLDLTGLANGQSTLGWWNEEGVVLTGEALQRYGIRPPSLHPQQAVLLKAVALS

SEQ ID NO: 47 - protein, 751 aa, unidentified
MTSTGRFTLPSEENFAEKTKELAELWGADAIRNSDGTHLDEAVLALGKKIYSAYFPTRAHNEWITLHMDETPQVYLLTARILAESNAVDVPLMDGFFEEQLKPNRDADPHKYWEVVDRTTGEVVDPSGWTLDPGEDTVHVTAAVPMHEYTVSFLAYIIWDPVEMYNHLTNDWGDKEHEIPFDIYHPATRKFVFDTFEQWLKDSPQVDVVRFTTFFYQFTLLFDAKRREKVVDWFGCACTVSPRALDDFEKEYGYRLRPEDFVDGGAYNSAWRVPRKAQRDWIDFLSGFVRENVKRLADMSHAAGKEAMMFLGDQWIGTEPYKDGFEKLGLDAVVGSIGDGTTTRMIADIPGVKYTEGRFLPYFFPDTFYEGNDPSIEGLDNWRKARRAILRSPIGRMGYGGYLSLAAKFPKFVDTVTHIADEFRDIHDRTGGVAAEGELNVAILNSWGKMRSWMAFTVAHALPNKQTYSYYGILESLSGMRVNVRFISFDDVLAHGIDSDIDVIINGGPVDTAFTGGDVWTNPKLVETVRAWVRGGGAFVGVGEPSSAPRFQAGRFFQLADVIGVDEERYQTLSVDKYFPPVVPDHFITADVPVDPAAREAWEQAGYRIPLSGCGGGQSIKPLGGIDFGEPVLNTYPVNENVTLLRADGGQVQLATNDYGEGRGVYISGLPYSAANARLLERVLFYASHNEDKYAAWSSSNPECEVAHFPEQGLYCVINNTDQPQRTTVTLADGTTEDFDLPDSGIAWREA

SEQ ID NO: 48 - protein, 432 aa, unidentified
MTTTAIPKSVSDFMQHITDLCGTEHAAWAENFNACFANTLTTTVKRHDDGTTFLLTGDIPAMWLRDSTAQLRPYLALAAEDGDIASLIAGLVRQQFRYITIDPYANAFNEEPNGASWDKDDQSDFSSPWLWERKYEVDSLCYPIQLAWMLYANTGNTDHFNDEFIAGVKKILDVFETEQHHENSPYFFIRDTDIPTESLSNDGKGLPVAYTGMTWSGFRPSDDACTYHYLVPSNMFAAVVMGYLERIFGGEILDDADIAARAGELRRTITEGIENHAKTTNRNGETIYAFETDGLGHVNVMDDSNVPSLMAAPYLGYCAPDDPTYLATRRTLLSDENPYYYEGEYLKGIGSPHTPPHYVWPIALSIEAMTSDNKDFKAHILDRLVTTDAGTHLMHEGIDVDDPTKYTREWFSWSNMMFCELVMDYFDIHVKH

SEQ ID NO: 49 - protein, 631 aa, unidentified
MEFTVSGTTVRFDERTMQFAFTRDGAEWNTCADFKPTLQCAQGTFAFADATSITHEQRETGTGTGIRSIFTGFGHSAYSFETYVWVERASGDVLFEWIPLNEQGLNITNVTWPAAMDFDCADDHDTTLITHEQGVMIPNTWPTAVSTKDIAFDGRFETAGGYMPWFAQLRADGHGYIAICETPWNAGYGIDHPSDGPYTHINTWFEPSLGTMNYRRVVRYQFLDHADHTAVCKAYRSYVNERGRLRTLAEKAARNPSVRDLIGRSWVHIGIKTKVQPDSYYYDKDHPEKNDSLVTFAQREKQMRTLHSMGAGRLYMHLDGWAQPGYDNAHPDYLPACQEAGGWEGMKSLVDACHEQGDIFGTHDQYRDYYFTAQTFDANNAIRLADGTMPEHARWAGGHQTYLCAELAPDYVRRNFAEIAAHGIKLDCAYLDVFTCNEGDECSNPEHRMTRRECFDRRAECFEYLLSHGILSSSEEVSDWAVPSLIFCHYAPYDFQMRSPNEPRQGVPVPLYNLVYHDCIIEPWMMERVANGDDYMLYALLNGGAPYLIRDAAYIGVDGDMDDEQRARTENDIERCHTVAAFHERVGMQELVRHEFVDDDPLVQRSVFADGTAVTCDFHTQTYRITDCPHH

SEQ ID NO: 50 - protein, 508 aa, unidentified
MKNKVQLITYADRLGDGTLSSMTDILRTRFDGVYDGVHILPFFTPFDGADAGFDPIDHTKVDERLGSWDDVAELSKTHNIMVDAIVNHMSWESKQFQDVLEKGEESEYYPMFLTMSSVFPNGATEEDLAGIYRPRPGLPFTHYKFAGKTRLVWVSFTPQQVDIDTDSDEGWEYLMSIFDQMAASHVSYIRLDAVGYGAKEASTSCFMTPKTFKLISRLREEGVKRGLEILIEVHSYYKKQVEIASKVDRVYDFALPPLLLHSLFTGHVEPVAHWTEIRPNNAVTVLDTHDGIGVIDIGSDQLDRSLKGLVPDEDVDNLVNTIHANTHGESQAATGAAASNLDLYQVNSTYYSALGCNDQHYLAARAVQFFLPGVPQVYYVGALAGRNDMELLRRTNNGRDINRHYYSTAEIDENLERPVVKALNALAKFRNELSAFDGEFSYEVDGDTSITFRWTAADGASTAALTFEPGRGLGTDNATPVASLAWSDAAGDHETHDLLANPPIADID

SEQ ID NO: 51 - protein, 427 aa, unidentified
MDSSRTMPDWVKYGVFWHVYPLGFCGADIRPAGPRQFHGRGLDAIIPWLEYVRDLGASGLLLGPVFESATHGYDTLDHMHIDVRLGGDAAFDQLVAACRDMGLQVMLDGVFNHVSRNHPAVQTALDETAGRLNPADNPWHGLVRAHVGDNGEPALDVFEGHGDLVALDHSADETVDYVAHVMSHWLDRGAAGWRLDAAYAVPSPFWARVLPQVKAAHPYSWIFGEVIHGDYPRIIEESTMDSITQYELWKSIQHALETENFFELDWNLKRHNAFLDSFVPQTFIGNHDVTRIASQIGPAKAALALAVLMTVGGVPSIYYGDEQGYVGVKQERFGGDDDVRPKFPDSPAELSTLGEPTYRLHQALIALRRRNPWLLDARTEAVKLENKHFVYRSTSADAQHSLTVDLNIEQSPTFTIRNADGSTAYQY

SEQ ID NO: 52 - protein, 746 aa, unidentified
MAAVQHRATTRTSNTDNSTTKTKSKATSARKSPATKRKRVSAETARAAAALKGLAVEAPAPSIEANEPGQFGRINVMDITPAEERGIFPARVELGEPFEMTAQVFIEGRTKVGATAIVRNPRGKETMRRAMTCVNPGLDRWTVMVKCGEHSDLKPWEDGYAAVKRQLGEWTVTIEGWEDAYVSWLHDARIKVRVMDDVDNALNSGAELLARWAETPDTGLTARDRKTLEKAAETMADQTLSAEDRLAAGDNPTIAALHETHPLRDGISPSQPQRFKVERPKSSFAAWYQFFPRSEGATIDPNTGKIIQGTLKTSMAGLERAAAEGFDIVYLPPVFPIGVTNRKGRNNTLVAGPDDPGSPFGIGSELGGHDTVDPLLGTMDDFKALCQRAHELGLEIALDFALQCSPDHPWVKAHPNWFRHKPDGSIAFAENPPKKYQDIYPIDFNADMPGIEKEVERIMNLWIEAGVTIFRIDNPHTKPVRFWQDVIAAVTKKHPEILFLAEAFTRPGMMRALSYVGFTQSHCYFPWRNTKDELEEYLPVTNGDDGYYQHNTFWPTTPDILTAYVRDNGVAGHCVRAVLAAMGSPSWGIYNGYELIENKQRPGFEEQIDNEKYEVKVRDWSKAKQYGVAEMLTALNKIRRAHPAALSYHNLTVLPTSDPNILAFARHTPAELTGTGQADTLIVVVNLDGHNAHQSMIHLELSELGLPTDRPLNVRDELTGREFQWGWDNYVSLAPWADVAHILSVQ

SEQ ID NO: 53 - protein, 552 aa, unidentified
MAKETKIKEDTVTVPVAQGDLDAVSNAEFYNPHGVLGGHLGIGKHADTATIRVLRPLAKSVTILTQDGEYPMTHEYNGVFVTTVPASGTKKQPTIPDYRVRTEWESGAVLIEDDPYRYMPTVGDMDTYLFGEGRHEKLWEALGAHVLRYDDPMGGVDGTPGEQVVGTAFSVWAPNAHAVRVVGNFNAWDGRRHAMRELGSSGVWEIFIPGIGAGETYKFQILNANYTWEMKADPMERQHEVPPNTASIVTESTYEWNDDAWMQHRRTTNPHDGPVSIYEVHAGSWKQGLTYRDLAKQLVDYVRQEGFTHVEFMPLAQHPFSGSWGYQVTGYYAVDSRLGSPDDFRYLVDQFHQAGIGVIMDWVPAHFPKDAFALGRFDGTPLYEDPDPLRGEHPEWGTYVFNFGRREVRNFLVANALFWLEDLHVDALRVDAVSSMLYLDYSREPGQWRPNIYGGRENLEAIDFLKEATATAYKNNPGVMMIAEESTAWPGITAPHLRRRHRLRHEVEHGLDARHPRVSARGAHQPQVAPQRNHLLHGVRLLRTLRIAHQPR

SEQ ID NO: 54 - protein, 426 aa, unidentified
MKFGVNYTPSHGWFHAWLDPDWDGIDNDLKQISELGMDHVRIFPIWPYLQPNRTWINKKGVADVRRMVHIAGEHGLDAYVDVFQGHLSSFDFLPSWLVTWHAGNMFTDADAVAAERELVKTMTDELSKEPAFKGLTLGNEVNQLSDRPHPTKMSATGRQIDAWLDALLPTAAGEGHNALYSVNDGTWFIDGHPFTPVQSATKGDMTVIHSWVFNGIAQGYGATSEECSSYALYLAELAKAFGKDSERPVWLQEVGAPENVLETDYTPEFCRKTVERAMDCRNLWGVTWWCSHDVPASMEDFPFFEHSLGLFDEQGQLKPIGRTFGELAAQYRSALPAQPKTVAVVIDVDEAGNPVNRSALGPGGSVCDLWMKLQVAGQRPTIITSQVAANQEALAQRGILELHADEHPYAARYYTAVSDPSFETAD

SEQ ID NO: 55 - protein, 275 aa, unidentified
DAGLYWVGSGVDNQALFDFDGKALASLPTFKYIHTGAVTDHVFTKIDPVEIIATDSDSIDVIKAQLPSEAAAHYQDGVDETETVTWQSAALDWIRGAGTYTITGNLPADAWGNVDKVSLVKKTETPVKPSTENLDKAVAESGEINRDEYTNESLAKLDQALAADQFMLDYDFSAPADAAKVTVTLKSNPNVTETYTVAVASRAEGGSGSGSDGAGNGGATINPDTGEGDKTNGANGDEITGVLSNTGSAVTAVGLAVVVLGVAGGVSLALRRKCS

SEQ ID NO: 56 - protein, 401 aa, unidentified
MASGALGVGERINGVNLGNWLVLERWMKPGIFAASGEADEIWLHRATKSAELEALLTRHRDTYITEADFRNIAAHGCNLVRIPVPYFVFGDVPGHPGCTEYLDRAFDWAERAGLKILIDLHTVPGSQNGFDNGGLTGVVRWHRSPRAVAYALDVLVRLARRYRDRAALFGIEVLNEPIDWLTYAMSPSSRQAKDRSEARGSGPIPMAFLKRFYRETYHRLRPILAGNQVIVFHDGFRLGRWRDWFVREGMRGVMLDTHVYLVMAEQFPLFRLIPDRWLMGWYRLFVRWNERRIRRAARYTPVIVGEWCVANGLVNRVVDKRTGDGGRSKENAMHSASIRGSIYREVAAMQRKAWNVSAGQIYWSYQLRGNRDFLPTIDPQSDTSRLDPWDLTHVWHAGWMA

SEQ ID NO: 57 - protein, 581 aa, unidentified
MTATTSTTEPTTESPADERFVEHPLAPGRIAIHDPAIVEDNGTYYVFGTHRRCARSTDLVHWERFENNLTRDPASLLGGIWEAWPKQPENPALEGNTWAPDVIWNDVMRKWCMYLSVNGHEFRSVIVLLTADRLDGDWTYVGPVVYSGFNVDNVGRTDVPRVLGDEAAHGDLSRYASLKDTRINAIDAAPIRCDHGELWMSFGSWFGGIWMFKLDPKTGLRDYSVRYPLVHDSADPYYGVKVAGGYWNSGEGSYFVHRNGWWYLFMAYGWLGLTGGYQIRLFRSRNLVGPYVDQNGNPAISNGEIPDNQTKDTGIRLTSSVKWSGGPADDDTVEVSQGHNSVLTRGSDGRLFLTYHTRFIDRGDEDYETHVRELLPTADGWLTAAPYEYRGSAAVPPTAANTSDPTVGDDDDGFGDYIVAPSSAPNLAARLCHCVRPVHESDLAHPSLVSGDYEFVRHDPHAYFNGTRNADGTWHGVNLPETITLQPDGRVTSGGVVDFFDTPDANLPRSTDGPVSRGSWRMLGATPAGVHCCDSDMAITIDGMTYTGVFAVLPRETDGRATLTFSAIGGNQAIWGARIEG

SEQ ID NO: 58 - protein, 344 aa, unidentified
MTVYNNPIVLQRADPWVLKVDGEYWFTASDPEYDKIAIRHASTINDLQKAPETVVWRKHESGVCSKYIWAPELHRVNGAWYIYFAGAEHEFEPNGMPTHRMFVLENTDADPTTDNWVEKGQIVTPMDSFSLDATTEVIDGVQYLVWAQKDYDIPGNSNLYIAPMANPWTLAADPVMLTKPEYDWECIDFLVNEGPAFLFHDDKIFITYSASGTGVPYAVGLLTADRGSDLMDPKSWTKSPVPVFKTCSENGQYGPGHNSFTKSEDDSEDLMIYHARNYTEIKGDPLFDPNRHARVGVVRWNEDGTPGFGVPEPDNLWTPVTTDVLPPDGGPKAGTPAVGLAAEA

SEQ ID NO: 59 - protein, 886 aa, unidentified
LGKQPTTNSTNLIDTGMSTVPQVTILNPAAATDGTYADLGDNASTRVNAGAETGGEDNGYAKWGDVYLQYDLGENRSITAVNLYHNGYENAVSIFKQVKVEVSNTADFSQSTVISGPADYEETTATHLAAQKIVPTGGQIDARYVRIWQRGHYIRNTNSSWTGYSNGVQFREIEVLAKLKDSETPPAQEELRNIALGKTPYVYGLAPTNIAAINDGKMDSNYAVHNSLGLHWLQFEYKNTYRIRKIVVKLEPGTYDSIRVSIASTPSSRGTAVYEHSNVTVGDKPIVVETDMQGSDVRFTVNRNASSPTKYSEVQIWATGNSYDESRTQYVSPESKYDTLAWSDEFNGDSIDESKWNIIDGMANHGAIYNRDAVGITKDGDNSYLAINTKNYGSTQALKQAVGYDDYGTQLNEKKVTWSSGRLESKNKYSFQNGRMAVRAKVNDSQGIWPAIWMLSQDETGHDEIDVLEYLGQDPWTAWTTNHFGILDKNKQSDGKANTNWEAWSQAFHVYEVEWSPERINWYIDGRSVFTSTRGRDDGRDGMHTRPMFPILETQVGDGWVGDVDYSRQNTKQNSDFLVDWVRVYQRADQDQVRFDDLEGLSDAKQFPAGAYRIGSSAATDGLETVSNGSKQWQNKNNFFYEGQPRYETNRVMTKEDADADQSITWFVPGVKDAHLTAYYQTIYDRHVWSNAANGWHGYSIRSGLNDGANIDFRVETSKDGATWEPAKVSVVDNYIADHPAYARTTFDAYSLPEGTKYVRAVFPNLNGVTYSTAAGRRLPVLHTDVQLAKATFLQSKEHVNDVEQTDTPSGNTGGNAGTSGSQPGVPSGKPSLPSSAVGEQHHAVGLTQTGAPVIWPVALSALLVTIALVIVLCVHSRREAEPHDE

SEQ ID NO: 60 - protein, 1606 aa, unidentified
IERKESGMTSRQGRQAIAATAAMGVAVALALPTAAFAQSATQGKETATTTSSGTTYYVSSAHGDDANAGTSENAPWKSLTKVNDIASDLGPGDSVLLEYGSEFNDQYLHIKDTAGNADAPITISAYGDADEGKPVIASNGVKGSQWEQDYRANVGNHKNKGTVSTTLLLKDVSYITVSNLEITNDDADVYDPIDTWKWTDTPDSDGTKLDRSASRMDRTGVAGIAENGATMSNVTLDNLYIHDVDGNIYNKHMANGGIYFMAHYPMENTSAETDVWLREHVSRFDHVTIRNSTVKDVDRWGIAVGYTAYLNYIDANYGDGSIDDALIAKYGSTNVRIENNYVKGAGGDAITLMYCDRPVIEHNVGDSVSKHINTQDYTQPGSYGGRVAAGIWPWRCKDPVFQYNEMYNNLNAEHGNGDGQAWDADYGDGTLYQYNYSYGNSFASLMICNWYAVNTTFRYNISQNDRQGVFDLPSNGPGNHIYNNTVYVDADSQVLTKRSNSQSLFENNIFINATNTKKTETWNRGSQNGGQTYDNNMYVNYANKPTSDANAIEADDVSAVLAGAGSAPTSALKSGAEHARTGEKAAFDGYRPVAGSKAINAGKVVSDLNDYAVENDFLGNAVKGRPDLGAVESDVVSVTMASSKYETGTETDSGTGDKTKVIHVTFTDKNPVTVKELLSNVSADKGVDKAVYRVADAKSGKSADARSAESEPNMLDRLLSLLPGSDRNAKDDETKLADSEPVRDGDILRFSAEGTDETDEYTIRQRITWDWVADYEQGVADFDWKAQRRTSAGGEWTTISAYDGSWPNTVYDQYYGVGVNGTLAELSGDRKQTHGLLIDKPGDGLPTAMAWKAPESGTVMLSLKTFADKIAEPYLRQNADNAGKKVTLSLMRNDETLCSADDLSVYQKSSEQFAQCLAEHGSIDVQEGDWIRIVADAETGVKAPSLHISPVITYEDKAPAAPKQNVRYDVSYAATDAVVGTQSAVAAAFTADGGEADAPDGVAFAFKDGGDEGEASPVIDASTGAVTFTPAAGQYGATVTRTVVVTYADGSSDETTVTFRVAQSHAQRLNVLYPTVRGDAGTDLKRTPKFTLKADGAAASVPEGTTFALGANAPAGASVDMANGTVTLNSGVGGTVTVPVTVTFADDGASVSSTARFEVTAPAALGSSELETATVDGVNVVYAPFSADSPMTVAQLLAKVTAEPSGADKGVYRDGVRLEAGAELAENDVLRFSAKGSTVSDDYVVKSKTTWDWVNDFQVRVQGPIWYGQRQTEADGVWSDIADFDATYPNWMYETYYGPGVDYANHSLPTDRSAIHGLISDSPASAGGSAMAWKAPKAGTVKVSIREDEPYLRQDGSNGKALTLRLMHDDKVVCFADLTVSKQRSEEFANCVADKGEIAVEAGDWIRVTATSASGMNKPSAHISPVIAYMAASTPGPEPVPVDKSTLKATVEEALGLAESDYTDESWAALVAARDAAQTVLDDDAATAEQVETAQNALRDAIDGLEKKPVDPDPNPKPDPNPDPDPTPDPDPDPGPDTKPGDGSGNGSGTGNGSGSGNGSTGSGSDGATTGGKLTATGADVAGAAAMVALTAAAGIGLAAAARRRR

SEQ ID NO: 61 - protein, 423 aa, unidentified
MTMTFPEGFQFGTATASYQIEGGATEGGRAPSIWDTFSHTPGKTVNGDTGDVACDSYHLWQKDIDLLADLGVDSYRLSVAMPRVMPTEDGPVNEEGLDYYERVVDALLDKGIKPTVTLYHWDLPQYLGDGNGWLNRDTAYKLADYARIVAHRLGDRVETYTTLNEPWCSSYLSYGATEHAPGLGLGPGAFPAVHHLNLAHGLMAQAVRSEVGDRSQLSVTLNLQFNRGDADAVHRLDLIGNRVWLDPMLRGYYPNELFAITKGICDWEFVKDGDLEQIHQPLDVLGINYYSSGLVTMSGRPQFPQSTGPSTAPGASDVDWLPTPGEHTDMGWNIDPKGLYDLLMRVHNDYPEIPLMVTENGIAVEGGDRVVTEADGTKAVHDPKRIDYLKRHFEAALKAIEDGVDLRGYFVWSIAPAAKQSGY

SEQ ID NO: 62 - protein, 344 aa, unidentified
IMANTIKVGIIGIGAIGSDHLNRLANTIPNVEVVAVNDVVPGRAQAALDKFGIEAKDYADYHEVINDPEVEAVVITANNEAHFEIVMASLEAEKYTFCEKPLALAADECLKIMELEQSKGRRLLQVGFMRHYAPEYQQLKKIIDSGEIGLPLMADCHHFNQMQPANYDSSRNIVETAIHEMDIMHWLLNDTYKNVKVYLPKATSAVQDPSIHDPQVIVIETTTGINIVLRAFVRCQYGYDIKCDIIGEKGVAELPTVAQVAIRKDARYSTAILTDWKERFITAYDYEFEDFMKRVAAGRPPVGPNAWDGYQAAVTADAALRSIANGGTTEAIELIDAPAFYTAE

SEQ ID NO: 63 - protein, 674 aa, unidentified
MMSTATGFPNRQINLDFHTSPYVEHVADDFDAEEFAERLHRSHVNSITCFARDHHGYLFYPSKRHPELVHPHLSDRNLLLEQIDACHKRGIRVPVYTSVGWDGYSALNHPEWLARDPQDNVINGCPSVPKPNFYDSLCLNSNYRAYLIDHLNDIIDSLGPDRVDGLFLDIFSLVPCDCPRCREQMERHGFDHTDIRQRERYSAILLDEFRREVHDLVDDRVPGATLFFNGGHIGPSNKTSLDTFTHLEVESLPSGFWGYDNFPIVMRYVRNLGKPVVGMTGKFHTAWGDFHSLKNEDAMEYECFQMLTMGAGCSIGDQLHPRGGLSDATYDLIGRVYSQVEALEPYTLDTDTMADIAVMTPEREWNMDSALSDSLIGANRMLTELGCQFDIIDPDMDFTRYGLIVLPDEIVSSPELQRKLLDYVHGGGKVVGTYMSMDNGCDETNPLYGNRMLGDSYWDRDFIMPNDEVGARLPKEEFVMYERGARVRTAGSRVLLDSVEPYFNREGRYFCSHLHAPSTGRVGFPAATRYGDVVYFSHPLFRIYKDFAPSWVKAIFADVLDLLMPRQLVRKEDGHTVSGLEVQLRRSGSRNSLMLHCLYYPCKKSAANLYTIDEKVPLFDQRVRVYVGDAEIESVRAIRQGEVISERDYTVADGYVDLNIPKIDGYEIIELSLK

SEQ ID NO: 64 - protein, 708 aa, unidentified
MSDVQNAAQQAAIERATAEAGASTDFSQFAGSSLPFGNGQVLLDQSTGAPIAFVNEDNPNRHYLLDDSVSWHTSDQYWGSGYVVTTSGGSQWNTPQVYGITNGLEQQTFALCGAGLALDVTRSSGERLTERYEWRNVSNQPVTITGLGIQTPLNDRYPGARKALRECVHAHIFAGGDWSWVLAEPMDGRGARLGLIVTEGAINAYAIQSRNQVTYSDIRGHIVLQVTDYALNPDAFGGQPTITLEPGASYALTWELGWYADEDAFLAATIAPASFSSLSATVGGHIDIATNRAVTALDDGLTVERTVSGVRLTAEQAGTYQLQLVDGDHRAHTEVLFHKTLRDTVNDRARYILEHQVAYERVGALAGAIVATDTRTGQHVLDPSWDDWSDGSERIAMPVLLQRALNLGLLDESLREPAQRAADAWRTFAESALIDASGAVRRGSGLPESKFGGRIYDCPWIISFFTEHYLATKDAHDLDMAVNIADRAAEIGGEHFLSIEFAESSAALARVLKQTGRTEETQRLLDRVVVSADYFLGLGIDLPYHEVSYEQSITAPLVSLLLEAHRITGRAEYLDGAVERLDWLLAFSGPQPDSRLYGVAIRHWDGYWFGINRQFGDVFPHYWSTLTAEVLARLPQERRSARTDDLAKAIFEANMANYGADGSATCAYVFPSNVDGRSMHQADPLANDQDWHLNIWMRMIAEEGFSAL